(19) United States
(12) Patent Application Publication
(10) Pub. No.: US 2025/0366946 A1
(43) Pub. Date: Dec. 4, 2025

(54) REUSABLE COVER FOR SURGICAL NAVIGATION DEVICE

(71) Applicant: OrthAlign, Inc., Irvine, CA (US)

(72) Inventors: Brian Olivo, Redlands, CA (US); Robert Towle, Laguna Beach, CA (US); James Gannoe, Laguna Niguel, CA (US)

(21) Appl. No.: 19/221,251

(22) Filed: May 28, 2025

Related U.S. Application Data
(60) Provisional application No. 63/652,920, filed on May 29, 2024.

(51) Int. Cl. A61B 50/30; A61B 50/00; A61B 90/00; H04L 9/40

(52) U.S. Cl. CPC A61B 50/30; H04L 63/0428; A61B 2050/006; A61B 2090/0813; A61B 2560/0214

(57) ABSTRACT

In some embodiments, a system for a surgical procedure is provided. The system can include a sterile cover comprising a housing and a lid. The system can include a surgical navigation device comprising at least one inertial sensor and a display. In some embodiments, the surgical navigation device is configured to be inserted into the housing of the sterile cover. In some embodiments, the lid is configured to close to provide a sterile barrier separating the surgical navigation device from the sterile field. In some embodiments, positional and rotation fixation is applied to the surgical navigation device for accuracy of the at least one inertial sensor. In some embodiments, a method for a surgical procedure is provided.

### INCORPORATION BY REFERENCE TO ANY PRIORITY APPLICATIONS

This application claims the benefit under 35 U.S.C. § 119 (e) to U.S. Provisional Application No. 63/652,920, filed May 29, 2024, which is hereby incorporated by reference in its entirety. Any and all applications for which a foreign or domestic priority claim is identified in the Application Data Sheet as filed with the present application is hereby incorporated by reference in its entirety under 37 CFR 1.57.

### BACKGROUND

### Field

The present application is directed to systems and methods for joint replacement, e.g., to systems and methods for of joints of the extremities including knee joint replacement and hip joint replacement which utilize a surgical navigation device or devices.

### Description of the Related Art

Joint replacement procedures, including but not limited to knee joint replacement and hip replacement procedures, are commonly used to replace a patient's joint with a prosthetic joint component or components. Such procedures often use a system or systems of surgical tools and devices, including but not limited to cutting guides to make surgical cuts along a portion or portions of the patient's bone for knee joint replacement and impactors to place prosthetic joint components relative to the patient's anatomy for hip joint replacement.

Current systems and methods often use expensive, complex, bulky, and/or massive computer navigation systems which require a computer or computers, as well as three-dimensional imaging, to track a spatial location and/or movement of a surgical instrument or landmark in the human body. These systems are used generally to assist a user to determine where in space a tool or landmark is located, and often require extensive training, cost, and room.

Where such complex and costly system are not used, simple methods are used, such “eyeballing” the alignment of rods with anatomical features. These simple methods are not sufficiently accurate to reliably align and place implant components and the bones to which such components are attached.

### SUMMARY

Accordingly, there is a lack of devices, systems and methods that can be used to accurately position components of prosthetic joints without overly complicating the procedures, crowding the medical personnel, and/or burdening the physician of health-care facility with the great cost of complex navigation systems.

In some embodiments, a system for a surgical procedure is provided. The system can include a sterile cover comprising a housing and a lid. The system can include a surgical navigation device comprising at least one inertial sensor and a display. In some embodiments, the surgical navigation device is configured to be inserted into the housing of the sterile cover. In some embodiments, the lid is configured to close to provide a sterile barrier separating the surgical navigation device from a sterile field.

In some embodiments, the system can include a transfer funnel. In some embodiments, the transfer funnel is configured to prevent contact between the surgical navigation device and the sterile cover. In some embodiments, the transfer funnel comprises an alignment slot configured to prevent inserting the surgical navigation device in an incorrect orientation relative to the sterile cover. In some embodiments, the transfer funnel comprises instructions to the user related to the orientation of the surgical navigation device. In some embodiments, the transfer funnel comprises instructions to the user related to coupling a battery pack to the surgical navigation device. In some embodiments, the housing comprises one or more magnets for coupling to a bracket of a jig. In some embodiments, the housing comprises a seal between the lid and the housing of the sterile cover. In some embodiments, the housing comprises a front housing, a rear housing, and a seal between the front housing and the rear housing. In some embodiments, the housing comprises a transparent, touch compatible layer. In some embodiments, the system can include a battery pack. In some embodiments, the battery pack comprises a counter, wherein the battery pack is programmed for a single use. In some embodiments, the battery pack provides licensing. In some embodiments, the battery pack provides authentication. In some embodiments, the lid is configured to pivot approximately 90 degrees relative to the housing. In some embodiments, the lid is configured to pivot approximately 180 degrees relative to the housing. In some embodiments, the housing comprises projections along two sides of the housing configured to engage grooves along two sides of the lid. In some embodiments, the housing comprises projections along three sides of the housing configured to engage grooves along three sides of the lid. In some embodiments, the housing and the lid form a hinge along a front side of sterile cover. In some embodiments, the housing and the lid form a hinge along a back side of sterile cover.

In some embodiments, a system for a surgical procedure is provided. The system can include a sterile cover comprising a housing and a lid. The system can include a surgical navigation device comprising at least one inertial sensor and a display. In some embodiments, the surgical navigation device is configured to be inserted into the housing of the sterile cover. In some embodiments, the lid is configured to close. In some embodiments, positional and rotation fixation is applied to the surgical navigation device for accuracy of the at least one inertial sensor.

In some embodiments, the surgical navigation device is configured to rest against one or more angled ramps configured to drive the surgical navigation device forward. In some embodiments, the lid comprise one or more alignment projections configured to rest against the surgical navigation device to drive the surgical navigation device down and forward. In some embodiments, the housing comprises one or more alignment features. In some embodiments, the housing comprises one or more angled ramps. In some embodiments, the housing comprises a transparent layer that overlies the display. In some embodiments, the sterile cover comprises one or more features that drive the device toward the transparent layer.

In some embodiments, a method for a surgical procedure is provided. The method can include providing a sterile cover comprising a housing and a lid. The method can include providing a surgical navigation device comprising at least one inertial sensor and a display. The method can include inserting the surgical navigation device into the housing of the sterile cover. The method can include closing the lid.

In some embodiments, the method can include assembling a transfer funnel onto the sterile cover before inserting the surgical navigation device into the housing of the sterile cover. In some embodiments, the method can include removing the transfer funnel from the sterile cover after inserting the surgical navigation device into the housing of the sterile cover. In some embodiments, the transfer funnel is sterile. In some embodiments, the method can include attaching a bracket to the sterile cover. In some embodiments, the method can include performing the surgical procedure. In some embodiments, the method can include disposing the sterile cover after the surgical procedure and cleaning the surgical navigation device for reuse after the surgical procedure. In some embodiments, inserting the surgical navigation device into the housing of the sterile cover comprises inserting the surgical navigation device in a single orientation. In some embodiments, the method can include coupling a battery pack to the surgical navigation device. In some embodiments, the battery pack comprises data that provides the type of license. In some embodiments, the battery pack and the surgical navigation device compute a keyed-hash message authentication code to verify the authenticity of the battery pack.

In some embodiments, a system for a surgical procedure is provided. The system can include a surgical navigation device comprising at least one inertial sensor and a display. The system can include a battery pack. In some embodiments, the battery pack is configured to be coupled to the surgical navigation device.

In some embodiments, the battery pack comprises data that provides the type of license. In some embodiments, the battery pack and the surgical navigation device compute a keyed-hash message authentication code to verify the authenticity of the battery pack. In some embodiments, the battery pack comprises a counter, wherein the battery pack is programmed for a single use. In some embodiments, the battery pack provides licensing. In some embodiments, the battery pack provides authentication. In some embodiments, the battery pack is configured to fit within an attachment groove of the surgical navigation device. In some embodiments, the battery pack is configured to supply power to the display of the surgical navigation device. In some embodiments, the battery pack is configured to communicate with a co-processor integrated circuit of the surgical navigation device. In some embodiments, the battery pack comprises a Secure Hash Algorithm engine. In some embodiments, the battery pack comprises electrically erasable programmable read-only memory (EEPROM).

In some embodiments, a single use kit is provided. The single use kit can include a sterile cover comprising a housing and a lid. The single use kit can include a transfer funnel. The single use kit can include a battery pack configured to provide power to a surgical navigation device.

In some embodiments, the sterile cover and the transfer funnel are pre-assembled. In some embodiments, the sterile cover and the transfer funnel are in a sterile packaging within the single use kit. In some embodiments, the battery pack is coupled to a carton insert within the single use kit. In some embodiments, the battery pack is non-sterile within the single use kit. In some embodiments, the transfer funnel is sterile within the single use kit.

### BRIEF DESCRIPTION OF THE DRAWINGS

FIG. 1 is a front view of a system.

FIG. 2 is a back view of the system of FIG. 1.

FIG. 3 is a front perspective view of a transfer funnel.

FIG. 4 is a back perspective view of the transfer funnel of FIG. 3.

FIG. 5 is another perspective view of the transfer funnel of FIG. 3.

FIG. 6 is a front perspective view of a sterile cover.

FIG. 7 is a back perspective view of the sterile cover of FIG. 6.

FIG. 8 is an exploded perspective view of the sterile cover of FIG. 6.

FIG. 9 is a perspective view of a lid of the sterile cover of FIG. 6.

FIG. 10 is a perspective view of a housing of the sterile cover of FIG. 6.

FIG. 11 is a front perspective view of a surgical navigation device.

FIG. 12 is a back view of the surgical navigation device of FIG. 11.

FIG. 13 is another back view of the surgical navigation device of FIG. 11.

FIG. 14 is a front perspective view of a battery pack.

FIG. 15 is a back view of the battery pack of FIG. 14.

FIG. 16 is a back perspective view of the battery pack of FIG. 14.

FIG. 17 is a cross sectional view of the sterile cover of FIG. 6 and the surgical navigation device of FIG. 11.

FIG. 18 is another cross-sectional view of the sterile cover of FIG. 6 and the surgical navigation device of FIG. 11.

FIG. 19 is a top view of the sterile cover of FIG. 6 and the surgical navigation device of FIG. 11.

FIG. 20 is a cross-sectional view of the transfer funnel of FIG. 3 and the sterile cover of FIG. 6.

FIG. 21 is another cross-sectional view of the transfer funnel of FIG. 3 and the sterile cover of FIG. 6.

FIG. 22 is a method of use.

FIG. 23 is a method of use of the transfer funnel of FIG. 3 and the sterile cover of FIG. 6.

FIG. 24 is a method of use of the transfer funnel of FIG. 3 and the sterile cover of FIG. 6.

FIG. 25 is a method of use of the transfer funnel of FIG. 3, the sterile cover of FIG. 6, and the surgical navigation device of FIG. 11.

FIG. 26 is a method of use of the transfer funnel of FIG. 3, the sterile cover of FIG. 6, and the surgical navigation device of FIG. 11.

FIG. 27 is a method of use of the transfer funnel of FIG. 3, the sterile cover of FIG. 6, and the surgical navigation device of FIG. 11.

FIG. 28 is a method of use of the sterile cover of FIG. 6 and the surgical navigation device of FIG. 11.

FIG. 29 is a method of use of the sterile cover of FIG. 6 and the surgical navigation device of FIG. 11.

FIG. 30 is a method of use of the sterile cover of FIG. 6, the surgical navigation device of FIG. 11, and a bracket.

FIG. 31 is a method of use of the sterile cover of FIG. 6, the surgical navigation device of FIG. 11, and the bracket of FIG. 30.

FIG. 32 is a front view of a system.

FIG. 33 is a back view of the system of FIG. 32.

FIG. 34 is a front perspective view of a transfer funnel.

FIG. 35 is a back perspective view of the transfer funnel of FIG. 34.

FIG. 36 is another perspective view of the transfer funnel of FIG. 34.

FIG. 37 is a front perspective view of a sterile cover.

FIG. 38 is a back perspective view of the sterile cover of FIG. 37.

FIG. 39 is an exploded perspective view of the sterile cover of FIG. 37.

FIG. 40A is a top perspective view of a lid of the sterile cover of FIG. 37.

FIG. 40B is a bottom perspective view of the lid of the sterile cover of FIG. 37.

FIG. 41 is a perspective view of a housing of the sterile cover of FIG. 37.

FIG. 42 is a front perspective view of a surgical navigation device.

FIG. 43 is a back view of the surgical navigation device of FIG. 42.

FIG. 44 is another back view of the surgical navigation device of FIG. 42.

FIG. 45 is a front perspective view of a battery pack.

FIG. 46 is a back view of the battery pack of FIG. 45.

FIG. 47 is a back perspective view of the battery pack of FIG. 45.

FIG. 48 is a cross sectional view of the sterile cover of FIG. 37 and the surgical navigation device of FIG. 42.

FIG. 49 is another cross-sectional view of the sterile cover of FIG. 37 and the surgical navigation device of FIG. 42.

FIG. 50 is a top view of the sterile cover of FIG. 37 and the surgical navigation device of FIG. 42.

FIG. 51 is a cross-sectional view of the transfer funnel of FIG. 34 and the sterile cover of FIG. 37.

FIG. 52 is another cross-sectional view of the transfer funnel of FIG. 34 and the sterile cover of FIG. 37.

FIG. 53 is a method of use.

FIG. 54 is a method of use of the transfer funnel of FIG. 34 and the sterile cover of FIG. 37.

FIG. 55 is a method of use of the transfer funnel of FIG. 34 and the sterile cover of FIG. 37.

FIG. 56 is a method of use of the transfer funnel of FIG. 34, the sterile cover of FIG. 37, and the surgical navigation device of FIG. 42.

FIG. 57 is a method of use of the transfer funnel of FIG. 34, the sterile cover of FIG. 37, and the surgical navigation device of FIG. 42.

FIG. 58 is a method of use of the transfer funnel of FIG. 34, the sterile cover of FIG. 37, and the surgical navigation device of FIG. 42.

FIG. 59 is a method of use of the sterile cover of FIG. 37 and the surgical navigation device of FIG. 42.

FIG. 60 is a method of use of the sterile cover of FIG. 37 and the surgical navigation device of FIG. 42.

FIG. 61 is a method of use of the sterile cover of FIG. 37, the surgical navigation device of FIG. 42, and a bracket.

FIG. 62 is a method of use of the sterile cover of FIG. 37, the surgical navigation device of FIG. 42, and the bracket of FIG. 61.

FIGS. 63A and 63B are views of a single use kit.

FIGS. 64A and 64B are views of the surgical navigation device of FIG. 42.

FIG. 65 is a view of a storage tray.

FIG. 66 is a view of a sterile packaging with the transfer funnel of FIG. 34 and the sterile cover of FIG. 37 and a non-sterile packaging with the battery pack of FIG. 45.

FIG. 67 is a view of the transfer funnel of FIG. 34, the sterile cover of FIG. 37, and the battery pack of FIG. 45.

FIG. 68 are views of the surgical navigation device of FIG. 42.

FIG. 69 is a method of use.

### DETAILED DESCRIPTION

The following sections describe in detail systems and methods for joint replacement procedures including but not limited to knee joint replacement and hip replacement procedures. The joint often requires replacement in the form of prosthetic components due to strain, stress, wear, deformation, misalignment, and/or other conditions in the joint. Prosthetic knee joint components are designed to replace a distal portion or portions of a femur and/or a proximal portion or portions of a tibia in knee replacement procedures. Prosthetic knee joint components are designed to replace an acetabular socket and/or a proximal portion or portions of a femur in hip replacement procedures.

Prior to replacing the knee joint with prosthetic components, surgical cuts commonly called resections are generally made with a cutting tool or tools along a portion or portions of both the proximal tibia and distal femur. These cuts are made to prepare the tibia and femur for the prosthetic components relative to the patient's anatomy. The desired orientation and/or position of these cuts, and of the prosthetic components, can be determined pre-operatively and based, for example, on a mechanical axis running through an individual patient's leg for knee replacement. Once the desired locations of these cuts are determined pre-operatively, the surgeon can use the systems and methods described herein to make these cuts accurately. After these cuts are made, the prosthetic components can be attached and/or secured to the tibia and femur.

Prior to replacing the hip joint with prosthetic components, the acetabular socket can be prepared. After the socket is prepared, the prosthetic components such as an acetabular cup can be oriented relative to the patient's anatomy. The desired orientation and/or position of the prosthetic components can be determined pre-operatively and based, for example, on a patient-specific safe zone. Once the desired angles are determined pre-operatively, the surgeon can use the systems and methods described herein to position the prosthetic components.

While the systems and methods are described in the context of a knee joint replacement and hip replacement procedures, the systems and/or their components and methods can similarly be used in other types of surgical procedures. One or more components can be used together in systems and methods described herein. While examples of components are described below, the systems and methods can be used with additional or alternative or fewer components.

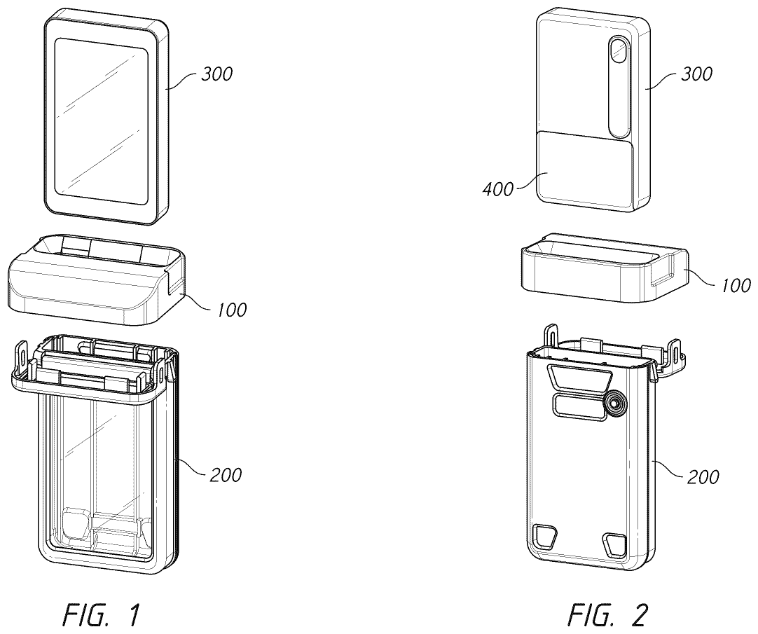

FIG. 1 is a view of a system. FIG. 2 is another view of the system. The system can include a transfer funnel 100. The system can include a sterile cover 200. The system can include a surgical navigation device 300. The system 100 can include a battery pack 400. The sterile cover 200 can be designed to cover the surgical navigation device 300 during a surgical procedure. The surgical navigation device 300 can include a touchscreen and other electronics such as inertial sensors which make sterilization unfeasible. The sterile cover 200 can be a single use sterile barrier separating non-sterile surgical navigation device 300 from the sterile field. The system can include positional and rotational features that align the surgical navigation device 300 within the sterile cover 200 for sensor accuracy. These features can include features of the sterile cover 200 that push the surgical navigation device 300 downward and forward. The sterile cover 200 can have one or more liquid resistant seals. The sterile cover 200 can have a transparent, touch compatible screen window for operation of the surgical navigation device 300 therethrough. The sterile cover 200 can be sterile and single use. The surgical navigation device 300 can be reusable. The surgical navigation device 300 can include inertial sensors that determine orientation of the surgical navigation device. The surgical navigation device 300 can be used in a joint replacement procedure, such as a knee replacement procedure to position a cutting block or a hip replacement procedure to position an acetabular cup. The transfer funnel 100 can limit or prevent accidental contact between external surfaces of the sterile cover 200 and the surgical navigation device 300 during aseptic transfer. The transfer funnel 100 can provide alignment features to limit or prevent inserting surgical navigation device in incorrect orientation relative to the sterile cover 200. The transfer funnel 100 can be sterile and single use.

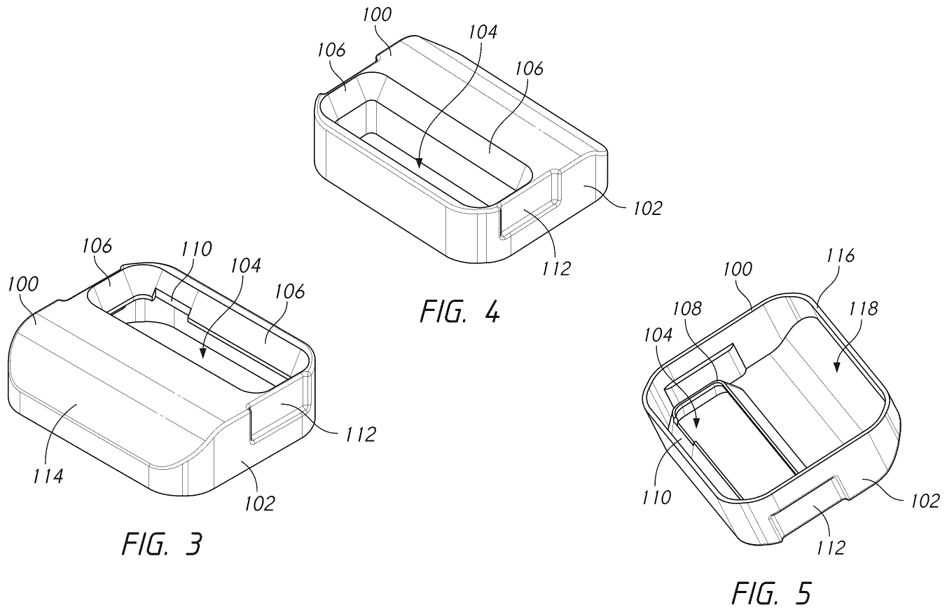

FIG. 3 is front perspective view of a transfer funnel 100. FIG. 4 is back perspective view of the transfer funnel 100. FIG. 5 is another perspective view of the transfer funnel 100. The transfer funnel 100 can include a body 102. The body 102 can be unitarily or monolithically formed. The transfer funnel 100 can include a lumen 104. The lumen 104 can extend entirely through the body 102. The lumen 104 can be elongated. The lumen 104 can be generally rectangular. The lumen 104 can include rounded edges. The transfer funnel 100 can include tapered edges 106 extending toward the lumen 104. The tapered edges 106 can guide components through the lumen 104, as described herein. The tapered edges 106 can function as a funnel. The tapered edges 106 can include an inclined surface with a constant angle. The tapered edges 106 can include a concave surface. The tapered edges 106 can include a convex surface.

The transfer funnel 100 can include a ledge 108. The ledge 108 can be formed between the lumen 104 and the tapered edges 106. The tapered edges 106 can extend inward from the lumen 104. The ledge 108 can function as a stop for other components, as described herein. The ledge 108 can rest against other components. The ledge 108 can extend around the entire perimeter of the lumen 104. The ledge 108 can extend around a portion of the perimeter of the lumen 104. The ledge 108 can form a flat surface for abutting other components, as described herein.

The transfer funnel 100 can include an alignment slot 110. The alignment slot 110 can interrupt the tapered edge 106. The alignment slot 110 can ensure that components inserted into the transfer funnel 100 are correctly oriented, as described herein. The alignment slot 110 can allow the user to view other components, as described herein. The alignment slot 110 can interrupt the ledge 108.

The transfer funnel 100 can include one or more finger grips 112. The finger grips 112 can allow the user to raise and lower the transfer funnel 100 relative to other components, as described herein. The finger grips 112 can be located near the lumen 104. The finger grips 112 can be located on an outer surface of the transfer funnel 100. The finger grips 112 can be unitarily or monolithically formed with the body 102.

The transfer funnel 100 can include an extension 114. The extension 114 can function as a cover for other components, as described herein. The extension 114 can receive other components, as described herein. The extension 114 can include a curved surface. The extension 114 can include curved edges. The lumen 104 can be offset relative to a midline of the body 102. The lumen 104 can be positioned on one side of the midline of the body 102 and the extension 114 can be positioned on the other side of the midline of the body 102.

The transfer funnel 100 can include a lower edge 116. In some embodiments, the lower edge 116 can function as a shield for other components. In some embodiments, the lower edge 116 can extend below other components. The lower edge 116 can extend around the entire perimeter of the body 102. The lower edge 116 can extend around a portion of the perimeter of the body 102. In some embodiments, the lower edge 116 can form a flat surface for covering other components. The transfer funnel 100 can include a cavity 118. The cavity 118 can accommodate other components, as described herein. In some embodiments, the cavity 118 can be partially enclosed when the ledge 108 contacts other components.

The transfer funnel 100 can prevent accidental contact between external surfaces of the sterile cover 200 and the non-sterile surgical navigation device 300 during aseptic transfer. The transfer funnel 100 can cover at least a portion of the sterile cover 200 when the sterile cover 200 is opened, as described herein. The transfer funnel 100 can cover the entire sterile cover 200 from a top perspective. The transfer funnel 100 can cover the entire sterile cover 200 when viewed from the direction of insertion of the surgical navigation device 300. The transfer funnel 100 can provide alignment features to prevent inserting the surgical navigation device 300 in an incorrect orientation relative to the sterile cover 200. The transfer funnel 100 can be sterile. The transfer funnel 100 can be single use.

The transfer funnel 100 can be a separate component from other components of the system. The transfer funnel 100 can be a separate, sterile shield. The transfer funnel 100 can be disposable. The transfer funnel 100 can be manufactured and packaged in a sterile state.

The transfer funnel 100 can provide a temporary barrier between the sterile cover 200 and the surgical navigation device 300 during insertion. The transfer funnel 100 can limit or prevent accidental contact between the surgical navigation device 300 and outside surfaces of the sterile cover 200. Once the surgical navigation device 300 is inserted, the transfer funnel 100 can be removed and discarded allowing the sterile cover 200 to be closed.

In some embodiments, the transfer funnel 100 can be disposable. The transfer funnel 100 can be single use. The transfer funnel 100 can be manufactured and packaged in a sterile state. Once the transfer funnel 100 has been used, the transfer funnel 100 can be discarded. The transfer funnel 100 can be a single use sterile barrier separating non-sterile surgical navigation device 300 from the sterile cover 200. The transfer funnel 100 can be intended for use with one single patient. The transfer funnel 100 can be intended for use with one single procedure. The transfer funnel 100 can be provided to the user in sterile medical device packaging. The transfer funnel 100 can be provided to the user with instructions for single use.

In some embodiments, the transfer funnel 100 can be reusable. The transfer funnel 100 can be configured for use with multiple patients. The transfer funnel 100 can be configured for use with multiple procedures. The transfer funnel 100 can be sterilized after use. The transfer funnel 100 can be sterilized using a variety of methods including thermal sterilization such as moist heat including high-pressure steam or dry heat, radiation, liquid chemical solutions including glutaraldehydes or formaldehydes, gas such ethylene oxide gas (ETO), vaporized hydrogen peroxide, ozone, UV chambers, chlorine dioxide gas, vaporized peracetic acid, and nitrogen dioxide gas, and combinations of methods thereof. The transfer funnel 100 can be autoclaved. The transfer funnel 100 can be subjected to high temperatures and pressure in order to sterilize the transfer funnel 100 for reuse. The transfer funnel 100 can be subjected to any sterilization method. The transfer funnel 100 can be sterilized on site, at the hospital where the surgical procedure takes place. The transfer funnel 100 can be sterilized off-site. The transfer funnel 100 can comprise materials such as polymers. The transfer funnel 100 can have multiple layers. The transfer funnel 100 can be sterilized using methods that effectively sterilizes and does not damage the transfer funnel 100. The transfer funnel 100 can be provided to the user in sterile medical device packaging after sterilization. The transfer funnel 100 can be provided to the user with instructions for multiple uses. The transfer funnel 100 can be provided to the user with instructions for sterilization.

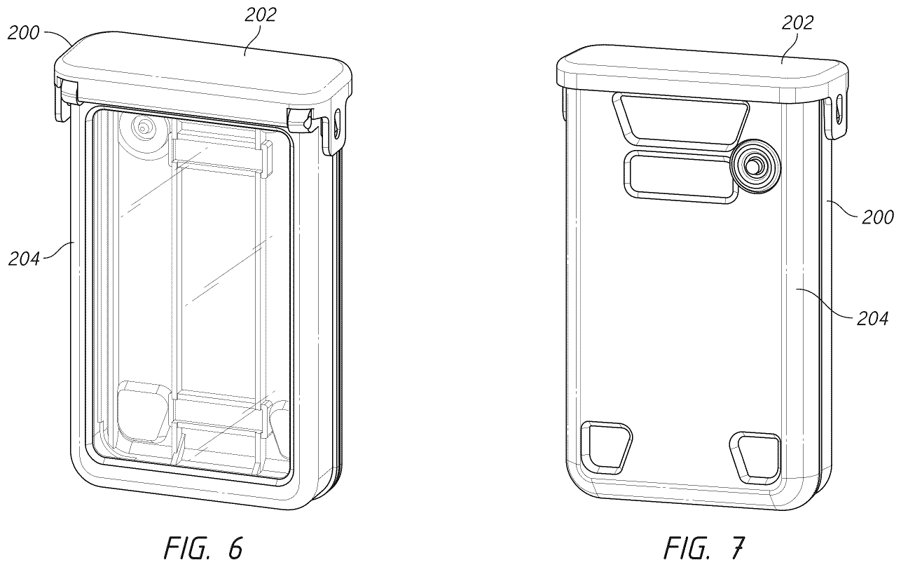

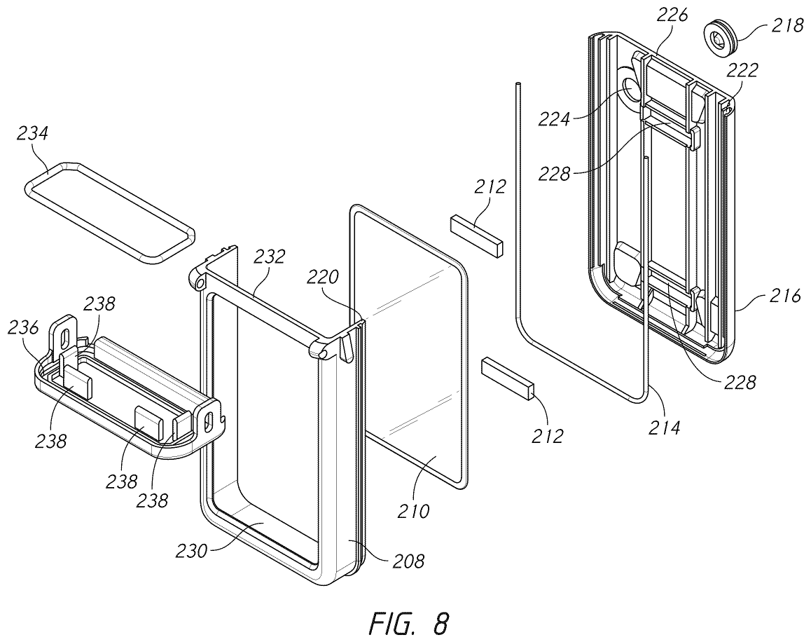

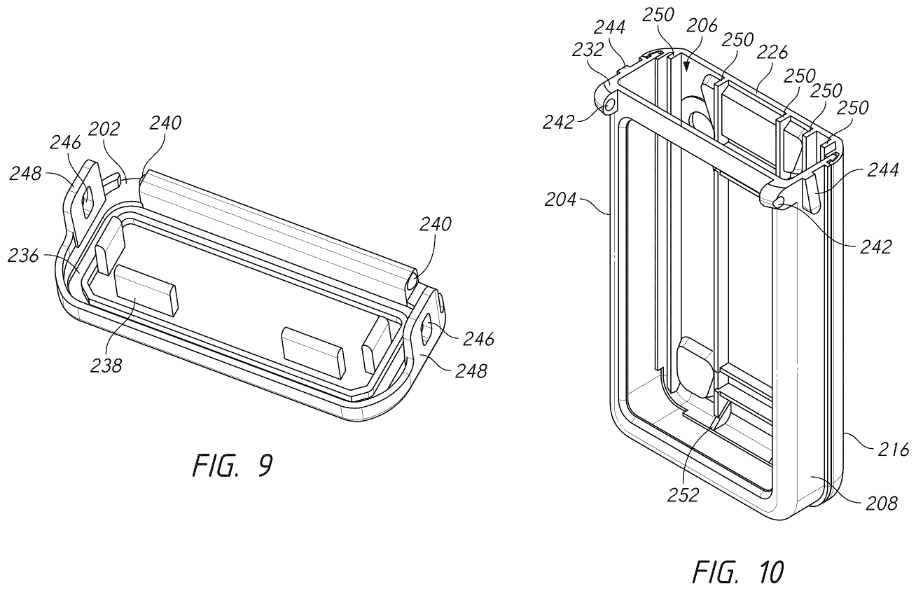

FIG. 6 is front perspective view of the sterile cover 200. FIG. 7 is back perspective view of the sterile cover 200. FIG. 8 is an exploded perspective view of the sterile cover 200. FIG. 9 is perspective view of a lid 202 of the sterile cover 200. FIG. 10 is perspective view of a housing 204 of the sterile cover 200.

The sterile cover 200 can be configured to open and close. The sterile cover 200 can include the lid 202. The sterile cover 200 can include the housing 204. The lid 202 can be coupled to the housing 204. The lid 202 can be pivotally coupled to the housing 204. The lid 202 can be coupled to an upper portion of the housing 204. The lid 202 can be coupled to a shorter side of the housing 204. The lid 202 can be coupled transverse to a longitudinal axis of the housing 204. The lid 202 can pivot relative to the housing 204. The lid 202 can swing open or close. The lid 202 can be opened to allow insertion and removal of components, described herein.

The housing 204 can including an opening 206, as shown in FIG. 10. The opening 206 can allow access into the housing 204. The opening 206 is accessible when the lid 202 is opened. The lid 202 can cover the opening 206 in the housing 204 when the lid is closed 202. The opening 206 can be elongated. The opening 206 can be generally rectangular. The opening 206 can include rounded edges. The lid 202 can seal the housing 204. The housing 204 can be partially enclosed when the lid 202 is opened. The sterile cover 200 can be fully enclosed when the lid 202 is closed. The housing 202 and the lid 204 can form a fully enclosed structure when the lid 202 is closed.

The housing 204 can include several components. The housing 204 can include a front housing 208. The housing 204 can include a transparent layer 210. The housing 204 can include one or more magnets 212. The housing 204 can include a housing seal gasket 214. The housing 204 can include a rear housing 216. The housing 204 can include a soft touch button 218.

The front housing 208 and the rear housing 216 can be coupled. The front housing 208 and the rear housing 216 can form a snap fit. The front housing 208 can include an interlocking projection 220. The rear housing 216 can include an interlocking groove 222. The interlocking projection 220 and the interlocking groove 222 can be flexible parts that can be pushed together to interlock. In some embodiments, the interlocking projection 220 and the interlocking groove 222 are configured to be permanent. The interlocking projection 220 and the interlocking groove 222 are configured to resist disassembly during use.

The housing seal gasket 214 can be disposed between the front housing 208 and the rear housing 216. The housing seal gasket 214 can form a liquid resistant seal between the front housing 208 and the rear housing 216. The housing seal gasket 214 can be disposed within the interlocking groove 222 of the rear housing 216. The front housing 208 can include the interlocking projection 220 that compresses the housing seal gasket 214. The housing seal gasket 214 can be compressed when the interlocking projection 220 and the interlocking groove 222 interlock.

The rear housing 216 can include a lumen 224. The lumen 224 can receive the soft touch button 218. The soft touch button 218 can form a liquid resistant seal between the soft touch button 218 and the rear housing 216. The soft touch button 218 can allow the user to depress a button of the surgical navigation device 300 when the surgical navigation device 300 is disposed within the sterile cover 200. The soft touch button 218 can allow the user to power on or power off the surgical navigation device 300 when the surgical navigation device 300 is disposed within the sterile cover 200. The rear housing 216 can include a top surface 226. The top surface 226 can be flat. The top surface 226 can abut other components, as described herein.

The rear housing 216 can include one or more attachment grooves 228. The attachment groove 228 can be configured to receive the magnet 212. The rear housing 216 can include two attachment grooves 228. The housing 204 can include two magnets 212. The magnets 212 can be spaced apart. The magnets 212 can be parallel. The attachment magnets 212 can be aligned. The magnets 212 can couple to a bracket of a jig, as described herein. The front housing 208 can include a lumen 230. The lumen 230 of the front housing 208 can allow the user to see through the front housing 208. The transparent layer 210 can be positioned relative to the lumen 230. The lumen 230 of the front housing 208 can allow the user to see through the transparent layer 210. The transparent layer 210 can be coupled to the front housing 208. The transparent layer 210 can form a liquid resistant seal between the transparent layer 210 and the front housing 208. The transparent layer 210 can be adhered to the front housing 208, such as the use of an adhesive. The front housing 208 can include a top surface 232. The top surface 232 can be flat.

The transparent layer 210 can be touch compatible. The transparent layer 210 can be a thin plastic layer. The transparent layer 210 can cover a portion of the surgical navigation device 300 when the surgical navigation device 300 is disposed within the sterile cover 200. The transparent layer 210 can cover a touch screen display of the surgical navigation device 300 when the surgical navigation device 300 is disposed within the sterile cover 200. The transparent layer 210 can allow the user to manipulate touch screen elements by pressing against transparent layer 210 of the housing 204. The transparent layer 210 can include a touch-sensitive overlay which covers the surgical navigation device 300 to enable the surgical navigation device 300 to be operated as a touch screen when the surgical navigation device 300 is disposed within the sterile cover 200. The sterile cover 200 can include the touch compatible, transparent layer 210.

The lid 202 can include several components. The lid 202 can include a lid seal gasket 234. The lid seal gasket 234 can be disposed between the housing 204 and the lid 202. The lid seal gasket 234 can form a liquid resistant seal between the housing 204 and the lid 202. The lid seal gasket 234 can be disposed within a groove 236 of the lid 202. The top surface 232 of the front housing 208 and the top surface 226 of the rear housing 216 can compresses the lid seal gasket 234 when the lid 202 is closed. The lid seal gasket 234 can be compressed between the lid 202 and the housing 204 when the lid 202 is close. The sterile cover 200 can include a liquid resistant seal on the lid 202 and the housing 204.

The lid 202 can include one or more alignment projections 238 (e.g., one projection, two projections, three projections, four projections, five projections, six projections, seven projections, eight projections, ten projections, twelve projections, or any range of the foregoing values). The lid 202 can include one or more alignment projections 238 along one or more edges (e.g., one edge, two edges, three edges, four edges, or any range of two of the foregoing values). The lid 202 can include one or more alignment projections 238 along three edges. The lid 202 can include one or more alignment projections 238 along a first side edge. The lid 202 can include one or more alignment projections 238 along a second side edge, opposite the first side edge. The lid 202 can include one or more alignment projections 238 along an edge opposite a pivot axis. The alignment projections 238 can facilitate the proper orientation of components, as described herein. The alignment projections 238 can facilitate alignment of the surgical navigation device 300 within the sterile cover 200, as described herein.

The lid 202 and the housing 204 can form a hinge. The lid 202 and the housing 204 can form a mechanical bearing. The lid 202 and the housing 204 can allow for pivotal movement. The lid 202 and the housing 204 can allow for a limited range of rotation. The lid 202 can rotate about the housing 204 approximately 180 degrees. The lid 202 can rotate about the housing 204 to open to form a flat surface. The lid 202 can rotate about the housing 204 to side by side with the opening 206 of the housing 204. The lid 202 can rotate about the housing 204 to side by side with the top surfaces 223, 232 of the housing 204. The lid 202 can rotate about the housing 204 about a fixed axis of rotation. The lid 202 can rotate about one axis, while all other translations and rotations are prevented. The lid 202 and the housing 204 can have one degree of freedom.

The lid 202 can include two pins 240, as shown in FIG. 9. The pins 240 can be aligned along the axis of rotation. The front housing 208 can include two sockets 242. The sockets 242 can be aligned along the axis of rotation. The sockets 242 can receive the pins 240 to form a hinge joint. In other embodiments, the lid 202 can include sockets and the front housing 208 can include pins. In other embodiments, the lid 202 and the housing 204 are joined via an axle. The lid 202 and the housing 204 can include barrels to receive the axle. Other pivot joints are contemplated.

The lid 202 and the housing 204 can form a latch. The lid 202 and the housing 204 can form a catch. The lid 202 and the housing 204 can form a locking mechanism. The lid 202 and the housing 204 can from a mechanical fastener that allows for reversable separation of the lid 202 and the housing 204. The lid 202 and the front housing 208 can form a cantilever snap fit.

The front housing 208 can include one or more projections 244 (e.g., one projection, two projections, three projections, four projections, or any range of two of the foregoing values). The front housing 208 can include two projections 244. The two projections 244 can be on opposite sides of the front housing 208. The two projections 244 can be diametrically opposed. The two projections 244 can be aligned along an axis. The two projections 244 can have two axes of symmetry.

The lid 202 can include one or more grooves 246 (e.g., one groove, two grooves, three grooves, four grooves, or any range of two of the foregoing values). The lid 202 can include one or more tabs 248 (e.g., one tab, two tabs, three tabs, four tabs, or any range of two of the foregoing values). The number of projections 244 can correspond to the number of grooves 246. The number of projections 244 can correspond to the number of tabs 248. The tab 248 can include the groove 246. The lid 202 can include two grooves 246. The lid 202 can include two tabs 248. The two tabs 248 can be on opposite sides of the lid 202. The two tabs 248 can be diametrically opposed. The two tabs 248 can be aligned along an axis. The two tabs 248 can have two axes of symmetry.

The projection 244 and the groove 246 of the tab 248 can be flexible parts that can be pushed together to interlock. The projection 244 and the groove 246 of the tab 248 can be flexible parts that can be pulled apart to release. In some embodiments, the projection 244 and the groove 246 of the tab 248 are configured to be detachable. The projection 244 and the groove 246 of the tab 248 are configured to be reversable upon the user applying a force to the tab 248. The projection 244 and the groove 246 of the tab 248 are configured to be reversable upon the user applying a force to disengage the groove 246 of the tab 248 from the projection 244.

The housing 204 can form a flat surface. The rear housing 216 can include the top surface 226. The top surface 226 can be flat. The front housing 208 can include a top surface 232. The top surface 232 can be flat. The top surfaces 226, 232 of the housing 204 can be aligned to form a flat surface. The top surfaces 226, 232 of the housing 204 can form a planar surface.

In some embodiments, the sterile cover 200 can be disposable. The sterile cover 200 can be single use. The sterile cover 200 can maximize reuse of surgical navigation device 300 while maintaining sterility. The sterile cover 200 can be manufactured and packaged in a sterile state. The sterile cover 200 can provide a sterile barrier between reusable components and the outside environment. Once the surgical navigation device 300 has been used during a surgical procedure, the sterile cover 200 can be discarded and the surgical navigation device 300 can be reused. The sterile cover 200 can be a single use sterile barrier separating non-sterile surgical navigation device 300 from the sterile field. The sterile cover 200 can be intended for use with one single patient. The sterile cover 200 can be intended for use with one single procedure. The sterile cover 200 can be provided to the user in sterile medical device packaging. The sterile cover 200 can be provided to the user with instructions for single use.

In some embodiments, the sterile cover 200 can be reusable. The sterile cover 200 can be configured for use with multiple patients. The sterile cover 200 can be configured for use with multiple procedures. The sterile cover 200 can be sterilized after use. The sterile cover 200 can be sterilized using a variety of methods including thermal sterilization such as moist heat including high-pressure steam or dry heat, radiation, liquid chemical solutions including glutaraldehydes or formaldehydes, gas such ethylene oxide gas (ETO), vaporized hydrogen peroxide, ozone, UV chambers, chlorine dioxide gas, vaporized peracetic acid, and nitrogen dioxide gas, and combinations of methods thereof. The sterile cover 200 can be autoclaved. The sterile cover 200 can be subjected to high temperatures and pressure in order to sterilize the sterile cover 200 for reuse. The sterile cover 200 can be subjected to any sterilization method. The sterile cover 200 can be sterilized on site, at the hospital where the surgical procedure takes place. The sterile cover 200 can be sterilized off-site. The sterile cover 200 can comprise multiple materials such as polymers and metals. The sterile cover 200 can have multiple layers. The sterile cover 200 can be sterilized using methods that effectively sterilizes and does not damage the sterile cover 200. The sterile cover 200 can be provided to the user in sterile medical device packaging after sterilization. The sterile cover 200 can be provided to the user with instructions for multiple uses. The sterile cover 200 can be provided to the user with instructions for sterilization.

The housing 204 can receive the surgical navigation device 300 when the lid 202 is open. The surgical navigation device 300 can be received within the housing 204 without the surgical navigation device 300 contacting any outside surface of the housing 204, thereby maintaining the sterility.

The housing 204 can include one or more alignment features 250. The alignment features 250 can be projections. The alignment features 250 can be grooves. The alignment features 250 can be positioned along one or more interior walls of the housing 204. The rear housing 216 can include one or more alignment features 250 (e.g., one alignment feature, two alignment features, three alignment features, four alignment features, five alignment features, six alignment features, or any range of two of the foregoing values). The alignment features 250 can be evenly spaced. The alignment features 250 can be unevenly spaced. The alignment features 250 can be spaced apart to receive features of the surgical navigation device 300 within the housing 204. The alignment features 250 can include longitudinally oriented projections. The alignment features 250 can extend the entire length of the housing 204. The alignment features 250 can extend a portion of the length of the housing 204. The alignment features 250 can extend from the top surface 226 of the rear housing 216. The alignment features 250 can be monolithically or unitarily formed with a portion of the housing 204. The alignment features 250 can be monolithically or unitarily formed with the rear housing 216.

The alignment features 250 can allow enable the surgical navigation device 300 to be positioned within the housing 204. The alignment features 250 can reduce a gap between the housing 204 and the surgical navigation device 300. The housing 204 can include rounded edges. The housing 204 can be tapered to allow easy, low precision insertion of the surgical navigation device 300.

The housing 204 can include one or more angled ramps 252 (e.g., one ramp, two ramps, three ramps, four ramps, five ramps, six ramps, or any range of two of the foregoing values). The angled ramps 252 can be positioned along one or more interior walls of the housing 204. The rear housing 216 can include the angled ramps 252. The angled ramps 252 can be monolithically or unitarily formed with a portion of the housing 204. The angled ramps 252 can be monolithically or unitarily formed with the rear housing 216. The angled ramps 252 can extend from the alignment features 250. In some embodiments, the angled ramps 252 can be spaced apart from the alignment features 250.

The angled ramps 252 can allow enable the surgical navigation device 300 to be positioned within the housing 204. The angled ramps 252 can allow enable the surgical navigation device 300 to be positioned within the housing 204 in orientation toward the transparent layer 210. The housing 204 can facilitate secure positioning of the surgical navigation device 300 within the housing 204.

There is positional and rotational fixation applied to surgical navigation device 300 for sensor accuracy. The surgical navigation device 300 can be positioned within the sterile cover 200 to prevent or limit movement of the surgical navigation device 300 relative to the sterile cover 200. The surgical navigation device 300 can be positioned within the sterile cover 200 to prevent or limit longitudinal movement of the surgical navigation device 300 relative to the sterile cover 200. The surgical navigation device 300 can be positioned within the sterile cover 200 to prevent or limit side to side movement of the surgical navigation device 300 relative to the sterile cover 200. The surgical navigation device 300 can be positioned within the sterile cover 200 to prevent or limit rotational movement of the surgical navigation device 300 relative to the sterile cover 200. The surgical navigation device 300 can be positioned within the sterile cover 200 with minimal tolerances. The surgical navigation device 300 can be positioned within the sterile cover 200 with minimal gaps. The sterile cover 200 can be in contact with one or more sides of the surgical navigation device 300. The sterile cover 200 can be in contact with a front and back of the surgical navigation device 300. The sterile cover 200 can be in contact with sides of the surgical navigation device 300. The sterile cover 200 have several points of contact with the surgical navigation device 300.

The surgical navigation device 300 can be inserted into the sterile cover 200 in a single orientation. The sterile cover 200 can receive the surgical navigation device 300 in a single orientation. The alignment features 250 can prevent insertion of the surgical navigation device 300 in the incorrection orientation. The alignment features 250 can abut the surgical navigation device 300 in incorrect orientations, preventing the surgical navigation device 300 from being inserted into the housing 204. The alignment features 250 can extend along a shaped back surface of the surgical navigation device 300. The surgical navigation device 300 has a single position within the sterile cover 200.

The sterile cover 200 can allow attachment to a jig, as described herein. The sterile cover 200 can include one or more magnets 212. The magnets 212 can couple to a bracket of the jig, as described herein. The bracket of the jig can include compatible magnets. There are compatible magnet and bracket features for the sterile cover 200.

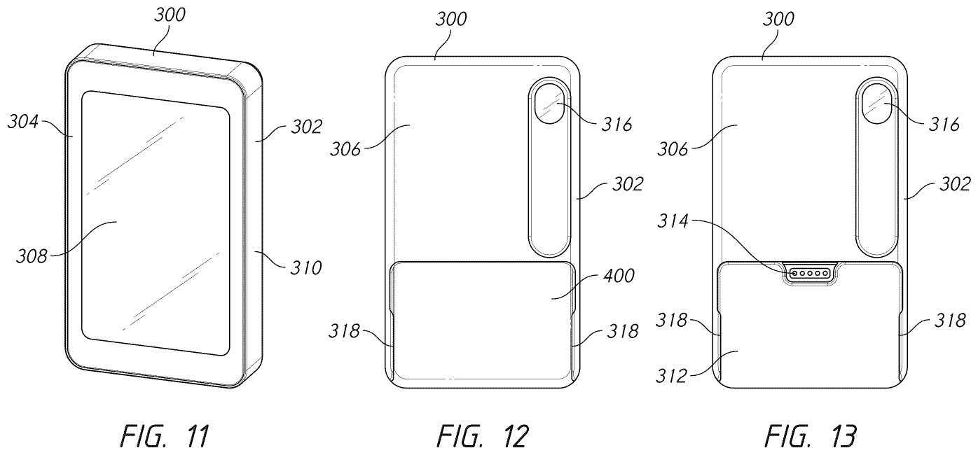

FIG. 11 is front perspective view of the surgical navigation device 300. FIG. 12 is back view of the surgical navigation device 300. FIG. 13 is another back view of the surgical navigation device 300.

The surgical navigation device 300 can be used for verifying an alignment of an orthopedic fixture or fixtures, or a cutting plane or planes, or a prosthetic component, during one or more joint replacement procedures. The surgical navigation device 300 can comprise a compact, generally hand-held, portable device for use in the joint replacement procedure. The surgical navigation device 300 can be used to locate a portion of the mechanical axis that extends through the lower tibia, or a portion thereof. The surgical navigation device 300 can be used to locate a portion of the mechanical axis that extends through the femur, or a portion thereof. The surgical navigation device 300 can be used to locate one, two, or more planes intersecting the mechanical axis. The surgical navigation device 300 can be used to orient a prosthetic component during a procedure. The surgical navigation device 300 can be used to locate one, two, or more axes relative to planes. The surgical navigation device 300 can be used to identify one, two, or more angles. The surgical navigation device 300 can be used alone or in conjunction with other devices, components, and/or systems.

The surgical navigation device 300 can comprise a generally rectangular-shaped, box-like structure having an outer housing 302. The surgical navigation device 300 can be portable. The outer housing 302 can be comprised, at least in part, of plastic including but not limited to ABS, polycarbonate, or other suitable material. The surgical navigation device 300 can be configured for hand-held use.

The surgical navigation device 300 can include a front 304 and a back 306. The front 304, or a portion of the front 304, of the surgical navigation device 300 can comprise a display 308. The display 308 can be a separate component from the outer housing 302 or can be integrated on or within the outer housing 302. The display 308 can comprise an output device such as a visual display. For example, the display 308 can comprise a liquid crystal display (“LCD”) or Ferroelectric Liquid Crystal on Silicon (“FLCOS”) display screen. The display 308 can be sized such that a user can readily read numbers, lettering, and/or symbols on the display 308 while performing a medical procedure. In some embodiments, the display 308 comprises a Quarter Video Graphics Array (“QVGA”) Thin Film Transistor (“TFT”) LCD screen. Other types of displays 308 can also be used, as can other shapes, sizes, and locations for the display 308 on the surgical navigation device 300.

The surgical navigation device 300 can comprise a touchscreen. The display 308 can detect touch input from a user. The display 308 can comprise an input device such as a touch panel. The user can provide input through simple gestures by touching the display 308, or a component that overlies the display 308. The surgical navigation device 300 can comprise a plurality of touchscreen buttons. The buttons can be activated, for example, by a finger, hand, and/or instrument to select a mode or modes of operation of the surgical navigation device 300. The surgical navigation device 300 can include the display 308 which a user can interact during a procedure.

The surgical navigation device 300 can comprise a box like structure with sides 310. The sides 310 can be rounded. The surgical navigation device 300 can form an enclosure to house internal components, as described herein. In some embodiments, the edges of the surgical navigation device 300 can be rounded. The edges connected to the back 306 can be rounded. The edges connected to the front 204 can be sharper edges.

FIGS. 12 and 13 show a back 306 of the surgical navigation device 300. The back 306 can include an attachment groove 312. The back 306 can include an electrical interface 314. The back 306 can include a power button 316. The surgical navigation device 300 can be nonsymmetric. The power button 316 can be offset to one side of the surgical navigation device 300. The power button 316 can project from the back 306 of the surgical navigation device 300.

The surgical navigation device 300 can receive the battery pack 400. The battery pack 400 can fit within the attachment groove 312 of the surgical navigation device 300. The attachment groove 312 can include projections 318 that can extend partially, or entirely, along the sides of the attachment groove 312. The projections 318 can receive corresponding, or mating, structures from the battery pack 400, so as to couple the battery pack 400 to the surgical navigation device 300. The battery pack 400 can include a complementary electrical interface configured to couple with the electrical interface 314.

The surgical navigation device 300 can comprise an electrical system. The electrical system can include an electronic control unit that communicates with one or more sensors, the battery pack 400, the display 308, and memory, among other features. The electronic control unit can receive input from the one or more sensors and the memory and transmits output to the display 308. The electronic control unit can be configured to receive and send data, as well as perform calculations based on data. In some embodiments, the electronic control unit can be configured to convert the data from a machine-readable format to a human readable format for presentation on the display 308. The electronic control unit can include one or more processors, program logic, or other substrate configurations representing data and instructions. The electronic control unit can include controller circuitry, processor circuitry, processors, general purpose single-chip or multi-chip microprocessors, digital signal processors, embedded microprocessors, and/or microcontrollers. The electronic control unit can have conventional address lines, conventional data lines, and one or more conventional control lines. In some embodiments, the electronic control unit comprises an application-specific integrated circuit (ASIC) or one or more modules configured to execute on one or more processors.

The electronic control unit can communicate with memory to retrieve and/or store data and/or program instructions for software and/or hardware. The memory can include random access memory (“RAM”), such as static RAM, for temporary storage of information and/or read only memory (“ROM”), such as flash memory, for more permanent storage of information.

The one or more sensors can be configured to provide continuous real-time data to the electronic control unit of the surgical navigation device 300. The electronic control unit can be configured to receive the real-time data from the one or more sensors and to use the data from the one or more sensors to determine, estimate, and/or calculate an orientation or position of the surgical navigation device 300. The orientation or position of the surgical navigation device 300 can be used to provide feedback to a user during the performance of a surgical procedure, such as a joint replacement surgery.

The one or more sensors can include at least one orientation sensor configured to provide real-time data to the electronic control unit related to the motion, orientation, and/or position of the surgical navigation device 300. The one or more sensors can include a gyroscopic sensor, an accelerometer sensor, a tilt sensor, a magnetometer, and/or one or more sensors and/or other similar device or devices configured to measure, and/or facilitate determination of, an orientation of the surgical navigation device 300. The one or more sensors can be configured to provide measurements relative to a reference point(s), line(s), plane(s), and/or gravitational zero. Gravitational zero, as referred to herein, refers generally to an orientation in which an axis of the sensor is perpendicular to the force of gravity, and thereby experiences no angular offset, for example tilt, pitch, roll, or yaw, relative to a gravitational force vector. The one or more sensors can be configured to provide measurements for use in dead reckoning or inertial navigation systems.

The one or more sensors can be inertial sensors. The one or more sensors can measure acceleration and angular velocity of the surgical navigation device 300. The one or more sensors can measure specific forces. The one or more sensors can motion with respect to a reference frame. In some embodiments, the one or more sensors can be prone to accumulated errors. In some embodiments, measurement errors are accumulated over time since the electronic control unit continually integrating data. In some embodiments, there is positional and rotational fixation applied to surgical navigation device 300 for sensor accuracy. The surgical navigation device 300 can be fixed relative to the sterile cover 200. The surgical navigation device 300 can be fixed relative to the bracket of the jig, as described herein. The surgical navigation device 300 can be fixed to reduce accumulated errors.

The one or more sensors can include one or more accelerometers that measure the static acceleration of the surgical navigation device 300 due to gravity. For example, the accelerometers can be used as tilt sensors to detect rotation of the surgical navigation device 300 about one or more of its axes. The one or more accelerometers can comprise a dual axis accelerometer, which can measure rotation about two axes of rotation, or a three-axis accelerometer, which can measure rotation about three axes of rotation. The changes in orientation about the axes of the accelerometers can be determined relative to gravitational zero and/or to a reference plane and/or to a reference frame registered or determined during a procedure.

The one or more sensors can include a multi-axis accelerometer which detects changes in orientation about two axes of rotation. For example, the multi-axis accelerometer can detect changes in angular position from a horizontal plane (e.g., anterior/posterior rotation) of the surgical navigation device 300 and changes in angular position from a vertical plane (e.g., roll rotation) of the surgical navigation device 300. The changes in angular position from the horizontal and vertical planes of the surgical navigation device 300 (as measured by the sensor can also be used to determine changes in a medial-lateral orientation (e.g., varus/valgus rotation) of the surgical navigation device 300.

The one or more sensors can include at least one single- or multi-axis gyroscope sensor and at least one single- or multi-axis accelerometer sensor. For example, the one or more sensors can comprise a three-axis gyroscope sensor (or three gyroscope sensors) and a three-axis accelerometer (or three accelerometer sensors) to provide positional and orientational measurements for all six degrees of freedom of the surgical navigation device 300. In some embodiments, the sensors provide an inertial navigation or dead reckoning system to continuously calculate the position, orientation, and velocity of the surgical navigation device 300 without the need for external references.

The one or more sensors can include one or more accelerometers and at least one magnetometer. The magnetometer can be configured to measure a strength and/or direction of one or more magnetic fields in the vicinity of the surgical navigation device 300. The magnetometer can advantageously be configured to detect changes in angular position about a horizontal plane. The one or more sensors can include one or more sensors capable of determining distance measurements. For example, a sensor located in the surgical navigation device 300 can be in electrical communication (wired or wireless) with an emitter element mounted at the end of a measurement probe. In certain embodiments, the electrical control unit can be configured to determine the distance between the sensor and emitter (for example, an axial length of a measurement probe corresponding to a distance to an anatomical landmark, such as a malleolus).

The one or more sensors can include a temperature sensor to monitor system temperature of the electrical system. Operation of some of the electrical components can be affected by changes in temperature. The temperature sensor can be configured to transmit signals to the electronic control unit to take appropriate action. In addition, monitoring the system temperature can be used to prevent overheating. The one or more sensors can further include temperature, ultrasonic and/or pressure sensors for measuring properties of biological tissue and other materials used in the practice of medicine or surgery, including determining the hardness, rigidity, and/or density of materials, and/or determining the flow and/or viscosity of substances in the materials, and/or determining the temperature of tissues or substances within materials.

The one or more sensors can facilitate determination of an orientation of the surgical navigation device 300 relative to a reference point, axis, plane, orientation, or frame. The reference point, axis, plane, orientation, or frame can be established during a surgical procedure. The reference point, axis, plane, orientation, or frame can be established pre-operatively, for instance based on imaging data. The reference point, axis, plane, orientation, or frame can be determined based on gravity. The reference point, axis, plane, orientation, or frame can be determined based on anatomical landmarks.

The one or more sensors can form a component of a sensor module that comprises at least one sensor, signal conditioning circuitry, and an analog-to-digital converter (“ADC”), among other features. The one or more sensors can be mounted on a stand-alone circuit board that is physically separate from, but in electrical communication with, the circuit boards containing the other electrical components described herein. The one or more sensors can be physically integrated on the circuit boards with the other electrical components. The signal conditioning circuitry of the one or more sensors can comprise one or more circuit components configured to condition, or manipulate, the output signals from the one or more sensors. In certain embodiments, the signal conditioning circuitry comprises filtering circuitry and gain circuitry. The filtering circuitry can comprise one more filters, such as a low pass filter. For example, a low pass filter can be used to remove vibrational noise or other low frequency components of the sensor output signals. The gain circuitry can comprise one or more operational amplifier circuits that can be used to amplify the sensor output signals to increase the resolution potential of the sensor.

The ADC of the sensor module can be configured to convert the analog output voltage signals of the one or more sensors to digital data samples. In some embodiments, the digital data samples comprise voltage counts. The ADC can be mounted in close proximity to the sensor to enhance signal to noise performance. The electronic control unit can include an on-board ADC that can be used to convert the sensor output signals into digital data counts.

The battery pack 400 can include one or more power sources configured to supply power to the electronic system of the surgical navigation device 300. The battery pack 400 can include one or more rechargeable or replaceable batteries. The battery pack 400 can include one or more capacitive storage devices, for example, one or more capacitors or ultracapacitors. In some embodiments, power can be supplied by other wired and/or wireless power sources. The battery pack 400 can include one or more AA alkaline, lithium, or rechargeable NiMH batteries. The surgical navigation device 300 can also include a DC/DC converter to boost the DC power from the power supply to a fixed, constant DC voltage output to the electronic control unit. The electronic control unit can be configured to monitor the battery level of the battery pack 400. Monitoring the battery level can advantageously provide advance notice of power loss. In some embodiments, the surgical navigation device 300 can comprise a timer configured to cause the surgical navigation device 300 to temporarily power off after a predetermined period of inactivity and/or to permanently power off after a predetermined time-out period. The surgical navigation device 300 can include the power button 316 that can be manipulated by the user to power on and power off the surgical navigation device 300. The power button 316 can be manipulated by the user when the surgical navigation device 300 is within the sterile cover 200.

The battery pack 400 can include one or more processors, program logic, or other substrate configurations representing data and instructions. The battery pack 400 can include controller circuitry, processor circuitry, processors, general purpose single-chip or multi-chip microprocessors, digital signal processors, embedded microprocessors, and/or microcontrollers. The battery pack 400 can have conventional address lines, conventional data lines, and one or more conventional control lines. In some embodiments, the battery pack 400 comprises an application-specific integrated circuit (ASIC) or one or more modules configured to execute on one or more processors.

The battery pack 400 can include memory. The battery pack 400 can include one or more circuits. The battery pack 400 can include one or more processors. The battery pack 400 can include a communication module to communicate with the surgical navigation device 300. The battery pack 400 can include an authentication integrated circuit. The battery pack 400 can include a co-processor. The battery pack 400 can include one or more algorithms including Secure Hash Algorithm. The battery pack 400 can include a Secure Hash Algorithm engine. The battery pack 400 can include a rewritable memory, such as EEPROM. The battery pack 400 can include non-volatile memory. The battery pack 400 can include a stored secret key. The battery pack 400 can compute a message authentication code (MAC). The battery pack 400 can include a counter. The battery pack 400 can generate data related to number of procedures. The battery pack 400 can generate data related to the type of license. The battery pack 400 can generate data related to authentication. The battery pack 400 can generate data related to licensing.

The battery pack 400 can supply power to the display 308. The display 308 can comprise an LCD display. The electronic system can include a display controller and/or an LED driver and one or more LEDs to provide backlighting for the display 308. For example, the display controller can comprise an LCD controller integrated circuit. The electronic control unit can be configured to control the LED driver via a pulse width modulation port to control the brightness of the LED display. For example, the LED driver can drive four LEDs spaced around the display screen to provide adequate backlighting to enhance visibility. The display 308 can be configured to display one or more on-screen graphics. The on-screen graphics can comprise graphical user interface (“GUI”) images or icons. The GUI images can include instructions and/or instructive images, such as illustrated surgical procedure steps. The GUI images can include visual indicators of the orientation information received from the one or more sensors. For example, the display 308 can be configured to display degrees and either a positive or negative sign to indicate direction of rotation from a reference plane. For example, the display can be configured to display a bubble level indicator to aid a user in maintaining a particular orientation. The display 308 can also be configured to display alphanumeric text, symbols, and/or arrows.

The display 308 can comprise a touchscreen display. The display 308 can allow the user to enter data, make selections, input instructions or commands to the surgical navigation device 300. The display 308 can allow the user to verify a position or orientation of the surgical navigation device 300. The display 308 can provide a warning to the user when a particular condition occurs. The display 308 can provide status or notifications, for example, to indicate low battery level, an error condition, etc. In some embodiments, output from the surgical navigation device 300 can be sent to external display devices, data storage devices, servers, and/or other computing devices (e.g., via a wireless network communication link).

The battery pack 400 can supply power to the one or more sensors. The one or more sensors can comprise one or more accelerometers. Accelerometers can measure the static acceleration of gravity in one or more axes to measure changes in tilt orientation. For example, a three-axis accelerometer can measure the static acceleration due to gravity along three orthogonal axes. A two-axis accelerometer can measure the static acceleration due to gravity along two orthogonal axes. The output signals of an accelerometer can comprise analog voltage signals. The output voltage signals for each axis can fluctuate based on the fluctuation in static acceleration as the accelerometer changes its orientation with respect to the gravitational force vector. The acceleration along each axis can be independent of the acceleration along the other axis or axes.

Multi-axis accelerometers can be conceptualized as having a separate accelerometer sensor for each of its axes of measurement, with each sensor responding to changes in static acceleration in one plane. In certain embodiments, each accelerometer sensor is most responsive to changes in tilt (i.e., operates with maximum or optimum accuracy and/or resolution) when its sensitive axis is substantially perpendicular to the force of gravity (i.e., when the longitudinal plane of the accelerometer sensor is parallel to the force of gravity) and least responsive when the sensitive axis is parallel to the force of gravity (i.e., when the longitudinal plane of the accelerometer sensor is perpendicular to the force of gravity). The tilt sensitivity diminishes between −90 degrees and −45 degrees and between +45 degrees and +90 degrees. This resolution problem at the outer ranges of tilt motion makes the measurements much less accurate for tilt measurements over 45 degrees. In certain embodiments, the mounting angle of the surgical navigation device 300 relative to the jig is known and the one or more sensors can be mounted to be offset at an angle such that the accelerometer sensors can operate in their more accurate, steeper slope regions. For example, for use during the surgery procedures described herein, the one or more sensors can be mounted at approximately a 22-degree angle relative to the anterior-posterior axis of the surgical navigation device 300 to account for a predetermined range of motion of the surgical navigation device 300 during the procedures. It should be appreciated by one of ordinary skill in the art that the accelerometer can be mounted at acute angles other than approximately 22 degrees. In some embodiments, the one or more sensors can be mounted to be offset to account for a predetermined range of motion about other axes of rotation as well. In some embodiments, for example, when a three-axis accelerometer is used, the accelerometer sensor(s) can be mounted in parallel with the anterior-posterior axis of the surgical navigation device 300. In one three-axis accelerometer arrangement, a handoff system can be incorporated to ensure that the accelerometer sensors with the most accurate reading (e.g., <45 degrees) are being used at each orientation position. The handoff system can employ hysteresis to avoid “bouncing” phenomena during the handoffs between the accelerometer sensors.

The surgical navigation device 300 can comprise one or more circuit boards and/or other circuitry capable of installation within the surgical navigation device 300. The surgical navigation device 300 can include a sensor board and a main board. In some embodiments, the components of the sensor module including the one or more sensors can be mounted on the sensor board and the other components of the electrical system are mounted on the main board. In some embodiments, the sensor board and the main board can be combined into a single circuit board. The sensor board and the main board can comprise rigid or flexible circuit boards. The sensor board and the main board can be fixedly or removably attached to the outer housing 302. The surgical navigation device 300 can communicate with the battery pack 400. The surgical navigation device 300 can include a communication module. The surgical navigation device 300 and the battery pack 400 can include a wired connection for communication and power. The surgical navigation device 300 and the battery pack 400 with a wired half-duplex serial bus, such as 1-wire®. The communication can be asynchronous. The communication can be over a single data line. The battery pack 400 can communicate with a co-processor on the surgical navigation device 300. The authentication result is communicated to the co-processor on the surgical navigation device 300.

The sensor board can be mounted at an approximately 22-degree angle relative to a plane extending longitudinally through the housing 302, which can be parallel to or correspond to an anterior-posterior axis of the main board. As described above, mounting the sensor board at an offset angle can enable the one or more sensors to operate in the regions of maximum or optimum sensitivity, accuracy and/or resolution. The particular mounting offset angle can be selected based on a range of motion of the surgical navigation device 300 during a particular orthopedic procedure. For example, during the tibial preparation procedures, the surgical navigation device 300 can be aligned with the coronal plane of the tibia with the leg in flexion and during the femoral preparation procedures described herein, the surgical navigation device 300 can be aligned to the leg in extension. Accordingly, the mounting offset angle is set at approximately 22 degrees to keep the orientation of the sensors from getting too close to the less accurate, low-resolution range when the surgical navigation device 300 is positioned in the two flexion/extension orientations.

There can be a conversion process for converting an analog voltage output signal of a multi-axis accelerometer into an angle degree measurement for presentation on the display 308. The steps can be implemented with hardware and/or software. For each axis of rotation measured (e.g., pitch and roll), the multi-axis accelerometer can continuously output an analog voltage signal. The signal conditioning circuitry of the sensor module can filter the analog output voltage signal (e.g., with a low pass filter) to remove noise from the signal that may be present due to the high sensitivity of the multi-axis accelerometer. The signal conditioning circuitry amplifies, or boosts, the output voltage signal, for example, via the gain circuitry described above. The ADC can convert the continuous analog voltage signal into a discrete digital sequence of data samples, or voltage counts. In some embodiments, the ADC can sample the analog voltage signal once every two milliseconds; however, other sampling rates are possible. In some embodiments, the analog voltage signal is oversampled. The electronic control unit can generate a stable data point to be converted to an angle measurement. The electronic control unit can apply a median filter to the sampled data to eliminate outliers (e.g., spikes) in the data. The electronic control unit can use a finite impulse response (“FIR”), or an infinite impulse response (“IIR”) filter implemented in a software module. The electronic control unit can convert the voltage count data to an angle measurement in degrees. In performing the conversion, the electronic control unit can be configured to apply a calibration conversion algorithm based on a calibration routine performed during a testing phase prior to sale of the surgical navigation device 300. The calibration conversion can be configured to account for unit-to-unit variations in components and sensor placement. The calibration routine can be performed for each axis being monitored by the multi-axis accelerometer. The calibration conversion can comprise removing any mechanical or electrical offsets and applying an appropriate gain calibration for a positive or negative tilt.

The tilt angle for each axis (e.g., pitch and roll) of the multi-axis accelerometer can be calculated from the voltage count data based on standard trigonometric relationships as the arcsin of the acceleration component in each particular axis. In arrangements in which the electronic control unit applies the calibration conversion, the tilt angle for each axis can be calculated. In arrangements where a dual-axis accelerometer is used, the electronic control unit can be configured to adjust the pitch angle (x axis) calculation to account for the mounting offset angle of the dual-axis accelerometer relative to the outer housing 302 of the surgical navigation device 300. The result is an absolute angle for each axis of rotation (e.g., pitch, roll) being monitored by the dual-axis accelerometer. The absolute pitch and roll angles can be used to calculate orientation measurements of the surgical navigation device 300, such as a flexion-extension angle and a varus/valgus angle for a knee procedure, and/or an anteversion angle and an inclination angle for hip procedure. Orientation measurements for the surgical navigation device 300 can be determined based on a wide variety of reference frames in conjunction with any of a variety of surgical procedures.

The one or more sensors can be configured to measure changes in angular position from a horizontal axis (e.g., pitch) and a vertical axis (e.g., roll). In performing the methods described herein, the surgical navigation device 300 can be mounted such that the pitch measurement of the sensor corresponds to rotation about the x axis (e.g., flexion/extension rotation) of the reference frame and such that the roll measurement of the sensor corresponds with rotation about the y axis of the reference frame.

The calculations can be performed by software modules executed by the electronic control unit. In some embodiments, other calculations can be derived based on the type of sensor or sensors used, the procedure being performed, and/or the reference frame being employed. In some embodiments, the electronic control unit can perform a stabilization routine, process, or algorithm to assess or determine the stability, or reliability, of the calculated angle measurements. In some embodiments, the software modules on the surgical navigation device 300 and/or the battery pack 400 can provide authentication. In some embodiments, the software modules on the surgical navigation device 300 and/or the battery pack 400 can provide licensing.

The surgical navigation device 300 can be reusable. In some embodiments, the surgical navigation device 300 can have the same surgical workflow and capabilities as a single use surgical navigation device. The surgical navigation device 300 can include a removable battery pack 400. The surgical navigation device 300 and the battery pack 400 can be provided as separate components. The surgical navigation device 300 and the battery pack 400 can be coupled at the beginning of the joint replacement procedure. The surgical navigation device 300 can be reprocessed via cleaning and disinfection. The surgical navigation device 300 is not sterile. The surgical navigation device 300 can be used repeatedly. The surgical navigation device 300 can have a useful life limited by a programmed in usage counter. The battery pack 400 can be single use. The battery pack 400 can include the usage counter to ensure that the battery pack 400 is only used once. The battery pack 400 can provide authentication and license management. The battery pack 400 can be used for one type of surgical procedure. The battery pack 400 can be licensed for use only with knee joint replacement procedures. The battery pack 400 can be licensed for use only with hip joint replacement procedures. The user can select between two or more types of batteries packs 400 depending on the type of surgery.

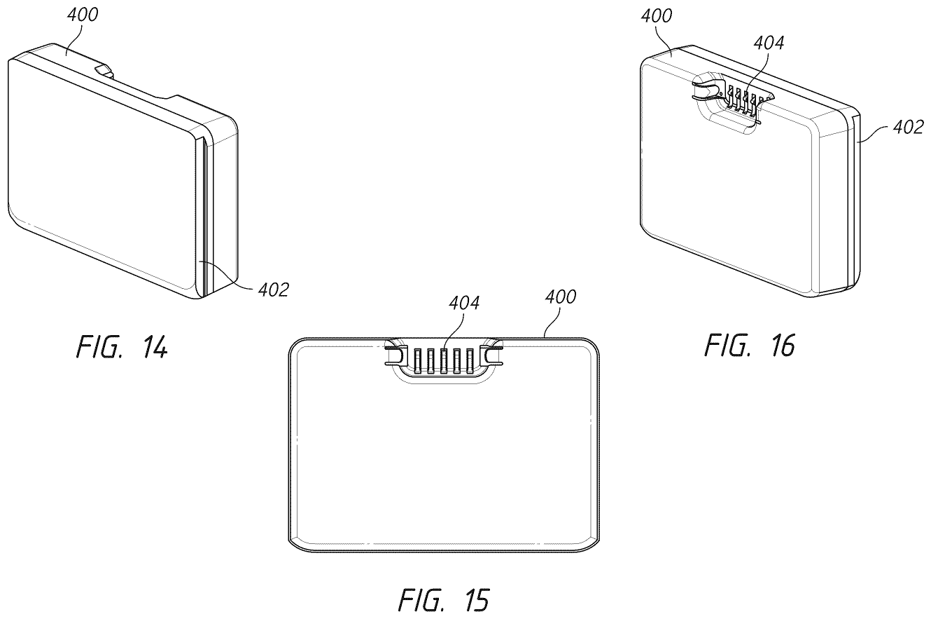

FIG. 14 is front perspective view of the battery pack 400. FIG. 15 is back view of the battery pack 400. FIG. 16 is back perspective view of the battery pack 400. The battery pack 400 can be single use. The battery pack 400 can be disposable. The battery pack 400 can be for power delivery. The battery pack 400 can be for authentication security. The battery pack 400 can be for license management.

The battery pack 400 can fit within the attachment groove 312 of surgical navigation device 300. The battery pack 400 can include grooves 402 that can extend partially, or entirely, along the sides of the battery pack 400. The projections 318 of the surgical navigation device 300 can be received by the corresponding grooves 402 from the battery pack 400, so as to couple the battery pack 400 to the surgical navigation device 300. The battery pack 400 can slide upward into engagement with the attachment groove 312 of surgical navigation device 300.

The battery pack 400 can include a complementary electrical interface 404. The complementary electrical interface 404 is configured to couple with the electrical interface 314 of the surgical navigation device 300. The battery pack 400 can provide electrical power through the electrical interface 404. The battery pack 400 can supply power to the electronic control unit. The battery pack 400 can supply power to the display 308. The battery pack 400 can supply power to the one or more sensors. The battery pack 400 can supply power to any feature of the surgical navigation device 300. The battery pack 400 can provide signals through the electrical interface 404.

The battery pack 400 can be used for authentication. The battery pack 400 can be used for security. The surgical navigation device 300 can be a portable device that requires a portable power supply. There may be counterfeiters that want to supply cheap replacement batteries, which may not have the features required by the original manufacturer of the surgical navigation device 300. For instance, the battery pack 400 may have mechanical safety features. For instance, the battery pack 400 may have electrical safety features.

The battery pack 400 and the surgical navigation device 300 mate via a physical connection. The surgical navigation device 300 can receive a battery pack 400. The battery pack 400 can fit within the attachment groove 312. The battery pack 400 can provide power to the surgical navigation device 300 via internal batteries. The physical connection alone may not ensure that the battery pay 400 is authentic.

The battery pack 400 can provide authentication signals to the surgical navigation device 300. The battery pack 400 can provide licensing signals to the surgical navigation device 300. The battery pack 400 can communicate with a co-processor integrated circuit (IC) on the surgical navigation device 300 to provide authentication and licensing. The battery pack 400 can communicate over a 1-Wire® bus.

The battery pack 400 can provide authentication. The battery pack 400 can include an authentication integrated circuit. The surgical navigation device 300 can include co-processor. The battery pack 400 can communicate with the co-processor to provide authentication. The authentication integrated circuit and co-processor can include a Secure Hash Algorithm engine, such as a SHA-3 engine. The authentication integrated circuit and co-processor can include electrically erasable programmable read-only memory (EEPROM). The secure hash algorithm utilizes a stored secret key on both the authentication integrated circuit and co-processor to compute a Keyed-Hash Message Authentication Code (HMAC). The HMAC is used to verify authenticity of the battery pack 400. The authentication result is communicated from the co-processor to the main processor of the surgical navigation device 300. The battery pack 400 and the surgical navigation device 300 can use a message authentication code including a cryptographic hash function. The battery pack 400 and the surgical navigation device 300 can use a message authentication code including a secret cryptographic key. The battery pack 400 and the surgical navigation device 300 can provide authentication using a shared secret key.

The battery pack 400 can provide licensing. The authentication integrated circuit of the battery pack 400 can include a decrement counter that is used to ensure the battery pack 400 can only be used for one surgical procedure. The battery pack 400 can be licensed for a single procedure. The authentication integrated circuit of the battery pack 400 can include data stored in EEPROM that provides the type of license, for instance knee procedure or hip procedure. The battery pack 400 can be licensed for a single type of procedure. The battery pack 400 can be licensed for a knee procedure. The battery pack 400 can be licensed for a hip procedure. The user can be provided with a battery pack 400 for each procedure. The user can be provided with a battery pack 400 for each type of procedure.

In some embodiments, an electrical identification system is used to authenticate the battery pack 400. In some embodiments, the battery pack 400 can include an integrated circuit. In some embodiments, the surgical navigation device 300 can include a co-processor. In some embodiments, the surgical navigation device 300 can function as the challenger. The surgical navigation device 300 can send a command to receive data from the battery pack 400. The data can include the surgical procedure code. The data can include an identification code. The data can include the number of surgical cycles. The data from the battery pack is compared with data in the surgical navigation device 300. In some embodiments, the surgical navigation device 300 and the battery pack 400 can have a fixed response to a fixed challenge. The surgical navigation device 300 allows operation if the data is valid. The surgical navigation device 300 does not allow operation if the data is invalid. The surgical navigation device 300 can provide an error message on the display 308 if the data is invalid. The surgical navigation device 300 can send and receive signals through the electrical interface 314 of the surgical navigation device 300. The battery pack 400 can send and receive signals through the electrical interface 404 of the battery pack 400.

In some embodiments, a more secure electrical identification system is utilized. The surgical navigation device 300 and the battery pack 400 can have a challenge and response scheme. The surgical navigation device 300 can send a random challenge to the battery pack 400. The random challenge includes data generated by the surgical navigation device 300. The battery pack 400 receive the random challenge and calculates a response based on a key. The surgical navigation device 300 also calculates a response based on the key. The battery pack 400 and the surgical navigation device 300 can both contain a Secure Hash Algorithm engine and EEPROM. The battery pack 400 and the surgical navigation device 300 can both generate a response. The secure hash algorithm utilizes a stored secret key on both the battery pack 400 and the surgical navigation device 300. The battery pack 400 and the surgical navigation device 300 both compute a Keyed-Hash Message Authentication Code (HMAC). The Keyed-Hash Message Authentication Code is used to verify authenticity of the battery pack 400. The authentication result can be communicated from between processors of the surgical navigation device 300. The surgical navigation device 300 and the battery pack 400 can have a random challenge. The challenge and response can change every time. The surgical navigation device 300 does not allow operation if the data is invalid. The surgical navigation device 300 can provide an error message on the display 308 if the data is invalid.

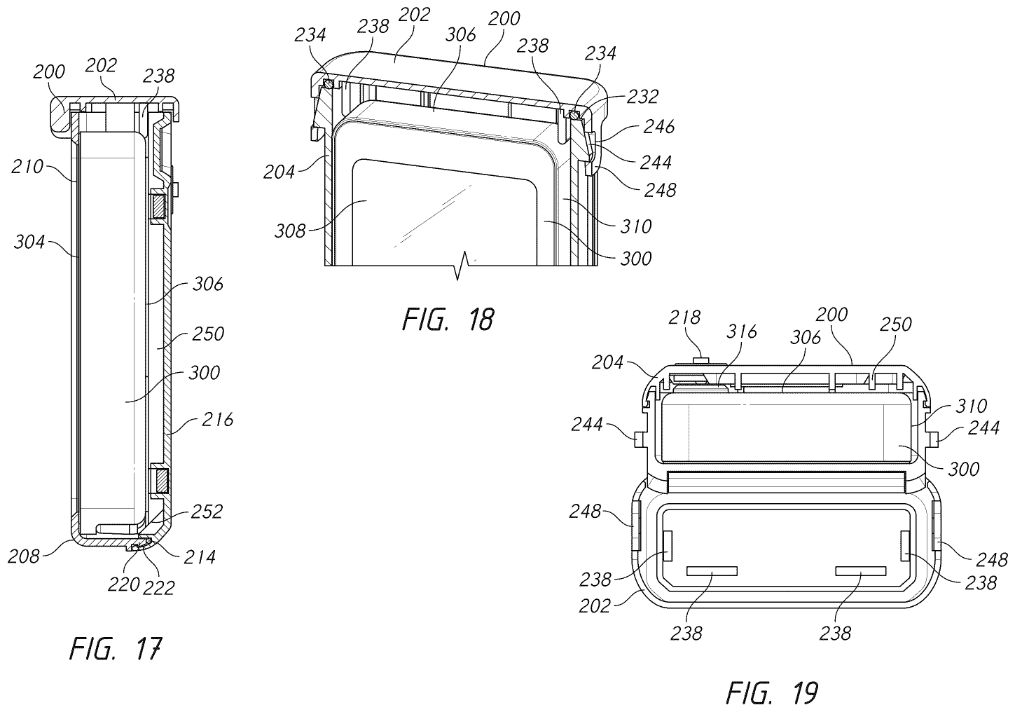

FIG. 17 is a cross sectional view of the sterile cover 200 and the surgical navigation device 300. FIG. 18 is another cross-sectional view of the sterile cover 200 and the surgical navigation device 300. FIG. 19 is a top view of the sterile cover 200 and the surgical navigation device 300.

The front housing 208 and the rear housing 216 can be fixed. The front housing 208 and the rear housing 216 can form a permanent connection. The front housing 208 can include the interlocking projection 220. The rear housing 216 can include the interlocking groove 222. The housing seal gasket 214 can be disposed between the front housing 208 and the rear housing 216. The housing seal gasket 214 can form a seal between the front housing 208 and the rear housing 216. The front housing 208 and the rear housing 216 can form a watertight seal. The front housing 208 and the rear housing 216 can be permanently coupled. The housing 204 can be provided to the user as permanently coupled.

In some embodiments, the sterile cover 200 can be provided to the user with the lid 202 opened. In some embodiments, the sterile cover 200 can be provided to the user with the lid 202 closed. The user can open the lid 202 while maintain the sterility of the sterile cover 202. The lid 202 can be closed, as shown in FIG. 17. The lid 202 can include the lid seal gasket 234. The lid seal gasket 234 can be disposed between the housing 204 and the lid 202. The lid seal gasket 234 can form a liquid resistant seal between the housing 204 and the lid 202 when the lid 202 is closed. The lid 202 and the housing 204 can form a watertight seal. The lid 202 compresses the lid seal gasket 234 against the top surface 232 of the front housing 208 and the top surface 226 of the rear housing 216 when the lid 202 is closed. The lid 202 and the housing 204 can be pivotally coupled to allow the lid 202 to open and close. The sterile cover 200 can be provided to the user with the lid 202 coupled to the housing 204. In some embodiments, the pivotal connection between the lid 202 and the housing 204 is configured to be permanent. The pivotal connection between the lid 202 and the housing 204 is configured to resist disassembly during use.

The lid 202 can be closed. The lid 202 can be closed by connecting a latch. The front housing 208 can include two projections 244. The lid 202 can include two grooves 246 of the tabs 248. The projections 244 and the grooves 246 can be pushed together to lock. The user can manually pull the tabs 248 outward to release the lock. The lid 202 closing relative to the housing 204 is reversable. The lid 202 and the housing 204 is configured to limit inadvertent disassembly during use. For instance, the tabs 248 may require significant force to release the grooves 246 from the projections 244. For instance, the tabs 248 may require significant force to release the lid 202. For instance, two or more tabs 248 may need to be actuated to release the lid 202. The plurality of tabs 248 may limit inadvertent disassembly of the lid 202 during use. The position of tabs 248 may limit inadvertent disassembly of the lid 202 during use. The strength of the connection between the grooves 246 from the projections 244 may limit inadvertent disassembly of the lid 202 during use.

The alignment projections 238 of the lid 202 can facilitate alignment of the surgical navigation device 300 within the housing 204. The alignment projections 238 of the lid 202 can be internal ribs. The alignment projections 238 of the lid 202 can provide a mechanism that helps a user avoid mistakes by preventing, correcting, or drawing attention to human errors as they occur. The alignment projections 238 of the lid 202 can allow the surgical navigation device 300 to be inserted in a single orientation within the housing 204 in order to allow the lid 202 to close. The alignment projections 238 of the lid 202 can prevent incorrect orientation of the surgical navigation device 300 within the sterile cover 200.

The alignment projections 238 of the lid 202 can be flexible fingers. The alignment projections 238 of the lid 202 can provide a force on the surgical navigation device 300 in the y direction, or downward. The alignment projections 238 of the lid 202 can provide a force on the surgical navigation device 300 in the x direction, or frontward. The alignment projections 238 of the lid 202 can provide a force to push the surgical navigation device 300 downward and frontward. The alignment projections 238 of the lid 202 can provide an interference fit. The alignment projections 238 of the lid 202 can provide press fit against rounded edges of the surgical navigation device 300. The alignment projections 238 of the lid 202 can provide press fit against the back 306 of the surgical navigation device 300. The alignment projections 238 of the lid 202 can provide press fit against the sides 310 of the surgical navigation device 300. The alignment projections 238 of the lid 202 can drive the surgical navigation device 300 down and forward when the lid 202 closes. The alignment projections 238 can fill gaps between the surgical navigation device 300 and the sterile cover 200.

The alignment projections 238 of the lid 202 can prevent the incorrect orientation of the surgical navigation device 300. The alignment projections 238 of the lid 202 can press against the sides 310 and back 306 of the surgical navigation devices 300. In some embodiments, the back 306 of the surgical navigation device 300 is rounded and the front 304 of the surgical navigation device 300 is flat. The alignment projections 238 of the lid 202 can abut curved edges of the surgical navigation device 300. The alignment projections 238 of the lid 202 can abut curved edges along the sides 310 and back 306 of the surgical navigation device 300. In some embodiments, the lid 202 cannot close if the alignment projections 238 abut the front 304 of the surgical navigation device 300. In some embodiments, the lid 202 only closes if the surgical navigation device 300 faces toward the front of the housing 204. In some embodiments, the lid 202 only closes if the surgical navigation device 300 is in a single orientation. In some embodiments, the lid 202 only closes if the display 308 of the surgical navigation device 300 faces toward transparent layer 210. In some embodiment, the lid 202 cannot close if the alignment projections 238 contact the front 304 of the surgical navigation device 300. In some embodiment, the lid 202 can close if the alignment projections 238 contact the sides 310 and back 306 of the surgical navigation device 300.

The housing 204 can include one or more alignment features 250. The alignment features 250 can include longitudinally oriented projections. The rear housing 216 can include alignment features 250. The alignment features 250 can position the surgical navigation device 300 forward. The alignment features 250 can position the surgical navigation device 300 toward the front of the housing 204. The alignment features 250 can position the surgical navigation device 300 toward transparent layer 210. The alignment features 250 can be asymmetric. The alignment features 250 can include at least one alignment feature 250 that prevents the surgical navigation device 300 from being inserted into the housing 204 in the incorrect orientation. The alignment features 250 can include at least one alignment feature 250 that would abut the power button 316 of the surgical navigation device 300 if the surgical navigation device 300 were incorrectly inserted into the housing 204. The power button 316 aligns with the soft touch button 218 when the surgical navigation device 300 is properly inserted within the sterile cover 200.

The housing 204 can include one or more angled ramps 252. The surgical navigation device 300 can rest on the angled ramps 252. The angled ramps 252 can drive the surgical navigation device 300 toward the transparent layer 210 of the sterile cover 200. The rear housing 216 can include the angled ramps 252. The angled ramps 252 of the rear housing 216 can provide a force on the surgical navigation device 300 in the y direction, or upward. The angled ramps 252 of the rear housing 216 can provide a force on the surgical navigation device 300 in the x direction, or frontward. The angled ramps 252 of the rear housing 216 can provide a force to push the surgical navigation device 300 upward and frontward. The angled ramps 252 of the rear housing 216 can drive the surgical navigation device 300 upward and forward when the lid 202 closes.

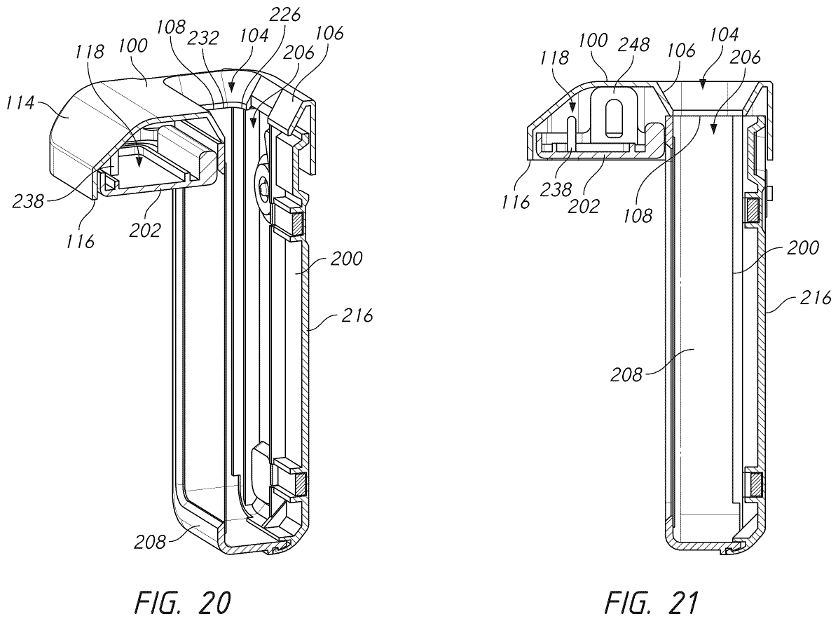

FIG. 20 is a cross-sectional view of the transfer funnel of 100 and the sterile cover 200. FIG. 21 is another cross-sectional view of the transfer funnel 100 and the sterile cover 200. The transfer funnel 100 can limit or prevent accidental contact between sterile and non-sterile components during aseptic transfer. The transfer funnel 100 can be sterile and the sterile cover 200 can be sterile.

The transfer funnel 100 can include the ledge 108. The ledge 108 can be formed by the tapered edges 106. The ledge 108 can extend around the lumen 104. The ledge 108 can function as a stop against the sterile cover 200. The ledge 108 can function as a stop against the housing 204. The ledge 108 can rest against the top surface 226 of the rear housing 216. The ledge 108 can rest against the top surface 232 of the front housing 208. The ledge 108 can rest against the sterile cover 200. The ledge 108 can form a flat surface for abutting the sterile cover 200. The ledge 108 can form a flat surface for abutting the housing 204. The transfer funnel 100 does not extend into the opening 206 of the housing 204. The transfer funnel 100 abuts the top surfaces 226, 232 of the housing 204. The transfer funnel 100 does not extend into the sterile cover 200. The transfer funnel 100 can be entirely outside of the sterile cover 200. The transfer funnel 100 can remain external to the housing 204. The transfer funnel 100 can be supported by the top surfaces 226, 232 of the housing 204. The transfer funnel 100 can extend along the sides of the sterile cover 200. The transfer funnel 100 can extend along at least three sides of the housing 204. The transfer funnel 100 can extend along the rear housing 216. The transfer funnel 100 can extend along the sides of the front housing 208. The transfer funnel 100 can extend along the sides of the lid 202. The transfer funnel 100 can extend along the front of the lid 202.

The transfer funnel 100 can include the lumen 104. The sterile cover 200 can include the opening 206. The lumen 104 of the transfer funnel 100 and the opening 206 of the sterile cover 200 can be continuous. The lumen 104 of the transfer funnel 100 and the opening 206 of the sterile cover 200 can be coaxial. The lumen 104 of the transfer funnel 100 and the opening 206 of the sterile cover 200 can be aligned. The lumen 104 of the transfer funnel 100 can be offset from the neutral center of the transfer funnel 100. The neutral center of the transfer funnel 100 can be offset from the lumen 104. The neutral center of the transfer funnel 100 can be aligned with a portion of the lid 202. The transfer funnel 100 can be supported at multiple points of engagement with the sterile cover 200. The points of engagement can be external to the housing 204. The points of engagement can include the top surfaces 226, 232 of the housing 204. The points of engagement can include engagement with the opened lid 202.

The transfer funnel 100 can include the extension 114, the lower edge 116 and the cavity 118. The extension 114 can extend over top of the lid 202 when the lid 202 is open. The extension 114 can extend along at least three sides of the lid 202 when the lid 202 is open. The transfer funnel 100 does not extend underneath the lid 202. The extension 114 can cover the lid 202 when the lid 202 is open. The extension 114 can cover the lid 202 when the ledge 108 abuts the top surfaces 226, 232 of the housing 204. The lower edge 116 can extend below the lid 202. The cavity 118 can receive the lid 202. The cavity 118 can receive the tabs 248 of the lid 202. The tabs 248 of the lid 202 can provide additional points of engagement between the transfer funnel 100 and the sterile cover 200. The tabs 248 of the lid 202 can facilitate stabilization of the transfer funnel 100 relative to the sterile cover 200. The tabs 248 of the lid 202 can abut an internal surface of the transfer funnel 100. The tabs 248 of the lid 202 can abut the extension 114 when the transfer funnel 100 is positioned relative to the sterile cover 200. The transfer funnel 100 can be supported by tabs 248 of the lid 202. The transfer funnel 100 can be supported by the opened lid 202. The cavity 118 can receive the alignment projections 238. The transfer funnel 100 can be lowered relative to the sterile cover 200. The transfer funnel 100 can be raised to remove the transfer funnel 100. The transfer funnel 100 can have a single orientation relative to the sterile cover 200 when the transfer funnel 100 is engaged with the sterile cover 200.

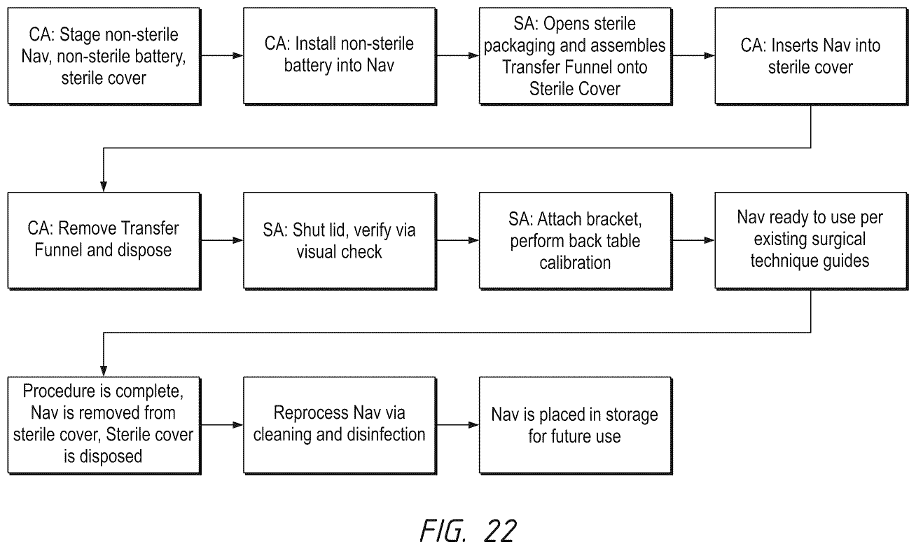

FIG. 22 is a method of use. The transfer funnel 100 can be used in any method of use described herein. The sterile cover 200 can be used in any method of use described herein. The surgical navigation device 300 can be used in any method of use described herein. The battery pack 400 can be used in any method of use described herein.

The method can include staging one or more components. The method can include staging the non-sterile surgical navigation device 300. The method can include staging the non-sterile battery pack 400. The method can include staging the sterile cover 200. The sterile cover 200 can remain within sterile packaging. The transfer funnel 100 can remain within sterile packaging. The staging can be performed by the circulating assistant. The staging can be performed by the non-sterile assistant.

The method can include installing the non-sterile battery pack 400 into the surgical navigation device 300. Referring to FIGS. 11-16, the battery pack 400 can slide within the attachment groove 312 of the surgical navigation device 300. The surgical navigation device 300 can receive the mating structures from the battery pack 400, so as to couple the battery pack 400 to the surgical navigation device 300. When installed, the electrical interface 404 of the battery pack 400 couples with the electrical interface 314 of the surgical navigation device 300. The battery pack 400 can provide licensing and authentication. The installing the non-sterile battery pack 400 into the surgical navigation device 300 can be performed by the circulating assistant. The installing the non-sterile battery pack 400 into the surgical navigation device 300 can be performed by the non-sterile assistant.

The method can include opening the sterile packaging. The opening can be performed by the sterile assistant. The method can include opening the lid 202 if the lid is closed. Referring to FIGS. 9 and 10, the lid 202 can include two pins 240 aligned along the axis of rotation. The front housing 208 can include two sockets 242 aligned along the axis of rotation. The sockets 242 can receive the pins 240 to form a hinge joint. The lid 202 can swing open. The lid 202 can pivot relative to the housing 204. The method can include assembling the transfer funnel 100 onto the sterile cover 200. The transfer funnel 100 can be lowered onto the opened lid 202 and the housing 204. The transfer funnel 100 can rest against the opened lid 202. The transfer funnel 100 can rest against the housing 204. The assembling can be performed by the sterile assistant.

The method can include inserting the surgical navigation device 300 into the sterile cover 200. The method can include inserting the surgical navigation device 300 through the transfer funnel 100. The method can include inserting the surgical navigation device 300 through the lumen 104 of the transfer funnel 100. The method can include inserting the surgical navigation device 300 into the housing 204. The method can include inserting the surgical navigation device 300 through the opening 206 of the housing 204. The inserting of the surgical navigation device 300 can be performed by the circulating assistant. The inserting of the surgical navigation device 300 can be performed by the non-sterile assistant.

The method can include removing the transfer funnel 100. The method can include disposing of the transfer funnel 100. The removing and disposing of the transfer funnel 100 can be performed by the circulating assistant. The removing and disposing of the transfer funnel 100 can be performed by the non-sterile assistant.

The method can include shutting the lid 202. The method can include verifying that the lid 202 is shut via a visual check. The shutting of the lid 202 can be performed by the sterile assistant.

The method can include attaching the bracket. The attaching of the bracket can be performed by the sterile assistant. The method can include performing back table calibration of the surgical navigation device 300. The calibration can be performed by the sterile assistant.

The method can include surgical navigation. The surgical navigation device 300 can be ready to use per surgical technique guides. The surgical navigation device 300 can be used in a joint replacement procedure. The surgical navigation device 300 can be used for knee replacement methods. The surgical navigation device 300 can be used for hip replacement methods.

The method can include completing the procedure. The method can include removing the surgical navigation device 300 from the sterile cover 200. The method can include disposing the sterile cover 200. The method can include removing the battery pack 400. The method can include disposing the battery pack 400. The method can include disposing of the battery pack 400 after a single procedure.

The method can include reprocessing the surgical navigation device 300. The method can include cleaning the surgical navigation device 300. The method can include disinfecting the surgical navigation device 300.

The method can include storing the surgical navigation device 300. The surgical navigation device 300 can be placed in storage for future use. The method can include repeating one or more steps. The method can include repeating one or more steps with a new sterile cover 100, a new transfer funnel 100, and a new battery pack 400, while reusing the surgical navigation device 300.

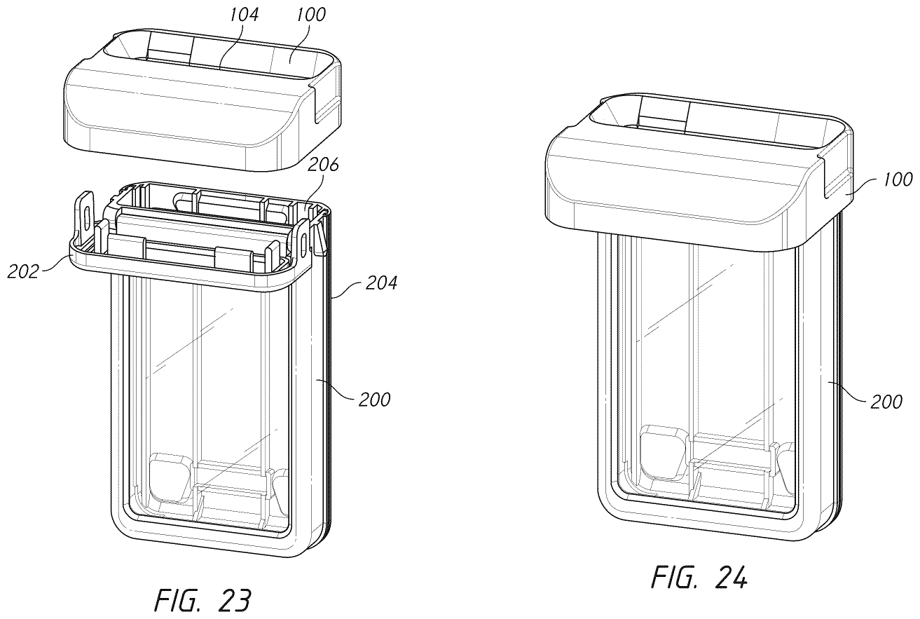

FIG. 23 is a method of use of the transfer funnel 100 and the sterile cover 200. FIG. 24 is a method of use of the transfer funnel 100 and the sterile cover 200. The transfer funnel 100 can be lowered relative to the sterile cover 200. Referring FIG. 4, the transfer funnel 100 can include the ledge 108. The transfer funnel 100 can include the lower edge 116. The transfer funnel 100 can include the cavity 118. The transfer funnel 100 can include a lumen 104. Referring to FIG. 10, the housing 204 can form a flat surface when the lid 202 is opened. The rear housing 216 can include the top surface 226. The top surface 226 can be flat. The front housing 208 can include a top surface 232. The top surface 232 can be flat. The top surfaces 226, 232 can be aligned to form a flat surface.

The housing 204 can include the opening 206. The ledge 108 of the transfer funnel 100 can rest against the top surface 226, 232 of the housing 204. The ledge 108 of the transfer funnel 100 can abut the housing 204 of the sterile cover 200. The transfer funnel 100 does not extend into the opening 206 of the housing 204. The transfer funnel 100 can be entirely external to the housing 204. The opening 206 of the housing 204 can align with the lumen 104 of the transfer funnel 104. The cavity 118 of the transfer funnel 100 can receive the lid 202. The lid 202 can be within the transfer funnel 100 when the transfer funnel 100 is positioned relative to the sterile cover 200. In some embodiments, the transfer funnel 100 extends below the lid 202. The transfer funnel 100 can prevent accidental contact between the surgical navigation device 300 and the lid 202 when the transfer funnel 100 is positioned. The transfer funnel 100 can prevent accidental contact between the surgical navigation device 300 and the housing 204 when the transfer funnel 100 is positioned. The transfer funnel 100 can prevent accidental contact between the surgical navigation device 300 and the sterile cover 200 when the transfer funnel 100 is positioned. The transfer funnel 100 provides a sterile barrier between the surgical navigation device 300 and the sterile cover 200 during insertion of the surgical navigation device 300 into the housing 204.

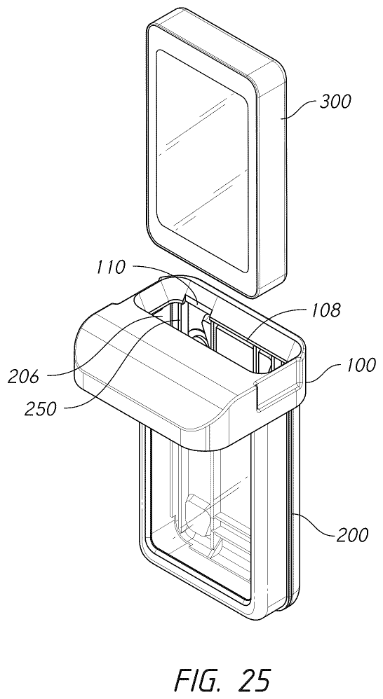

FIG. 25 is a method of use of the transfer funnel 100, the sterile cover 200, and the surgical navigation device 300. The transfer funnel 100 can include the alignment slot 110. The alignment slot 110 can ensure that the surgical navigation device 300 is inserted into the transfer funnel 100 correctly. The alignment slot 110 can accommodate the power button 316 of the surgical navigation device 300. The power button 316 of the surgical navigation device 300 can slide relative to the alignment slot 110. In some embodiment, the surgical navigation device 300 can only be inserted into the housing 204 of the sterile cover 200 if the power button 316 is aligned with the alignment slot 110 of the transfer funnel 100. In some embodiments, the transfer funnel 100 ensures that the surgical navigation device 300 is properly oriented for insertion into the sterile cover 200.

The housing 204 can include one or more alignment features 250. The alignment features 250 can be projections. The alignment features 250 can be positioned along one or more interior walls of the housing 204. In some embodiment, the surgical navigation device 300 can only be inserted into the housing 204 of the sterile cover 200 if the power button 316 is aligned between the alignment features 250. In some embodiment, the surgical navigation device 300 can only be inserted into the housing 204 of the sterile cover 200 in one orientation relative to the alignment features 250. In some embodiments, the alignment features 250 of the sterile cover 200 ensures that the surgical navigation device 300 is properly oriented for insertion into the sterile cover 200.

The battery pack 400 can be installed with the surgical navigation device 300 before inserting the surgical navigation device 300 into the transfer funnel 100. The battery pack 400 can be installed with the surgical navigation device 300 before inserting the surgical navigation device 300 into the sterile cover 200. The battery pack 400 can provide authentication security. The battery pack 400 can provide license management. The battery pack 400 can provide power to the surgical navigation device 300 via internal batteries. The battery pack 400 can have one or more processors. The battery pack 400 can include one or more integrated circuits. The battery pack 400 can include the Secure Hash Algorithm (SHA-3) engine. The battery pack 400 can include memory such as Electrically Erasable Programmable Read-Only Memory (EEPROM). The secure hash algorithm utilizes a stored secret key on both the authentication integrated circuit and co-processor. The authentication integrated circuit and co-processor both compute a Keyed-Hash Message Authentication Code (HMAC). The HMAC is used to verify authenticity of the battery pack 400. The authentication result is communicated from the battery pack 400 to the surgical navigation device 300.

The authentication integrated circuit of the battery pack 400 can include a counter. The counter can be used to ensure the battery pack 400 can only be used for one surgical procedure. The authentication integrated circuit of the battery pack 400 can contain data stored in memory that provides the type of license. The battery pack 400 can be licensed for use for one surgical procedure. The battery pack 400 can be licensed for use for one type of surgical procedure, such as a knee procedure or a hip procedure.

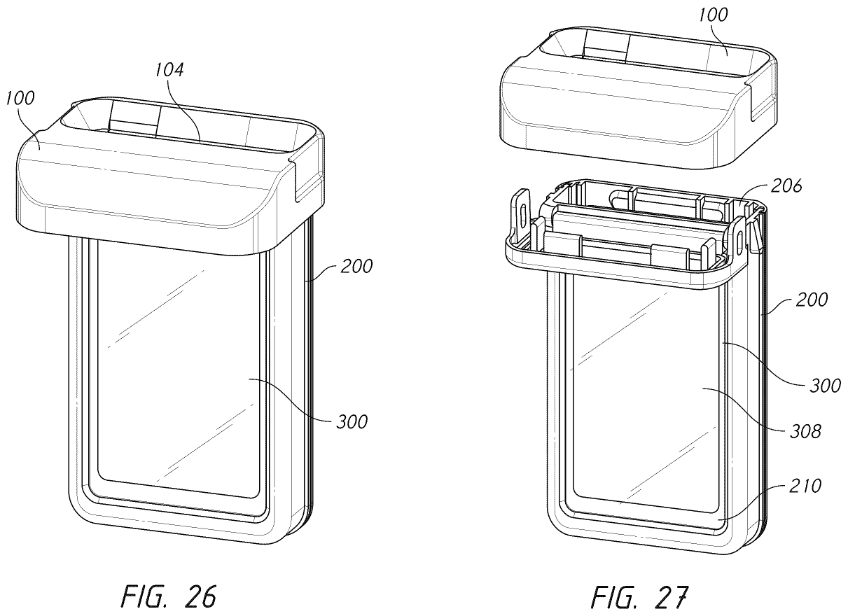

FIG. 26 is a method of use of the transfer funnel 100, the sterile cover 200, and the surgical navigation device 300. FIG. 27 is a method of use of the transfer funnel 100, the sterile cover 200, and the surgical navigation device 300.

The surgical navigation device 300 can be inserted into the sterile cover 200. The surgical navigation device 300 can be inserted entirely through the lumen 104 of the transfer funnel 100. The surgical navigation device 300 can be inserted entirely through the opening 206 of the sterile cover 200. The surgical navigation device 300 can be seated within the housing 204. The surgical navigation device 300 can be seated against the one or more alignment features 250. The surgical navigation device 300 can be seated against one or more angled ramps 252. The transfer funnel 100 can be removed. The display 308 of the surgical navigation device 300 can be viewed through the transparent layer 210 of the sterile housing 200. The display 308 of the surgical navigation device 300 can be used as a touchscreen through the transparent layer 210 of the sterile housing 200. The surgical navigation device 300 is positioned such that the display 308 is visible through the transparent layer 210 of the housing 204. The transparent layer 210 can overlie the display 308.

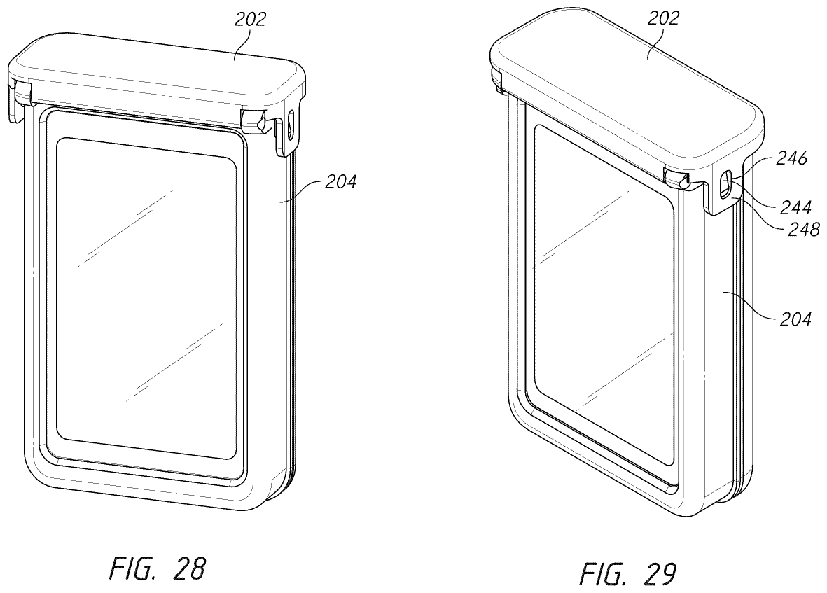

FIG. 28 is a method of use of the sterile cover 200 and the surgical navigation device 300. FIG. 29 is a method of use of the sterile cover 200 and the surgical navigation device 300.

The lid 202 can be closed relative to the housing 204 of the sterile cover 200. The lid 202 can be closed after the transfer funnel 100 is removed. The lid 202 can be closed after the surgical navigation device 300 is inserted into the housing 204. The housing 204 can include one or more projections 244. The lid 202 can include one or more grooves 246. The lid 202 can pivot relative to the housing 204 to close. The one or more grooves 246 can receive the one or more projections 244. The lid 202 and the housing 204 can be pushed together to lock. The one or more projections 244 and the one or more grooves 246 are configured to be reversable upon the user applying a force to the one or more tabs 248 of the lid 202. The one or more tabs 248 can be pushed outward by the user to disengage the one or more projections 244 and the one or more grooves 246. In some embodiments, the user must use two hands to disengage the lid 202. In some embodiments, the user must use considerable force to disengage the lid 202. The one or more projections 244 and the one or more grooves 246 can be designed to firmly and securely lock. The one or more projections 244 and the one or more grooves 246 can be designed such that intentional and forceful movement is needed to disengage the lid 202.

Referring to FIGS. 17 and 18, the sterile cover 200 can include features to provide a liquid resistant seal. The lid 202 can include the lid seal gasket 234. The lid seal gasket 234 can facilitate the seal between the lid 202 and the housing 204. The housing 204 can include the housing seal gasket 214. The housing seal gasket 214 can facilitate the seal between the front housing 208 and the rear housing 216.

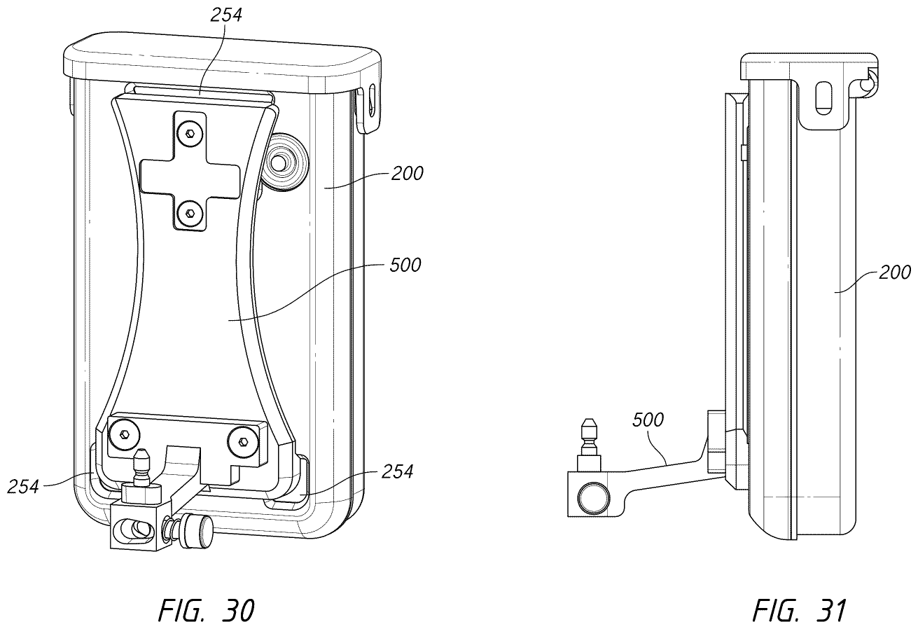

FIG. 30 is a method of use of the sterile cover 200, the surgical navigation device 300, and a bracket 500. FIG. 31 is a method of use of the sterile cover 200, the surgical navigation device 300, and the bracket 500. Referring to FIG. 8, the housing 204 can include one or more magnets 212. The magnets 212 can be spaced apart along the rear housing 216. The rear housing 216 can include two attachment grooves 228 to receive two magnets 212. The magnets 212 can couple to the bracket 500. The bracket 500 can couple to portions of a jig. The jig can be designed for a knee replacement procedure. The jig can be designed for a hip replacement procedure. The bracket 500 can include magnets configured to attract the magnets 212 of the housing 200.

The housing 204 can include the rear housing 216. The rear housing 216 can include one or more bracket alignment grooves 254. The bracket alignment grooves 254 can receive a portion of the bracket 500. The bracket alignment grooves 254 can facilitate alignment of magnets of the sterile cover 200 and magnets of the bracket 500. The sterile cover 200 can attached to the jig on an opposite surface of the sterile cover 200 as the transparent layer 210. The sterile cover 200 can attached to the jig on an opposite surface of the sterile cover 200 as the display 308 of the surgical navigation device 300.

The sterile cover 200 can couple to a surgical jig using a variety of jig attachments. The sterile cover 200 can include magnets that couple to corresponding magnets of the jig. The sterile cover 200 can include a groove that couples to corresponding projection of the jig. For example, one or more projections of the jig can be inserted into one or more grooves or channels along the back of the sterile cover 200. The sterile cover can have one or more shaped grooves that interlock with the projection of the jig. Once the projection of the jig is seated within the groove the sterile cover, the interconnection can form a rigid body that inhibits movement of the sterile cover 200, and thus the surgical navigation device 300. The projection of the jig can be biased to facilitate attachment of the jig and the sterile cover 200. The projection can be biased toward a locking position in which the projection is moved toward the groove. The coupling between the jig and the sterile cover 200 can reversible. The user can apply a force to the projection of the jig or the sterile cover 200 to release the sterile cover 200. The sterile cover 200 can include a projection that couples to corresponding groove of the jig. The sterile cover 200 can include a lever-like structure which can pivot in order to lock and unlock with the jig.

The sterile cover 200 can include an attachment structure or structures. The attachment structure or structures can be formed in the back of the sterile cover 200, such as the rear housing 216. The attachment structure or structures can extend partially or entirely along the back side of the sterile cover 200. The attachment structure or structures can be formed in the sides of the sterile cover, such as the sides of the rear housing 216 and the sides of the front housing 208. The attachment structure or structures can be formed as grooves or projections in the sterile cover 200. The attachment structure or structures can receive a corresponding or mating structure to couple or lock the sterile cover 200 to the jig. The sterile cover 200 can couple with the jig with a single point of attachment. The sterile cover 200 can couple with the jig with at least two points of contact, such as two grooves and two corresponding projections.

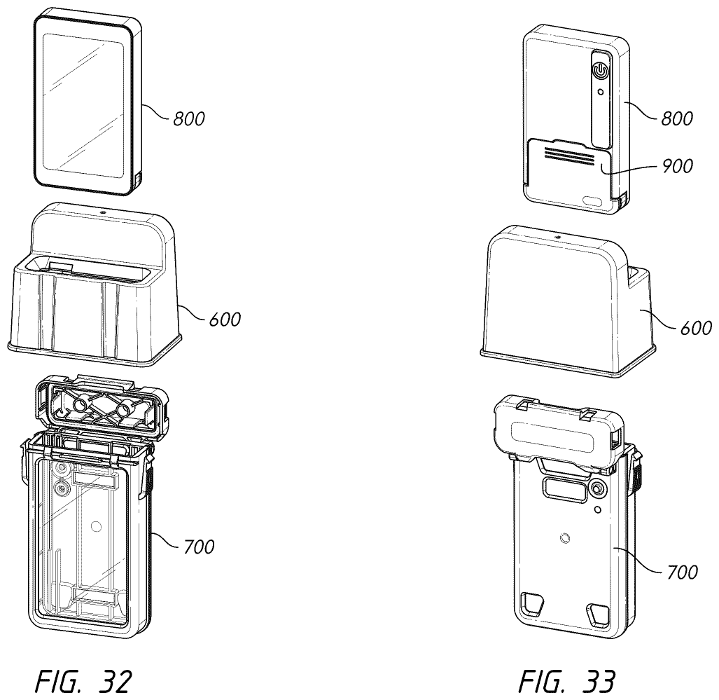

FIG. 32 is a front exploded view of a system. FIG. 33 is a back exploded view of the system. The system can include a transfer funnel 600. The transfer funnel 600 can have any feature of the transfer funnel 100 described herein. The system can include a sterile cover 700. The sterile cover 700 can have any feature of the sterile cover 200 described herein. The system can include a surgical navigation device 800. The surgical navigation device 800 can have any feature of the surgical navigation device 300 described herein. The system 600 can include a battery pack 900. The battery pack 900 can have any feature of the battery pack 400 described herein.

The system can be used in any methods described herein. The system can have any functionalities described herein. The sterile cover 700 can be designed to provide a sterile barrier for the surgical navigation device 800 during a surgical procedure. In some embodiments, the surgical navigation device 800 can be incompatible with sterilization. For instance, the surgical navigation device 800 can include electronics such as a touchscreen. The sterile cover 700 can be a cover that provides a barrier between the non-sterile surgical navigation device 800 and the surgical field. The surgical navigation device 800 can be received within the sterile cover 700 in a single orientation. The battery pack 900 can be coupled with the surgical navigation device 800 before insertion of the surgical navigation device 800 into the sterile cover 700. The surgical navigation device 800 and/or the sterile cover 700 can include features that orient the surgical navigation device 800 within the sterile cover 700. The sterile cover 700 can have one or more seals that isolate the surgical navigation device 800 from the surgical field. The sterile cover 700 can have a window that allows the touchscreen of the surgical navigation device 800 to be viewed and manipulated during the surgical procedure. The sterile cover 700 can be single use. The surgical navigation device 800 can be reusable. The surgical navigation device 800 can be reusable for a set number of surgical procedures. The surgical navigation device 800 can include one or more inertial sensors. The inertial sensors can include any feature or functionality described herein. Features of the sterile cover 700 and the surgical navigation device 800 are described in greater detail herein.

The system can include the transfer funnel 600. The transfer funnel 600 can cover a portion of the sterile cover 700 during insertion of the surgical navigation device 800 into the sterile cover 700. The transfer funnel 600 can be single use. The transfer funnel 600 can be sterile. The transfer funnel 600 can facilitate aseptic transfer. In some embodiments, the transfer funnel 600 is an optional component. In some embodiments, the sterile cover 700 is utilized without the transfer funnel 600. In some embodiments, the surgical navigation device 800 is utilized without the transfer funnel 600.

The system can be used in a joint replacement procedure, such as a knee replacement procedure or a hip replacement procedure. The surgical navigation device 800 can facilitate the positioning of a cutting block. The surgical navigation device 800 can facilitate the positioning of an acetabular cup. The surgical navigation device 800 can be utilized in any method described in U.S. application Ser. No. 13/800,620 filed Mar. 13, 2013, U.S. application Ser. No. 15/550,564 filed Aug. 11, 2014, U.S. application Ser. No. 15/920,216 filed Mar. 13, 2018, U.S. application Ser. No. 15/920,202 filed Mar. 13, 2018, all of which are incorporated by reference herein in their entirety. The system is compatible with a plurality of instrument sets. The system is compatible with instrument sets for knee replacement. The system is compatible with instrument sets for total knee replacement. The system is compatible with instrument sets for unicompartmental knee replacement. The system is compatible with instrument sets for partial knee replacement. The system is compatible with instrument sets for knee balancing. The system is compatible with instrument sets for hip replacement.

In some embodiments, the surgical navigation device 800 can be limited to a certain number of procedures. In some embodiments, the surgical navigation device 800 can be limited to ten procedures. In some embodiments, the surgical navigation device 800 can be limited to twenty procedures. In some embodiments, the surgical navigation device 800 can include a counter that tracks the number of surgical procedures performed by the surgical navigation device 800.

In some embodiments, the single use components are specific to knee procedures. In some embodiments, the single use components are specific to hip procedures. In some embodiments, the surgical navigation device 800 is agnostic to the type of joint replacement procedure. In some embodiments, the surgical navigation device 800 comprises software to perform both hip replacement and knee replacement. In some embodiments, the single use components have a shelf life of 2 years. In some embodiments, the sterile cover 700 can be single use. In some embodiments, the sterile cover 700 can be a hard case. In some embodiments, the sterile cover 700 can include a transparent touch screen cover. In some embodiments, the battery pack 900 is single use. In some embodiments, the battery pack 900 provides power. In some embodiments, the battery pack 900 provides authorization. In some embodiments, the battery pack 900 provides license management. In some embodiments, the surgical navigation device 800 is reusable. In some embodiments, the surgical navigation device 800 is non-sterile.

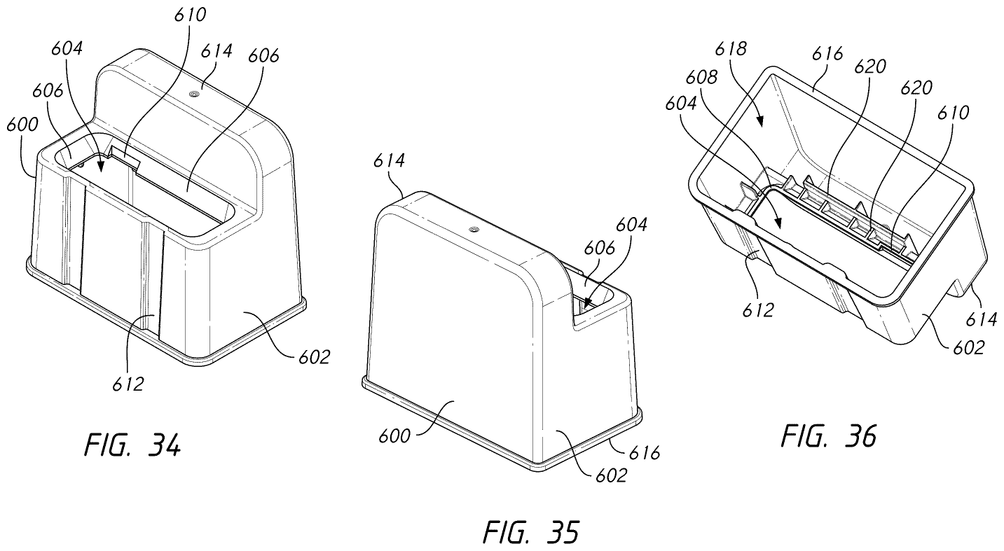

FIG. 34 is front perspective view of the transfer funnel 600. FIG. 35 is back perspective view of the transfer funnel 600. FIG. 36 is another perspective view of the transfer funnel 600. The transfer funnel 600 can include a body 602. The body 602 can be integrally formed. The body 602 can be one continuous unitary structure. The transfer funnel 600 can include a lumen 604. The lumen 604 can extend through a portion of the body 602. The lumen 604 can be sized and shaped to receive the surgical navigation device 800. The lumen 604 can extend vertically through the body 602. The transfer funnel 600 can include one or more tapered edges 606. The one or more tapered edges 606 can extend to the lumen 604. The one or more tapered edges 606 can guide the surgical navigation device 800 through the lumen 604. The one or more tapered edges 606 can have rounded corners. The one or more tapered edges 606 can be continuous. The one or more tapered edges 606 can be interrupted. The one or more tapered edges 606 form a generally rectangular opening into the lumen 604. The one or more tapered edges 606 form an elongated opening into the lumen 604.

The transfer funnel 600 can include a ledge 608. The ledge 608 can be formed by the one or more tapered edges 606. The ledge 608 can be the bottom surface of the one or more tapered edges 606. The ledge 608 can form the periphery of the lumen 604. The ledge 608 can form a surface for abutting other components. The ledge 608 can abut the sterile cover 700. The ledge 608 can function as a stop against the sterile cover 700. The ledge 608 can form a closed shape. The ledge 608 can form a perimeter. The ledge 608 can form a flat surface.

The transfer funnel 600 can include an alignment slot 610. The alignment slot 610 can guide the surgical navigation device 800 through the lumen 604. The alignment slot 610 can ensure that the surgical navigation device 800 is inserted into the lumen 604 in a single orientation. The alignment slot 610 can ensure that the surgical navigation device 800 is correctly aligned with the transfer funnel 600. The alignment slot 610 can ensure that the surgical navigation device 800 is correctly aligned with the sterile cover 700 when the transfer funnel 600 covers at least a portion of the sterile cover 700. The alignment slot 610 can be offset from the center of the lumen 604. The alignment slot 610 can extend along one or more tapered edges 606. The alignment slot 610 can extend along the ledge 608. The alignment slot 610 can allow the user to view the surgical navigation device 800 when the surgical navigation device 800 is inserted in the sterile cover 700.

The transfer funnel 600 can include one or more finger grips 612. The finger grips 612 can allow the user to raise and lower the transfer funnel 600 relative to the sterile cover 700. The finger grips 612 can be located on the front of the transfer funnel 600. The finger grips 612 can be located on the outer surface of the transfer funnel 600. In some embodiments, the transfer funnel 600 can be rigid. The transfer funnel 600 can maintain its shape during use. The transfer funnel 600 can maintain the shape of the ledge 608 during use. The transfer funnel 600 can maintain the shape of the lumen 604 during use.

The transfer funnel 600 can include an extension 614. The extension 614 can be integrally formed with the body 602. The extension 614 can extend vertically. The extension 614 can include curved edges. The lumen 604 can be side-by-side with the extension 614. The lumen 604 can be offset relative to a midline of the body 602. The extension 614 can be offset relative to a midline of the body 602. The lumen 604 can be positioned on one side of the midline of the body 602 and the extension 614 can be positioned on the other side of the midline of the body 602. The one or more tapered edges 606 can be side-by-side with the extension 614. The one or more tapered edges 606 can be offset relative to a midline of the body 602. The extension 614 can be offset relative to a midline of the body 602. The one or more tapered edges 606 can be positioned on one side of the midline of the body 602 and the extension 614 can be positioned on the other side of the midline of the body 602. The extension 614 can receive a portion of the sterile cover 700. The extension 614 can receive a portion of the sterile cover 700 when the sterile cover 700 is open to receive the surgical navigation device 800.

The transfer funnel 600 can include one or more alignment features 620. The alignment features 620 facilitate alignment of the transfer funnel 600 and the sterile cover 700. The alignment features 620 can include one more ridges that press against the sterile cover 700 when the sterile cover 700 is received within the transfer funnel 600. The alignment features 620 can include one more ridges that abut the sterile cover 700 when the sterile cover 700 is open to receive the surgical navigation device 800.

The transfer funnel 600 can include a lower edge 616. The extension 614 and the lower edge 616 can define the height of the transfer funnel 600. The transfer funnel 600 can cover a portion of the sterile cover 700 when the sterile cover 700 is received within the transfer funnel 600. In some embodiments, the transfer funnel 600 can cover approximately half of the sterile cover 700. In some embodiments, the transfer funnel 600 can cover a portion of the sterile cover 700 including 25%, 30%, 35%, 40%, 45%, 50%, more than 25%, more than 30%, more than 35%, more than 40%, more than 45%, more than 50%, less than 75%, less than 70%, less than 65%, less than 60%, less than 55%, less than 50%, or any range of two of the foregoing values. The lower edge 616 can extend around the perimeter of the body 602. The lower edge 616 can encircle the sterile cover 700 when the sterile cover 700 is received within the transfer funnel 600. The transfer funnel 600 can include a cavity 618. The cavity 618 can accommodate a portion of the sterile cover 700 when the sterile cover 700 is received within the transfer funnel 600. The cavity 618 can accommodate a portion of the sterile cover 700 when the sterile cover 700 is open to receive the surgical navigation device 800.

The transfer funnel 600 can accommodate a portion of the sterile cover 700 during aseptic transfer. The transfer funnel 600 can receive a portion of the sterile cover 700 within the cavity 618. The transfer funnel 600 can receive a portion of the sterile cover 700 within the extension 614. The transfer funnel 600 can rest upon the sterile cover 700. In some embodiments, the transfer funnel 600 does not enter the sterile cover 700. Rather, the ledge 608 abuts a surface of the sterile cover 700. The transfer funnel 600 covers an upper portion of the sterile cover 700. The transfer funnel 600 is configured to prevent contact between the non-sterile surgical navigation device 800 and the external surfaces of the sterile cover 700. The transfer funnel 600 is configured to facilitate aseptic transfer. The transfer funnel 600 is configured to facilitate maintaining the sterility of the sterile cover 700 during insertion of the surgical navigation device 800 into the sterile cover 700. The transfer funnel 600 can cover a portion of the sterile cover 700 when the sterile cover 700 is opened. The transfer funnel 600 can cover the sterile cover 700 from the top of the sterile cover 700 to a mid-portion of the sterile cover 700. The transfer funnel 600 can cover the entire sterile cover 700 when viewed from the direction of insertion of the surgical navigation device 800. The transfer funnel 600 can include the alignment slot 610. The alignment slot 610 can allow the surgical navigation device 800 to be inserted relative to the sterile cover 700 in a single orientation.

The transfer funnel 600 can be a separate component from the sterile cover 700. The transfer funnel 600 can be a separate, sterile shield. The transfer funnel 600 can be disposable. The transfer funnel 600 can be single use. The transfer funnel 600 can be manufactured and packaged in a sterile state. The transfer funnel 600 can be provided to the user with instructions for single use. The transfer funnel 600 can provide a barrier between the sterile cover 700 and the surgical navigation device 800 during insertion. The transfer funnel 600 can limit or prevent contact between the surgical navigation device 800 and outside surfaces of the sterile cover 700 during insertion of the surgical navigation device 800 into sterile cover 700. Once the surgical navigation device 800 is inserted into the sterile cover 700, the transfer funnel 600 can be vertically lifted relative to the sterile cover 700. The transfer funnel 600 can be discarded and the sterile cover 700 can be closed. The transfer funnel 600 can be a sterile barrier separating the non-sterile surgical navigation device 800 from the sterile cover 700.

In some embodiments, the transfer funnel 600 can be reusable. The transfer funnel 600 can be sterilized after use. The transfer funnel 600 can be sterilized using any method described herein. The transfer funnel 600 can be autoclaved. The transfer funnel 600 can be provided to the user with instructions for multiple uses. The transfer funnel 600 can be provided to the user with instructions for sterilization. In some embodiments, the transfer funnel 600. The surgical navigation device 800 can be inserted directly into the sterile cover 700. The surgical navigation device 800 can be inserted into the sterile cover 700 without the transfer funnel 600.

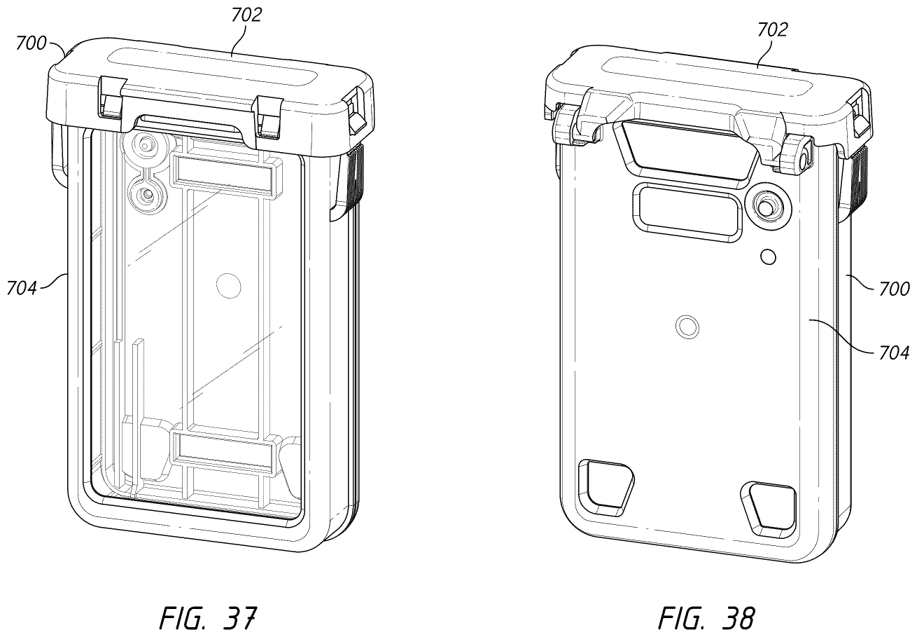

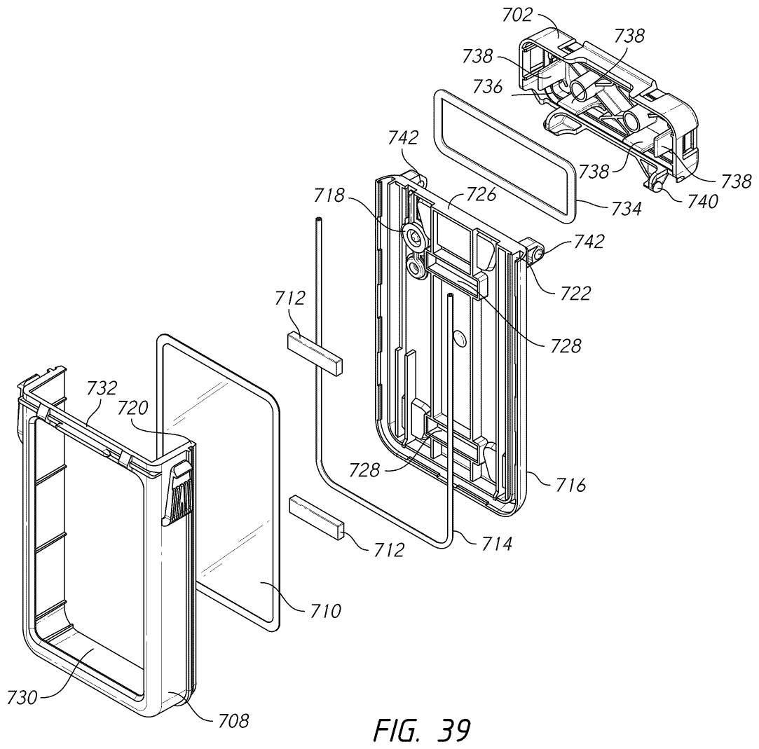

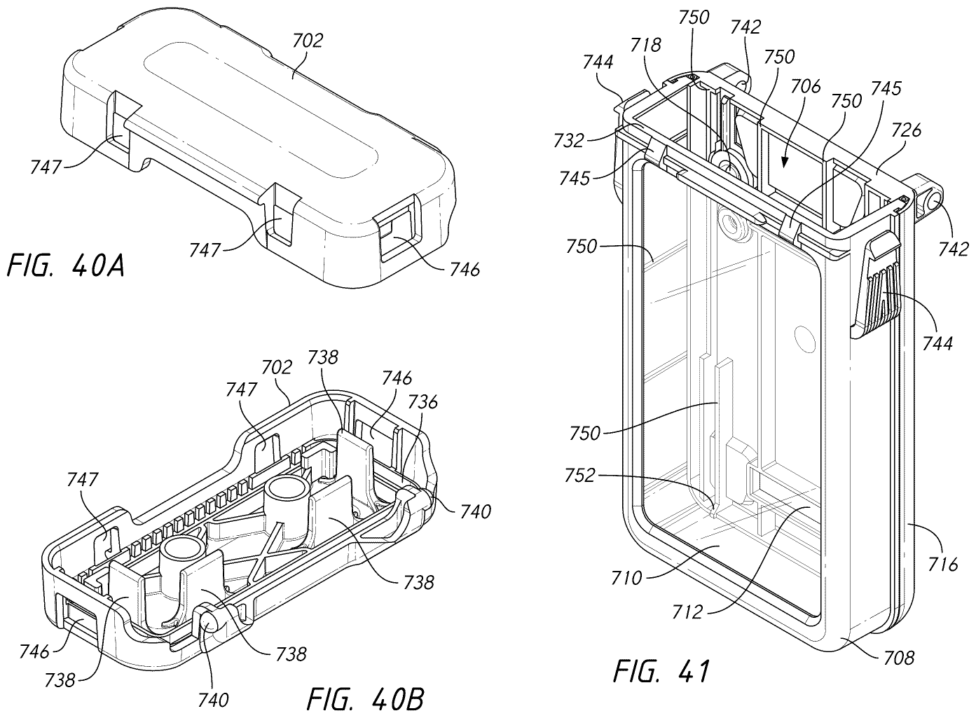

FIG. 37 is front perspective view of the sterile cover 700. FIG. 38 is back perspective view of the sterile cover 700. FIG. 39 is an exploded perspective view of the sterile cover 700. The sterile cover 700 can include a lid 702. The sterile cover 700 can include a housing 704. The lid 702 of the sterile cover 700 is configured to open and close relative to the housing 704 of the sterile cover 700. The sterile cover 700 can include a hinge on which the lid 702 swings as the lid 702 opens or closes. The sterile cover 700 can include a moveable joint or mechanism which connects the lid 702 and the housing 704. The lid 702 can be coupled to the housing 704 such that the lid 702 can open and close. The lid 702 can pivot relative to the housing 704. The lid 702 can swing open or close. The lid 702 can be opened to allow insertion and removal of the surgical navigation device 800 relative to the sterile cover 700. FIG. 40A is a top perspective view of the lid 702 of the sterile cover 700. FIG. 40B is a bottom perspective view of the lid 702 of the sterile cover 700. FIG. 41 is perspective view of the housing 704 of the sterile cover 700.

The housing 704 can including an opening 706 as shown in FIG. 41. The opening 706 can extend from a top surface of the housing 704. The opening 706 can allow access into the housing 704 when the lid 702 is opened. The lid 702 can cover the opening 706 of the housing 704 when the lid 702 is closed 702. The lid 702 can seal the housing 704 when the lid 702 is closed. The sterile cover 700 can be fully enclosed when the lid 702 is closed. The housing 704 and the lid 702 can be completely closed on all sides when the lid 702 is closed. The housing 704 and the lid 702 can fully enclose the surgical navigation device 800 when the lid 702 is closed. The housing 704 and the lid 702 can form a physical barrier around the surgical navigation device 800 when the lid 702 is closed. The surgical navigation device 800 can be contained within the sterile cover 700 when the lid 702 is closed. The surgical navigation device 800 can be bounded or confined within the sterile cover 700 when the lid 702 is closed. The surgical navigation device 800 can be completely shut in or surrounded when the lid 702 is closed.

The housing 704 can include several components. The housing 704 can include any features of the housing 204 described herein. The housing 704 can include a front housing 708. The housing 704 can include a transparent layer 710. The housing 704 can include one or more magnets 712. The housing 704 can include a housing seal gasket 714. The housing 704 can include a rear housing 716. The housing 704 can include a soft touch button 718.

The components of the housing 704 can be separately formed. The components of the housing 704 can comprise different materials. The components of the housing 704 can be coupled during manufacturing. The components of the housing 704 can be coupled using any method described herein. The components of the housing 704 can be permanently coupled. The components of the housing 704 connected such that the components cannon be easily dissembled. The front housing 708 and the rear housing 716 can be coupled by a snap fit mechanism. The front housing 708 and the rear housing 716 can have interlocking components that can be pushed together during manufacturing. The front housing 708 can include an interlocking projection 720. The rear housing 716 can include an interlocking groove 722. The interlocking projection 720 and the interlocking groove 722 can interlock to form a snap-fit.

The sterile cover 700 can include one or more features to limit or prevent leakage. The housing 704 can include one or more mechanical seals. The housing seal gasket 714 can be disposed between the front housing 708 and the rear housing 716. The housing seal gasket 714 can fill the space between two or more mating surfaces. The housing seal gasket 714 can be a deformable material designed to create a seal. The housing seal gasket 714 can form a liquid resistant seal between the front housing 708 and the rear housing 716. The housing seal gasket 714 can be disposed within the interlocking groove 722 of the rear housing 716. The front housing 708 can include the interlocking projection 720 that compresses the housing seal gasket 714. The housing seal gasket 714 can be compressed when the interlocking projection 720 and the interlocking groove 722 interlock.

The rear housing 716 can include the soft touch button 718. The soft touch button 718 can be integrally formed with the rear housing 716. The soft touch button 718 can allow the user to depress a button of the surgical navigation device 800 when the surgical navigation device 800 is disposed within the sterile cover 700. The soft touch button 718 can allow the user to power on or power off the surgical navigation device 800 when the surgical navigation device 800 is disposed within the sterile cover 700. The rear housing 716 can include a top surface 726. The top surface 726 can be flat. The top surface 726 can abut the ledge 608 of the transfer funnel 600.

The rear housing 716 can include one or more attachment grooves 728. The attachment groove 728 can be configured to receive the magnet 712. The rear housing 716 can include two attachment grooves 728. The housing 704 can include two magnets 712. The magnets 712 can be spaced apart. The magnets 712 can be parallel. The magnets 712 can couple to a bracket of a jig, as described herein. The front housing 708 can include a lumen 730. The transparent layer 710 can be positioned relative to the lumen 730. The transparent layer 710 can be coupled to the front housing 708. The transparent layer 710 can form a liquid resistant seal between the transparent layer 710 and the front housing 708. The transparent layer 710 can be adhered to the front housing 708, such as the use of an adhesive. The front housing 708 can include a top surface 732. The top surface 732 can be flat. The top surface 726 can abut the ledge 608 of the transfer funnel 600.

The transparent layer 710 is configured to overlie a touchscreen of the surgical navigation device 800. The transparent layer 700 can be compatible with the touchscreen of the surgical navigation device 800. In some embodiments, the touchscreen is a capacitive touchscreen. The transparent layer 700 can be configured to hold an electrical charge. The touchscreen can detect changes in the electric field when the conductive surface of the transparent layer 710 is contacted with a finger of the user. In some embodiments, the touchscreen is a resistive touchscreen. The touchscreen can respond to pressure when the user applies pressure to the transparent layer 710. The transparent layer 710 can be a thin layer. The transparent layer 710 can cover a portion of the surgical navigation device 800 when the surgical navigation device 800 is disposed within the sterile cover 700. The transparent layer 710 can cover a display of the surgical navigation device 800 when the surgical navigation device 800 is disposed within the sterile cover 700. The transparent layer 710 can allow the user to view images or instructions on the display of the surgical navigation device 800. The transparent layer 710 can allow the user to manipulate touch screen elements on the display of the surgical navigation device 800. The user can manipulate the touch screen by pressing against transparent layer 710 of the housing 704.

The sterile cover 700 can include a lid seal gasket 734. The lid 702 can include the lid seal gasket 734. The lid seal gasket 734 can fill the space between two or more mating surfaces. The lid seal gasket 734 can be disposed between the housing 704 and the lid 702 when the lid 702 is closed. The lid seal gasket 734 can form a liquid resistant seal between the housing 704 and the lid 702 when the lid 702 is closed. The lid seal gasket 734 can be disposed within a groove 736 of the lid 702. The top surface 732 of the front housing 708 and the top surface 726 of the rear housing 716 can compresses the lid seal gasket 734 when the lid 702 is closed. The lid seal gasket 734 can be compressed between the lid 702 and the housing 704 when the lid 702 is closed.

The lid 702 can include one or more alignment projections 738. The lid 702 can include any number of alignment projections 738 including one projection, two projections, three projections, four projections, five projections, six projections, seven projections, eight projections, ten projections, twelve projections, or any range of the foregoing values. The lid 702 can include one or more alignment projections 738 along one or more edges. The lid 702 can include one or more alignment projections 738 along one edge, two edges, three edges, four edges, or any range of two of the foregoing values. The lid 702 can include one or more alignment projections 738 along three edges. The lid 702 can include one or more alignment projections 738 along a first side edge. The lid 702 can include one or more alignment projections 738 along a second side edge, opposite the first side edge. The lid 702 can include one or more alignment projections 738 along an edge along the pivot axis. The alignment projections 738 can facilitate alignment of the surgical navigation device 800 within the sterile cover 700.

The lid 702 and the housing 704 can form a movable joint. The lid 702 and the housing 704 can form a hinge mechanism. The lid 702 and the housing 704 can allow for pivotal movement to open and close the lid 702 relative to the housing 704. The lid 702 and the housing 704 can allow for a limited range of movement such as 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, 180 degrees, or any range of two of the foregoing values. The lid 702 can rotate about the housing 704 approximately 90 degrees. The lid 702 can rotate about the housing 704 to open to be generally vertical. The lid 702 can rotate about the housing 704 to be perpendicular. The lid 702 can rotate about the housing 704 to be perpendicular with the top surfaces 223, 732 of the housing 704. The lid 702 can rotate about the housing 704 about a fixed axis of rotation. The lid 702 can rotate about one axis, while all other translations and rotations are prevented. The lid 702 and the housing 704 can have one degree of freedom.

The lid 702 can include two pins 740, as shown in FIG. 40B. The pins 740 can be aligned along the axis of rotation. The rear housing 716 can include two sockets 742. The sockets 742 can be aligned along the axis of rotation. The sockets 742 can receive the pins 740 to form a hinge joint. In other embodiments, the lid 702 can include sockets and the rear housing 716 can include pins. In other embodiments, the lid 702 and the housing 704 are joined via an axle. The lid 702 and the housing 704 can include barrels to receive the axle. Other pivot joints are contemplated.

The lid 702 and the housing 704 can include a mechanical fastener that joins the lid 702 and the housing 704 together when the lid 702 is closed. The lid 702 and the housing 704 can include the mechanical fastener that allows the lid 702 to be opened. The lid 702 and the housing 704 can form a latch. The lid 702 and the housing 704 can form a catch. The lid 702 and the housing 704 can form a snap fit. The lid 702 and the housing 704 can be held together in a specific position. The lid 702 and the housing 704 can easily close. The lid 702 and the housing 704 can require deliberate action to open the lid 702. The lid 702 and the housing 704 can form a locking mechanism. The lid 702 and the housing 704 can allow for reversable separation of the lid 702 and the housing 704.

The front housing 708 can include one or more projections 744. The front housing 708 can include one projection, two projections, three projections, four projections, or any range of two of the foregoing values. The front housing 708 can include two projections 744. The two projections 744 can be along the sides of the front housing 708. The two projections 744 can be diametrically opposed. The two projections 744 can be spaced apart. The lid 702 can include one or more grooves 746. The lid 702 can include one groove, two grooves, three grooves, four grooves, or any range of two of the foregoing values. The lid 702 can include two grooves 746. The two grooves 746 can be along the sides of the lid 702. The two grooves 746 can be diametrically opposed. The two grooves 746 can be spaced apart. The projection 744 can flex to interlock with the groove 746. The projection 744 can flex to release from the groove 746. The projections 744 can easily slide into the grooves 746 when the lid 702 is closed. The projections 744 can require a considerable force inward to disengage the grooves 746 to open the lid 702. The projections 744 can be biased outward. The user applies a force to compress the projections inward 744. In some embodiments, the lid 702 and the housing 704 can interlock along two sides of the housing 704. In some embodiments, the lid 702 and the housing 704 can interlock along more than one side.

The front housing 708 can include additional projections 745. The additional projections 745 can be along the front of the front housing 708. The lid 702 can include additional grooves 747. The additional projections 745 and the additional grooves 747 can provide additional interlocking features. The additional projections 745 and the additional grooves 747 can make opening the lid 702 more difficult by requiring additional force to disengage the lid 702. The additional projections 745 and the additional grooves 747 can be configured to prevent accidental opening of the lid 702. In some embodiments, the lid 702 and the housing 704 can interlock along three sides of the housing 704. In some embodiments, the lid 702 and the housing 704 can interlock along more than two sides.

The projection 744 can require significant force to disengage from the groove 746. The projection 744 and the groove 746 can be configured to be releasable upon specific directed force on the projection 744. The lid 702 can require significant force to disengage from the housing 704. The lid 702 can be opened upon specific directed force on the projection 744. The lid 702 can be opened upon inward force on two diametrically opposed projections 744. The lid 702 can be opened upon a deliberate coordination of significant forces.

The housing 704 can form an upper surface. The rear housing 716 can include the top surface 726. The top surface 726 can be flat. The front housing 708 can include a top surface 732. The top surface 732 can be flat. The top surfaces 726, 732 of the housing 704 can be form a continuous flat surface. The top surfaces 726, 732 of the housing 704 can support the ledge 608 of the transfer funnel 600.

In some embodiments, the sterile cover 700 can be disposable. The sterile cover 700 can be single use. The sterile cover 700 can be manufactured and packaged in a sterile state. The sterile cover 700 can provide a sterile barrier between the reusable surgical navigation device 800 and the surgical field. Once the sterile cover 700 has been used during a surgical procedure, the sterile cover 700 can be discarded and the surgical navigation device 800 can be reused. The sterile cover 700 can be a single use sterile barrier separating the surgical navigation device 800 from the sterile field. The sterile cover 700 can be provided to the user with instructions for single use.

In some embodiments, the sterile cover 700 can be reusable. The sterile cover 700 can be configured for use with multiple procedures. The sterile cover 700 can be sterilized after use. The sterile cover 700 can be sterilized using any method described herein. The sterile cover 700 can be autoclaved. The sterile cover 700 can be provided to the user with instructions for sterilization.

The sterile cover 700 can receive the surgical navigation device 800. The sterile cover 700 can include the opening 706. The housing 704 can receive the surgical navigation device 800 when the lid 702 is open. In some methods, the transfer funnel 600 can be utilized to facilitate insertion of the surgical navigation device 800 into the sterile cover 700. In some methods, the transfer funnel 600 is not utilized. The surgical navigation device 800 can be received within the sterile cover 700 without the surgical navigation device 800 contacting any outside surface of the sterile cover 700, thereby maintaining the sterility.

The housing 704 can include one or more alignment features 750. The alignment features 750 can be projections and/or grooves. The alignment features 750 can be positioned along one or more interior walls of the housing 704. The rear housing 716 can include one or more alignment features 750. The alignment features 750 can include longitudinally oriented projections. The alignment features 750 can extend the entire length of the housing 704. The alignment features 750 can extend a portion of the length of the housing 704. The alignment features 750 can be tapered. The alignment features 750 can be integrally formed with the rear housing 716. The front housing 708 can include one or more alignment features 750. The alignment features 750 can include horizontally oriented projections. The alignment features 750 can extend the entire width of the housing 704. The alignment features 750 can extend a portion of the width of the housing 704. The alignment features 750 can be integrally formed with the front housing 708. The alignment features 750 can facilitate the positioning of the surgical navigation device 800 within the housing 704. The alignment features 750 can reduce gaps between the housing 704 and the surgical navigation device 800. The alignment features 750 can allow easy, low precision insertion of the surgical navigation device 800 into the housing 704.

The housing 704 can include one or more angled ramps 752. The angled ramps 752 can be positioned along one or more interior walls of the housing 704. The rear housing 716 can include the angled ramps 752. The angled ramps 752 can be integrally formed with a portion of the housing 704. The angled ramps 752 can be integrally formed with the rear housing 716. The angled ramps 752 can extend from the alignment features 750. The angled ramps 752 can facilitate positioning of the surgical navigation device 800 within the housing 704. The angled ramps 752 can ensure that the surgical navigation device 800 is positioned against toward the transparent layer 710.

The housing 704 can receive the surgical navigation device 800 in a single orientation. The housing 704 can receive the surgical navigation device 800 in a single position. The housing 704 can receive the surgical navigation device 800 in a single rotation. The housing 704 can receive the surgical navigation device 800 with a known orientation of the inertial sensors. The housing 704 can receive the surgical navigation device 800 with a known orientation for sensor accuracy. The housing 704 can receive the surgical navigation device 800 to limit or prevent any movement therebetween. The housing 704 can receive the surgical navigation device 800 to limit or prevent any forward and backward movement. The housing 704 can receive the surgical navigation device 800 to limit or prevent side-to-side movement. The housing 704 can receive the surgical navigation device 800 wherein the only movement is insertion and removal of the surgical navigation device 800 from the housing 704. The housing 704 can receive the surgical navigation device 800 with minimal tolerances. The housing 704 can receive the surgical navigation device 800 with minimal gaps. The housing 704 can receive the surgical navigation device 800 with internal surfaces of the sterile cover 700 contacting one or more sides of the surgical navigation device 800. The housing 704 can receive the surgical navigation device 800 with internal surfaces of the sterile cover 700 contacting a front and back of the surgical navigation device 800. The housing 704 can receive the surgical navigation device 800 with internal surfaces of the sterile cover 700 contacting sides of the surgical navigation device 800. The housing 704 can receive the surgical navigation device 800 in a single orientation.

The sterile cover 700 can allow attachment to a jig. The sterile cover 700 can include one or more magnets 712. The magnets 712 can couple to a bracket of the jig. The bracket of the jig can include compatible magnets.

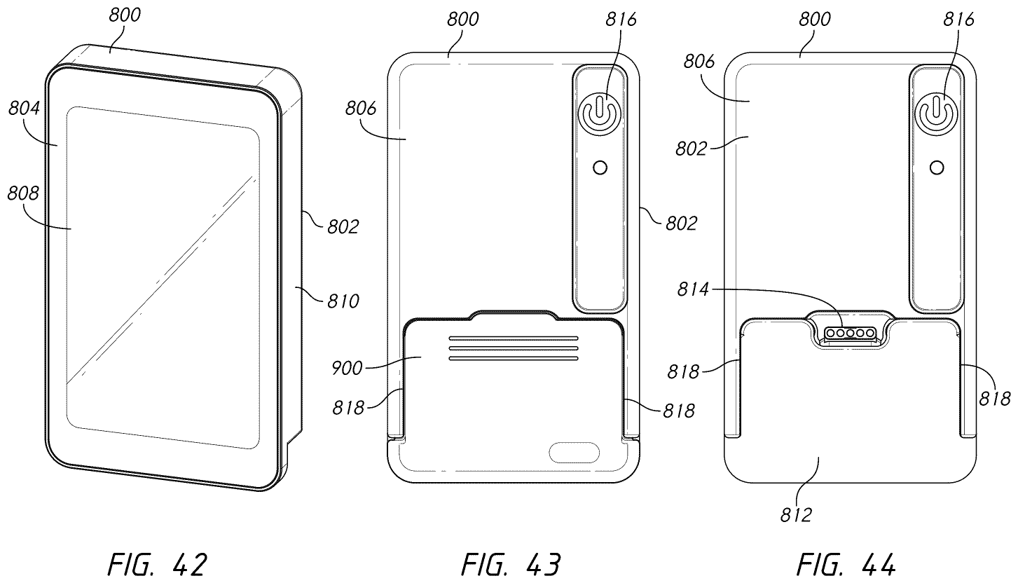

FIG. 42 is front perspective view of the surgical navigation device 800. FIG. 43 is back view of the surgical navigation device 800. FIG. 44 is another back view of the surgical navigation device 800. The surgical navigation device 800 can have any feature of the surgical navigation device 300 described herein.

The surgical navigation device 800 can comprise a compact, generally hand-held, portable device for use in the joint replacement procedure. The surgical navigation device 800 can comprise a generally rectangular-shaped, box-like structure having an outer housing 802. The surgical navigation device 800 can include a front 804 and a back 806. The surgical navigation device 800 can comprise sides 810. The surgical navigation device 800 can form an enclosure to house internal components such as inertial sensors.

The front 804, or a portion of the front 804, of the surgical navigation device 800 can comprise a display 808. In some embodiments, the display 808 can be a separate component from the outer housing 802. In some embodiments, the display 808 can be integrated on or within the outer housing 802. The display 808 can be include flat-panel display technology. The display 808 can include a liquid crystal display (“LCD”). The display 808 can include light-emitting diodes (“LED”) as the pixels to create images or text. The display 808 can be sufficiently sized to allow the user to view numbers, lettering, and/or symbols on the display 808 while performing the surgical procedure. The display 808 can be a touchscreen. The user can provide input by touching the display 808. The user can provide input by touching the transparent layer 710 that overlies the display 808 when the surgical navigation device 800 is received within the sterile cover 700. The surgical navigation device 800 can comprise a plurality of touchscreen buttons. The buttons can be activated, for example, by a finger of the user. The display 808 can be viewed and manipulated by the user during the surgical procedure.

FIGS. 43 and 44 show the back 806 of the surgical navigation device 800. The back 806 can include an attachment groove 812. The surgical navigation device 800 can receive the battery pack 900. The battery pack 900 can fit within the attachment groove 812 of the surgical navigation device 800. The back 806 can include an electrical interface 814. The battery pack 900 can include a complementary electrical interface configured to couple with the electrical interface 814. The back 806 can include a power button 816. The power button 816 can be offset to one side of the surgical navigation device 800. The power button 816 can project from the back 806 of the surgical navigation device 800.

The surgical navigation device 800 can comprise an electrical system. The electrical system can include an electronic control unit that communicates with one or more sensors and the display 808, among other features. The electronic control unit can receive input from the one or more sensors and transmits output to the display 808. The electronic control unit can be configured to receive and send data, as well as perform calculations based on data. The electronic control unit can include one or more processors, program logic, or other substrate configurations representing data and instructions. The surgical navigation device 800 can have any feature of the surgical navigation device 300 described herein. The orientation or position of the surgical navigation device 800 can be used to provide feedback to a user during the surgical procedure, such as a joint replacement surgery.

The surgical navigation device 800 can include one or more sensors. The one or more sensors can include a gyroscopic sensor, an accelerometer sensor, a tilt sensor, a magnetometer, and/or one or more sensors and/or other similar device or devices configured to measure, and/or facilitate determination of, an orientation of the surgical navigation device 800. The one or more sensors can be configured to provide measurements relative to a reference point(s), line(s), plane(s), and/or gravitational zero. The one or more sensors can be inertial sensors. The surgical navigation device 800 can comprise one or more circuit boards and/or other circuitry. The calculations can be performed by software executed by the electronic control unit. In some embodiments, the electronic control unit can perform a protocol to assess the reliability of the calculated measurements.

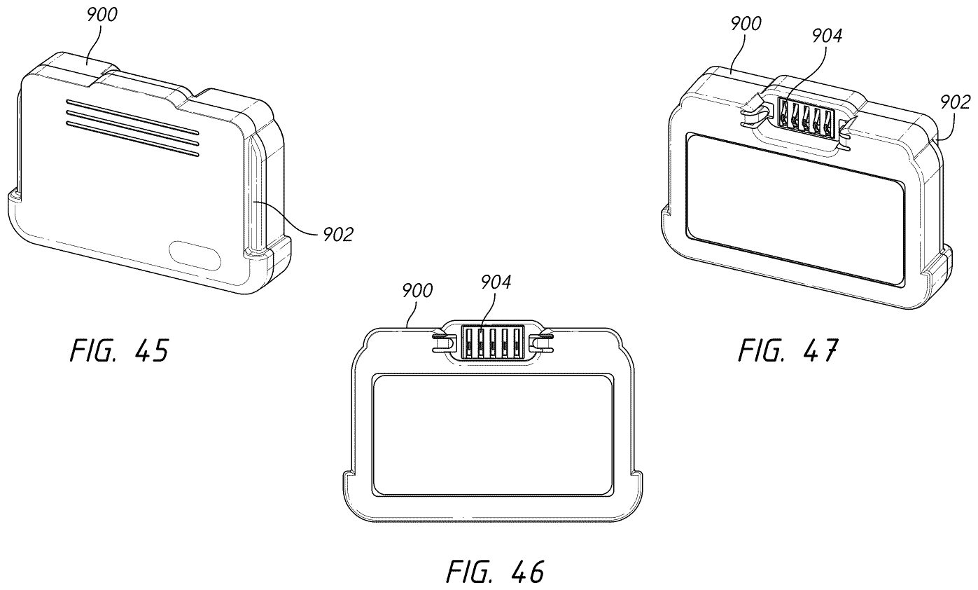

FIG. 45 is front perspective view of the battery pack 900. FIG. 46 is back view of the battery pack 900. FIG. 47 is back perspective view of the battery pack 900. The battery pack 900 can be single use. The battery pack 900 can be disposable. The battery pack 900 can be for power delivery. The battery pack 900 can be for authentication security. The battery pack 900 can be for license management.

The battery pack 900 can include one or more power sources configured to supply power to the electrical components of the surgical navigation device 800. The battery pack 900 can include one or more batteries. The battery pack 900 can include any feature of the battery pack 400 described herein. The battery pack 900 can include one or more processors for processing data and instructions. The battery pack 900 can supply power to the one or more sensors. The battery pack 900 can comprise one or more circuit boards and/or other circuitry.

The surgical navigation device 800 can communicate with the battery pack 900 . . . . The battery pack 900 can communicate with a processor on the surgical navigation device 800 for authentication. The authentication result is communicated to the processor on the surgical navigation device 800. In some embodiments, the software of the surgical navigation device 800 and/or the battery pack 900 can provide authentication. In some embodiments, the software of the surgical navigation device 800 and/or the battery pack 900 can provide licensing.

The surgical navigation device 800 can be reusable. The surgical navigation device 800 can be non-sterile. The surgical navigation device 800 and the battery pack 900 can be provided as separate components. The surgical navigation device 800 can have the number of uses limited by a programmed usage counter. The battery pack 900 can be single use. The battery pack 900 can include a usage counter to ensure that the battery pack 900 is only used once. The battery pack 900 can provide authentication and license management. The battery pack 900 can be licensed for use only with a single type of surgical procedures.

The battery pack 900 can include grooves 902 that can extend partially, or entirely, along the sides of the battery pack 900. The projections 818 of the surgical navigation device 800 can be received by the corresponding grooves 902 from the battery pack 900, so as to couple the battery pack 900 to the surgical navigation device 800. The battery pack 900 can slide upward into engagement with the attachment groove 812 of surgical navigation device 800.

The battery pack 900 can include a complementary electrical interface 904. The complementary electrical interface 904 is configured to couple with the electrical interface 814 of the surgical navigation device 800. The battery pack 900 can provide electrical power through the electrical interface 404.

The battery pack 900 can be used for authentication. The battery pack 900 and the surgical navigation device 800 can mate via a physical connection. The battery pack 900 can provide authentication through any method described herein.

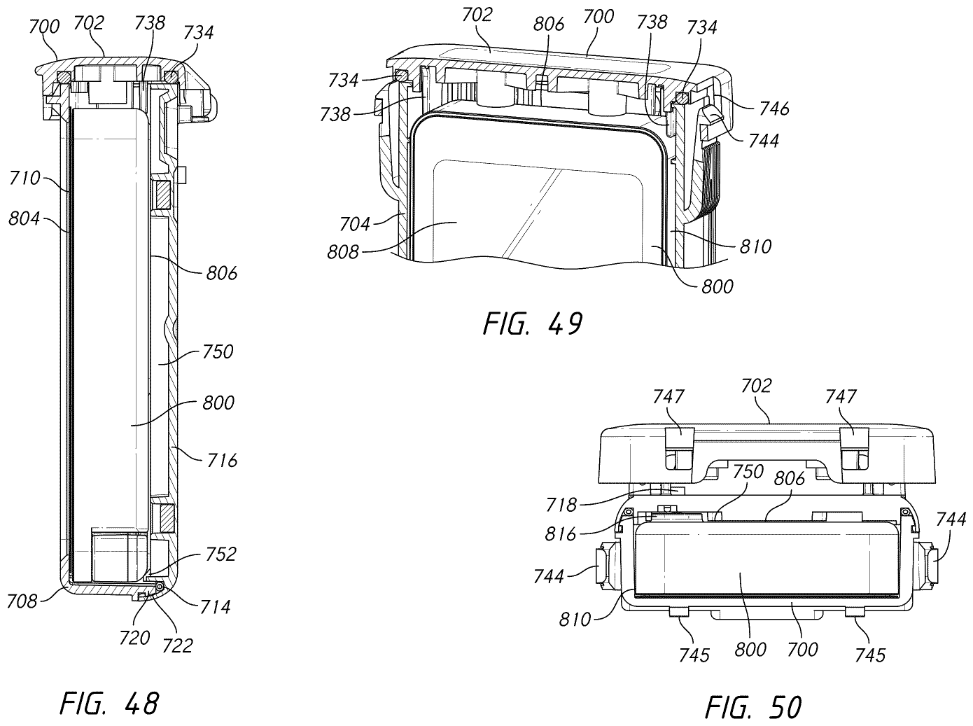

FIG. 48 is a cross-sectional view of the sterile cover 700 and the surgical navigation device 800 with the lid 702 closed. FIG. 49 is another cross-sectional view of the sterile cover 700 and the surgical navigation device 800 with the lid 702 closed. FIG. 50 is a top view of the sterile cover 700 and the surgical navigation device 800 with the lid 702 opened.

The front housing 708 and the rear housing 716 can be coupled during manufacturing. The front housing 708 can include the interlocking projection 720. The rear housing 716 can include the interlocking groove 722. The housing seal gasket 714 can be disposed between the front housing 708 and the rear housing 716. The front housing 708 and the rear housing 716 can be provided to the user as permanently coupled. The housing seal gasket 714 can form a liquid resistant seal.

The lid 702 and the housing 704 can be provided to the user as permanently coupled. The lid 702 can open and close relative to the housing 704. The sterile cover 700 can be provided to the user with the lid 702 coupled to the housing 704. The rear housing 716 can include two sockets 742. The sockets 742 of the rear housing 716 can receive the pins 740 of the lid 702 to form a hinge joint. The lid seal gasket 734 can be disposed within the lid 702. The lid 702 and the lid seal gasket 734 can be provided to the user as permanently coupled. The lid 702 compresses the lid seal gasket 734 against the top surface 732 of the front housing 708 and the top surface 726 of the rear housing 716 when the lid 702 is closed. The lid seal gasket 734 can form a liquid resistant seal. The housing seal gasket 714 and the lid seal gasket 734 can limit or prevent the escape of liquid from the sterile cover 700.

The lid 702 can be closed. The front housing 708 can include two projections 744. The lid 702 can include two grooves 746. The projections 744 and the grooves 746 can interlock. The projections 744 can be biased outward to engage the grooves 746. The user can manually push the projections 744 inward to release the projections 744 from the grooves 746. The user can manually push two projections 744 inward to release the lid 702. In some embodiments, the user must push two projections 744 which are diametrically opposed. In some embodiments, the projections 744 are designed to require significant targeted force to release the projections 744 from the grooves 746. The lid 702 and the housing 704 are configured to limit inadvertent opening of the lid 702 during use. For instance, the projections 744 may require significant force to release the projections 744 from the grooves 746. For instance, the projections 744 may require significant force to release the lid 702. For instance, two or more projections 744 may need to be actuated to release the lid 702. The plurality of projections 744 may limit inadvertent disassembly of the lid 702 during use. The position of projections 744 may limit inadvertent disassembly of the lid 702 during use. The strength of the connection between the grooves 746 from the projections 744 may limit inadvertent disassembly of the lid 702 during use.

The alignment projections 738 of the lid 702 can facilitate alignment of the surgical navigation device 800 within the housing 704. The alignment projections 738 of the lid 702 can push down on the surgical navigation device 800 within the housing 704. The alignment projections 738 of the lid 702 can push the surgical navigation device 800 forward against the transparent layer 710. The alignment projections 738 of the lid 702 can allow the lid 702 to close only if the surgical navigation device 800 is received in a single orientation. The alignment projections 738 of the lid 702 can provide a downward force on the surgical navigation device 800. The alignment projections 738 of the lid 702 can provide a frontward force on the surgical navigation device 800. The alignment projections 738 can fill gaps between the surgical navigation device 800 and the sterile cover 700. The alignment projections 738 of the lid 702 can allow the lid 702 to close if the surgical navigation device 800 is correctly positioned within the sterile cover. In some embodiments, the lid 702 only closes if the surgical navigation device 800 is in a single orientation.

The housing 704 can include one or more alignment features 750. The alignment features 750 can include longitudinally oriented projections. The alignment features 750 can include horizontally oriented projections. The alignment features 750 can position the surgical navigation device 800 forward. The alignment features 750 can position the surgical navigation device 800 toward transparent layer 710. The alignment features 750 can contact the surgical navigation device 800. The alignment features 750 can contact the surgical navigation device 800 with minimal tolerances. The housing 704 can include one or more angled ramps 752. The angled ramps 752 can push the surgical navigation device 800 toward the transparent layer 710 of the sterile cover 700. The rear housing 716 can include the angled ramps 752. The angled ramps 752 of the rear housing 716 can provide a frontward force on the surgical navigation device 800.

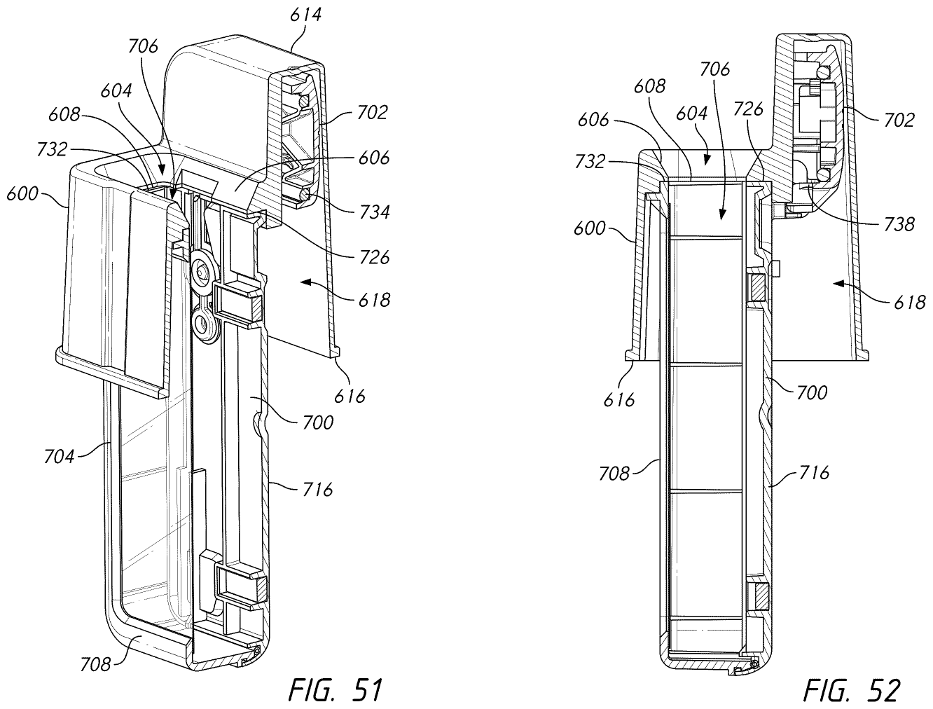

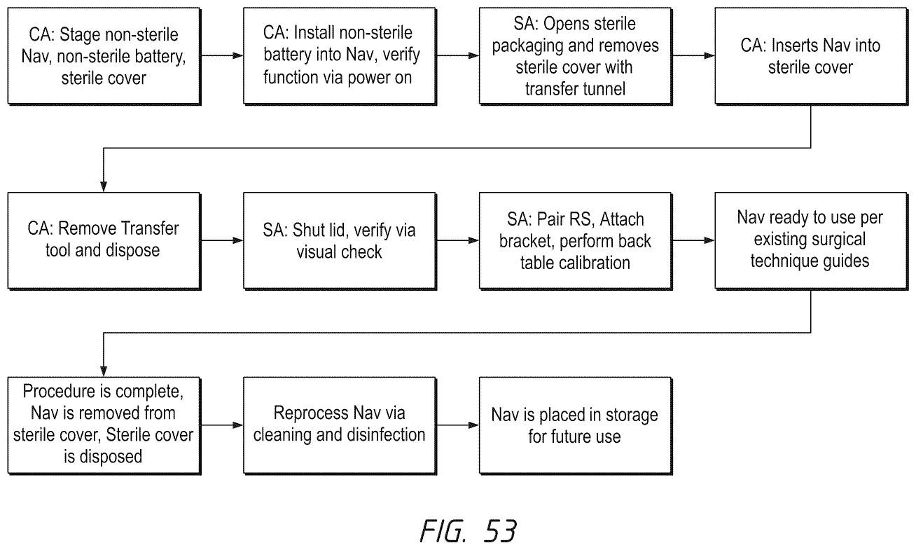

FIG. 51 is a cross-sectional view of the transfer funnel of 600 and the sterile cover 700. FIG. 52 is another cross-sectional view of the transfer funnel 600 and the sterile cover 700. The transfer funnel 600 can limit or prevent contact between sterile and non-sterile components during aseptic transfer. The transfer funnel 600 can be sterile and the sterile cover 700 can be sterile. The transfer funnel 600 can cover a significant portion of the sterile cover 700 during use. The transfer funnel 600 can enclose an upper portion of the sterile cover 700 during use.

The transfer funnel 600 can be positioned over the sterile cover 700. The transfer funnel 600 can include the ledge 608. The ledge 608 can function as a stop against the sterile cover 700. The ledge 608 can function as a stop against the housing 704. The ledge 608 can rest against the top surface of the housing 704. The ledge 608 can abut the top surface 726 of the rear housing 716 and abut the top surface 732 of the front housing 708. The ledge 608 can form a flat surface for abutting top surface of the housing 704.

In some embodiments, the transfer funnel 600 does not extend into the housing 704. The transfer funnel 600 can be entirely external to the housing 704. The transfer funnel 600 can surround at least a portion of the sterile cover 700. The transfer funnel 600 does not enter the sterile cover 700. In some embodiments, the transfer funnel 600 does not extend into the opening 706 of the housing 704. The transfer funnel 600 can abut the top surfaces 726, 732 of the housing 704. The transfer funnel 600 does not extend into any portion of the sterile cover 700. The transfer funnel 600 can be entirely external to the sterile cover 700. The transfer funnel 600 does not enter the sterile cover 700 at any point during aseptic transfer. The transfer funnel 600 can be supported by the top surfaces 726, 732 of the housing 704. The transfer funnel 600 can be supported by the opened lid 702. The transfer funnel 600 can extend along the perimeter of the sterile cover 700. The transfer funnel 600 can extend over the sterile cover 700 when viewed from the top. The transfer funnel 600 can extend downward from the top of the sterile cover 700. The transfer funnel 600 can encircle the sterile cover 700 along a portion of the length of the sterile cover 700. The transfer funnel 600 can be placed on top of the sterile cover 700. The transfer funnel 600 can extend over a surface area of the sterile cover 700. The transfer funnel 600 can extend over the top surface of the sterile cover 700 when the lid 702 is opened.

The transfer funnel 600 can include the lumen 604. The sterile cover 700 can include the opening 706. The lumen 604 of the transfer funnel 600 and the opening 706 of the sterile cover 700 can be the same shape. The lumen 604 of the transfer funnel 600 and the opening 706 of the sterile cover 700 can be the same size. The lumen 604 of the transfer funnel 600 and the opening 706 of the sterile cover 700 can be coaxial. The lumen 604 of the transfer funnel 600 and the opening 706 of the sterile cover 700 can be aligned. The transfer funnel 600 can be supported at multiple points of engagement with the sterile cover 700. The transfer funnel 600 can be supported by the top surfaces 726, 732 of the housing 704. The transfer funnel 600 can be supported by the opened lid 702. The transfer funnel 600 can be supported by the front housing 708. The transfer funnel 600 can be supported by the rear housing 716. The transfer funnel 600 can extend over the sterile cover 700 when the lid 702 is open. The cavity of the transfer funnel 600 can receive the upper portion of the sterile cover 700. The extension 614 can accommodate the lid 702 when the lid 702 is open. The extension 614 can be shaped to receive the lid 702 which can be perpendicular when opened. The extension 614 can extend the entire length of the lid 702, or a portion thereof. The body 602 can extend the entire length of the lid 702. The body 602 can extend below the lid 702. The body 602 can extend approximately half the length of the sterile cover 700.

The transfer funnel 600 can cover the lid 702 when the ledge 608 of the transfer funnel 600 abuts the top surfaces 726, 732 of the housing 704. The lower edge 616 of the transfer funnel 600 can extend below the lid 702. The cavity 618 can receive the lid 702. The cavity 618 can be shaped to receive the lid 702 when the lid 702 is opened. The cavity 618 can surround the lid 702. The cavity 618 can surround the upper portion of the sterile cover 700. The opened lid 702 can stabilize the transfer funnel 600 when the transfer funnel 600 receives the sterile cover 700. The lid 702 can abut an internal surface of the transfer funnel 600. The transfer funnel 600 can be supported by the opened lid 702. The transfer funnel 600 can be lowered relative to the sterile cover 700. The transfer funnel 600 can be raised to remove the transfer funnel 600. The transfer funnel 600 can have a single orientation relative to the sterile cover 700 during use.

The ledge 608 of the transfer funnel 600 can rest against the top surface 726, 732 of the housing 704. The ledge 608 of the transfer funnel 600 can abut the housing 704 of the sterile cover 700. The transfer funnel 600 does not extend into the opening 706 of the housing 704. The transfer funnel 600 can be external to the housing 704. The opening 706 of the housing 704 can align with the lumen 604 of the transfer funnel 600. The cavity 618 of the transfer funnel 600 can receive the lid 702. The lid 702 can be within the transfer funnel 600 when the transfer funnel 600 is positioned relative to the sterile cover 700. In some embodiments, the transfer funnel 600 extends below the lid 702. The transfer funnel 600 can prevent accidental contact between the surgical navigation device 800 and the lid 702 when the transfer funnel 600 is positioned. The transfer funnel 600 can prevent accidental contact between the surgical navigation device 800 and the housing 704 when the transfer funnel 600 is positioned. The transfer funnel 600 can prevent accidental contact between the surgical navigation device 800 and the sterile cover 700 when the transfer funnel 600 is positioned. The transfer funnel 600 provides a sterile barrier between the surgical navigation device 800 and the sterile cover 700 during insertion of the surgical navigation device 800 into the housing 704.

FIG. 53 is a method of use. The transfer funnel 600 can be used in any method of use described herein. The sterile cover 700 can be used in any method of use described herein. The surgical navigation device 800 can be used in any method of use described herein. The battery pack 900 can be used in any method of use described herein. The steps can be performed by the circulating, non-sterile assistant (CA). The steps can be performed by the sterile assistant (SA).

The method can include staging one or more components. The method can include staging the non-sterile surgical navigation device 800. The method can include staging the non-sterile battery pack 900. The method can include staging the sterile cover 700. In some embodiments, the sterile cover 700 and the transfer funnel 600 are together in a single sterile package. In some embodiments, the sterile cover 700 and the transfer funnel 600 are assembled in the sterile packaging. In some embodiments, the transfer funnel 600 and the sterile cover 700 are within a single use kit. In some embodiments, the sterile cover 700, the transfer funnel 600, and the battery pack 900 are within a single use kit. The staging can be performed by the circulating, non-sterile assistant.

The method can include installing the battery pack 900 into the surgical navigation device 800. The method can include verifying functioning of the battery pack 900. The method can include verifying functioning of the surgical navigation device 800. The method can include verifying functioning by powering on the surgical navigation device 800. Referring to FIGS. 42-47, the battery pack 900 can slide within the attachment groove 812 of the surgical navigation device 800. The surgical navigation device 800 and the battery pack 900 can be coupled. The electrical interface 904 of the battery pack 900 can couple with the electrical interface 814 of the surgical navigation device 800. The battery pack 900 can provide licensing and authentication in some methods. The installing of the battery pack 900 can be performed by the circulating, non-sterile assistant

The method can include opening sterile packaging. The method can include opening the sterile packaging of the sterile cover 700. The method can include opening the sterile packaging of the transfer funnel 600. In some embodiments, the sterile cover 700 and the transfer funnel 600 can be packaged together in a single use kit. The method can include opening the sterile packaging of the sterile cover 700 with the transfer funnel 600. The method can include removing the sterile cover 700 with the transfer funnel 600 from the sterile packaging. The lid 702 of the sterile cover 700 can be open. The transfer funnel 600 can be disposed over the opened lid 702 as shown in FIGS. 51 and 52. In some methods, the user does not need to assemble the transfer funnel 600 and the sterile cover 700. In some methods, the transfer funnel and the sterile cover 700 are pre-assembled. In some embodiments, the sterile cover 700 and the transfer funnel 600 are sterile, single use components. The opening of the sterile packaging can be performed by the sterile assistant.

The method can include inserting the surgical navigation device 800 into the sterile cover 700. During insertion, the transfer funnel 600 covers an upper portion of the sterile cover 700. The transfer funnel 600 provides a temporary sterile barrier between the surgical navigation device 800 which is non-sterile and the sterile cover 700 which is sterile. The method can include inserting the surgical navigation device 800 through the transfer funnel 600. The transfer funnel 600 can include a lumen 604 to receive the surgical navigation device 800. The method can include inserting the surgical navigation device 800 into the housing 704. The housing 704 can include the opening 706 to receive the surgical navigation device 800.

The circulating, non-sterile assistant can touch non-sterile components during aseptic transfer. The circulating, non-sterile assistant can touch the surgical navigation device 800. The inserting of the surgical navigation device 800 can be performed by the circulating, non-sterile assistant. The circulating, non-sterile assistant can insert the surgical navigation device 800 into the transfer funnel 600 and the sterile cover 700. In some methods, the circulating, non-sterile assistant can touch only the surgical navigation device 800 during insertion of the surgical navigation device 800. In some methods, the circulating, non-sterile assistant does not touch the sterile cover 700 during insertion of the surgical navigation device 800. In some methods, the circulating, non-sterile assistant does not touch the transfer funnel 600 during insertion of the surgical navigation device 800. In some methods, the circulating, non-sterile assistant only touches the surgical navigation device 800.

The sterile assistant can touch sterile components during aseptic transfer. The sterile assistant can touch the sterile cover 700. The sterile assistant can touch the transfer funnel 600, in some methods. The sterile assistant does not touch the surgical navigation device 800 during insertion of the surgical navigation device 800. In some methods, the sterile assistant can touch the lower portion of the sterile cover 700. The transfer funnel 600 can at least partially cover the hand of the sterile assistant during aseptic transfer. The sterile assistant can hold the sterile cover 700 below the transfer funnel 600 during insertion of the surgical navigation device 800. In some methods, the sterile assistant does not touch the transfer funnel 600 during insertion of the surgical navigation device 800. In some methods, the sterile assistant only touches the sterile cover 700. In some methods, the sterile assistant does touch the transfer funnel 600 during insertion of the surgical navigation device 800. The sterile assistant can hold the sterile cover 700 and the transfer funnel 600 at the same time. In some methods, the sterile assistant and the circulating, non-sterile assistant do not touch the transfer funnel 600 during insertion of the surgical navigation device 800.

The method can include lifting the transfer funnel 600 relative to the sterile cover 700. The method can include discarding the transfer funnel 600. The removing and disposing of the transfer funnel 600 can be performed by the circulating, non-sterile assistant.

The method can include closing the lid 702. The method can include verifying that the lid 202 is closed via a visual check. The closing of the lid 702 can be performed by the sterile assistant.

In some methods, the method can include pairing the surgical navigation device 800 and a second surgical navigation device 800. The second surgical navigation device 800 can have any feature of the surgical navigation device 300, 800 described herein. The second surgical navigation device 800 can have fewer features than the surgical navigation device 300, 800 described herein. In some embodiments, the second surgical navigation device 800 does not include a display. In some embodiments, the surgical navigation device 800 can have any feature of the orientation device 172 described in U.S. application Ser. No. 13/800,620 filed Mar. 13, 2013 and/or U.S. application Ser. No. 15/550,564 filed Aug. 11, 2014, both of which are incorporated by reference herein in their entirety. In some embodiments, the second surgical navigation device 800 can have any feature of the orientation device 204 described in U.S. application Ser. No. 13/800,620 filed Mar. 13, 2013 and/or U.S. application Ser. No. 15/550,564 filed Aug. 11, 2014, both of which are incorporated by reference herein in their entirety. The pairing can include communication of data between the surgical navigation device 800 and the second surgical navigation device 800. In some methods, only one surgical navigation device 800 is utilized. In some methods, two surgical navigation devices 800 are utilized. In some methods, three surgical navigation devices 800 are utilized. The pairing can be performed by the sterile assistant.

The method can include attaching the bracket. The bracket can attach via magnets to the sterile cover 700. The bracket can be a portion of a jig used for a joint replacement procedure. The attaching of the bracket can be performed by the sterile assistant. The method can include performing back table calibration of the surgical navigation device 800. The calibration can be performed by the sterile assistant.

The method can include surgical navigation. The surgical navigation device 800 can be ready to use per surgical technique guides. The surgical navigation device 800 can be used in a joint replacement procedure. The surgical navigation device 800 can be used for knee replacement methods. The surgical navigation device 800 can be used for hip replacement methods. The surgical navigation device 800 can remain within the sterile cover 700 during surgical navigation. The surgical navigation device 800 can be manipulated while within the sterile cover 700. The user can view instructions through the transparent layer 710. The user can view measurements through the transparent layer 710. The user can interact with the surgical navigation device 800 by pressing against the transparent layer 710. The user can interact with the surgical navigation device 800 by manipulating the touchscreen.

The method can include completing the procedure. The method can include removing the surgical navigation device 800 from the sterile cover 700. The method can include disposing the sterile cover 700. The sterile cover 700 can be single use. In some methods, the method can include removing the battery pack 900. The method can include disposing of the battery pack 900. The battery pack 900 can be single use.

The method can include reprocessing the surgical navigation device 800. The method can include cleaning the surgical navigation device 800. The method can include disinfecting the surgical navigation device 800. The surgical navigation device 800 can be reusable. The surgical navigation device 800 can be non-sterile.

The method can include storing the surgical navigation device 800. The surgical navigation device 800 can be placed in storage for another surgical procedure. The method can include repeating one or more steps. The method can include repeating one or more steps with a second sterile cover 700. The method can include repeating one or more steps with a second transfer funnel 600. The method can include repeating one or more steps with a second battery pack 900. The method can include repeating one or more steps while reusing the surgical navigation device 800.

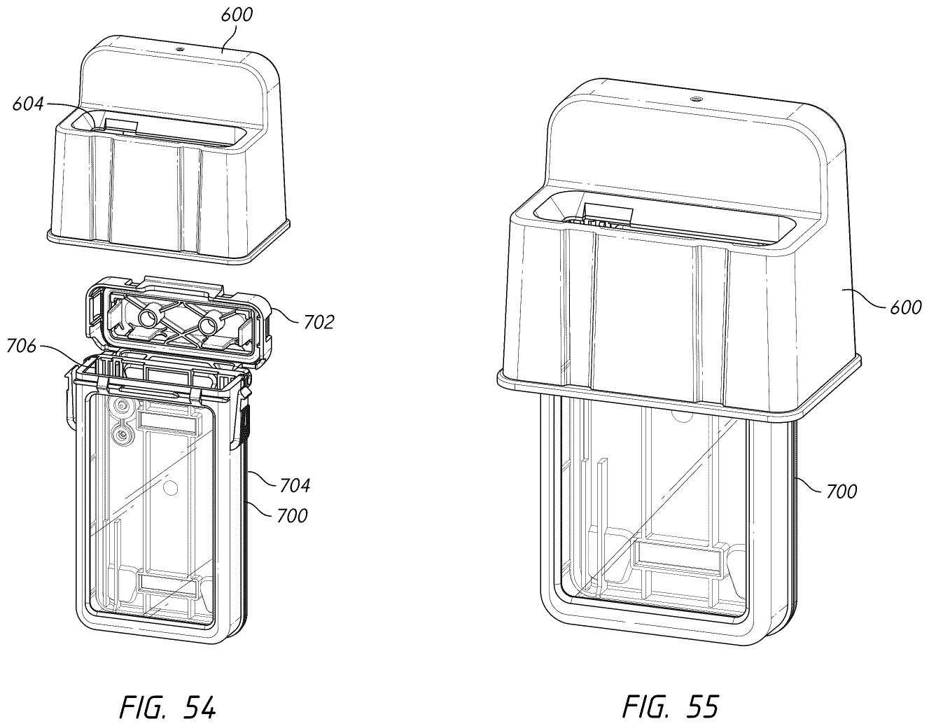

FIG. 54 illustrates the transfer funnel 600 and the sterile cover 700 in an exploded view. FIG. 55 illustrates the transfer funnel 600 and the sterile cover 700 when assembled. In some methods, the transfer funnel 600 and the sterile cover 700 can be pre-assembled for use. In some methods, the transfer funnel 600 and the sterile cover 700 can be pre-assembled in the sterile packaging. In some methods, the transfer funnel 600 and the sterile cover 700 can be assembled during manufacturing. The transfer funnel 600 can be lowered relative to the sterile cover 700. The transfer funnel 600 can abut one or more external surface of the sterile cover 700. The transfer funnel 600 can abut the top surface 726, 732 of the housing 704. The transfer funnel 600 can abut the opened lid 702. The transfer funnel 600 can receive the upper portion of the sterile cover 700 within the cavity 618 of the transfer funnel 600. The transfer funnel 600 can include the lumen 604 and the sterile cover 700 can include the opening 706. The lumen 604 and the opening 706 can be aligned when the transfer funnel 600 and the sterile cover 700 are assembled. The transfer funnel 600 can include the extension 614. The extension 614 can visually align with the opened lid 702. The assembly of the transfer funnel 600 and the sterile cover 700 can be intuitive.

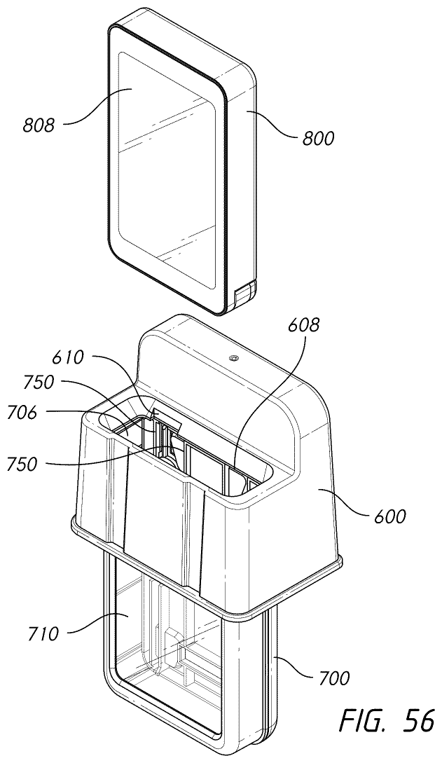

FIG. 56 is a method of use of the transfer funnel 600, the sterile cover 700, and the surgical navigation device 800. The transfer funnel 600 can include the alignment slot 610. The alignment slot 610 can accommodate the power button 816 of the surgical navigation device 800, shown in FIG. 43, as the surgical navigation device 800 is inserted into the transfer funnel 600. During insertion, the power button 816 of the surgical navigation device 800 can slide relative to the alignment slot 610. The alignment slot 610 can correctly orient the surgical navigation device 800 during insertion into the transfer funnel 600. The surgical navigation device 800 can be inserted into the transfer funnel 600 in a single orientation. The surgical navigation device 800 can be oriented such that the display 808 faces toward the transparent layer 710.

The housing 704 can include one or more alignment features 750. The alignment features 750 can be one or more projections positioned along one or more interior walls of the housing 704. The alignment features 750 can accommodate the power button 816 of the surgical navigation device 800 as the surgical navigation device 800 is inserted into the sterile cover 700. During insertion, the power button 816 of the surgical navigation device 800 can slide relative to the alignment features 750. The alignment features 750 can correctly orient the surgical navigation device 800 during insertion into the sterile cover 700. The surgical navigation device 800 can be inserted into the sterile cover 700 in a single orientation.

The battery pack 900 can be installed with the surgical navigation device 800 before inserting the surgical navigation device 800 into the transfer funnel 600 and the sterile cover 700. The battery pack 900 can provide power. The battery pack 900 can provide authorization. The battery pack 900 can provide license management. The battery pack 900 and the surgical navigation device 800 can use a Keyed-Hash Message Authentication Code (HMAC) to verify authenticity of the battery pack 900. The battery pack 900 can include a counter to ensure that the battery pack 900 can only be used for one surgical procedure. The battery pack 900 can include memory that stores the type of license. The license can relate to the joint of the joint procedure. In some methods, the surgical navigation device 800 is powered on before inserting the surgical navigation device 800 into the sterile cover 700. In some methods, the surgical navigation device 800 is powered on after inserting the surgical navigation device 800 into the sterile cover 700.

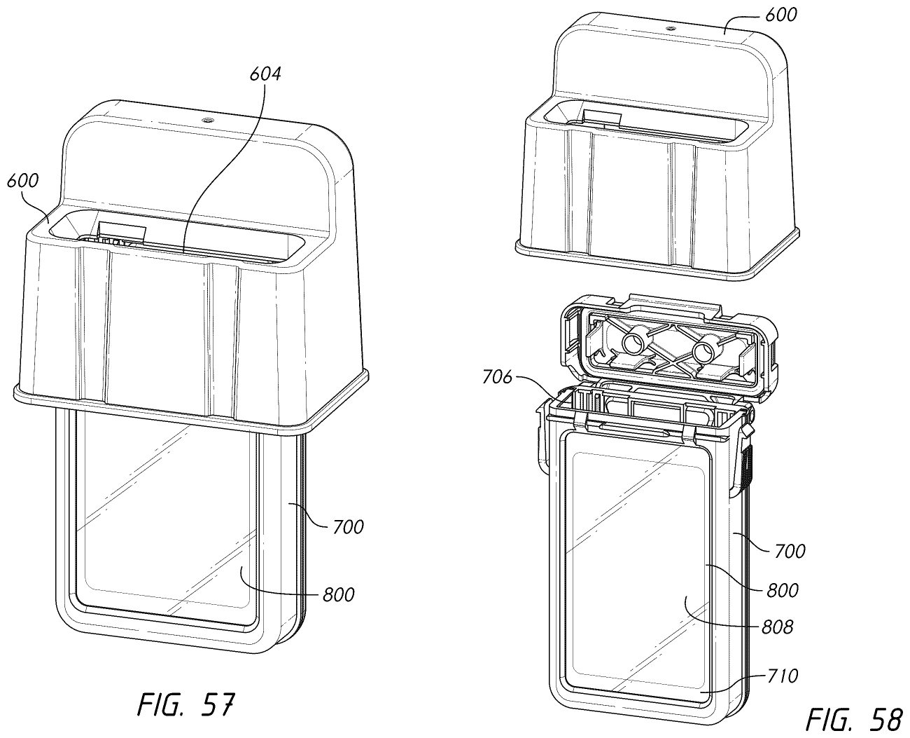

FIG. 57 is a method of use of the transfer funnel 600, the sterile cover 700, and the surgical navigation device 800. FIG. 58 is a method of use of the transfer funnel 600, the sterile cover 700, and the surgical navigation device 800.

The surgical navigation device 800 can be inserted into the sterile cover 700. The surgical navigation device 800 can be inserted through the lumen 604 of the transfer funnel 600. The surgical navigation device 800 can be inserted through the opening 706 of the sterile cover 700. The surgical navigation device 800 can be positioned within the housing 704. The surgical navigation device 800 can be positioned against the transparent layer 710. The display 808 can be visible through the transparent layer 710. The display 808 of the surgical navigation device 800 can be used as a touchscreen through the transparent layer 710 of the sterile housing 700. The transparent layer 710 can overlie the display 808. The transfer funnel 600 can be removed after the surgical navigation device 800 is inserted into the sterile cover 700. The transfer funnel 600 can be single use.

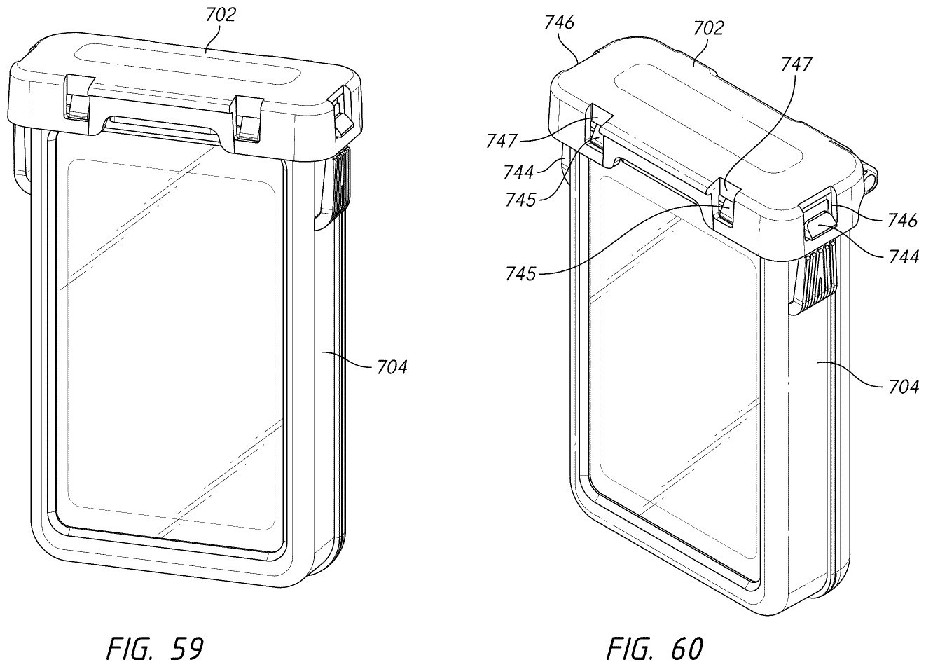

FIG. 59 is a method of use of the sterile cover 700 and the surgical navigation device 800. FIG. 60 is a method of use of the sterile cover 700 and the surgical navigation device 800.

The lid 702 can be closed relative to the housing 704 of the sterile cover 700. The lid 702 can be pivoted to close. The lid 702 can be closed after the transfer funnel 600 is removed. The lid 702 can be closed after the surgical navigation device 800 is inserted into the housing 704. The housing 704 can include one or more projections 744. The lid 702 can include one or more grooves 746. The housing 704 can include one or more additional projections 745. The lid 702 can include one or more additional grooves 747. The lid 702 can pivot relative to the housing 704 to close. The one or more grooves 746 can receive the one or more projections 744. The one or more additional grooves 747 can receive the one or more additional projections 745. The one or more projections 744 and the one or more grooves 746 are configured to be reversable upon the user applying a force to the one or more projections 744. The one or more one or more projections 744 can be pushed inward by the user to disengage the one or more projections 744 and the one or more grooves 746. The one or more additional grooves 747 and the one or more additional projections 745 can provide additional resistance to opening. In some embodiments, the user must use two hands to disengage the lid 702. In some embodiments, the user must use two hands to disengage the projections 744. In some embodiments, the user must use considerable force to disengage the lid 702. The one or more projections 744 and the one or more grooves 746 can be designed with a considerable biasing force to firmly and securely lock. The one or more projections 744 can be biased to a locked configuration. The one or more projections 744 and the one or more grooves 746 can be designed such that intentional and forceful movement is needed to disengage the lid 702. The one or more additional projections 745 and the one or more additional grooves 747 can be designed such that intentional and forceful movement is needed to disengage the lid 702.

As described herein, the sterile cover 700 can include features to provide a liquid resistant seal. The lid 702 and the housing 704 can include a liquid resistant seal. The housing can include a liquid resistant seal between mating components. The one or more liquid resistant seals are designed to maintain sterility between the non-sterile surgical navigation device 800 and the outside environment.

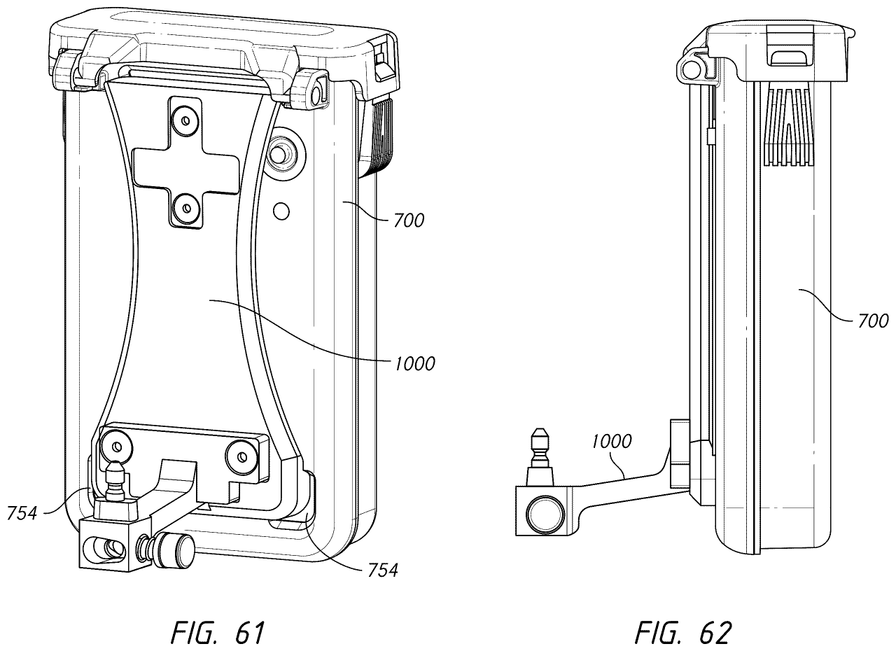

FIG. 61 is a method of use of the sterile cover 700, the surgical navigation device 800, and a bracket 1000. FIG. 62 is a method of use of the sterile cover 700, the surgical navigation device 800, and the bracket 1000. Referring to FIGS. 39 and 41, the housing 704 can include one or more magnets 712. The magnets 712 can be spaced apart along the rear housing 716. The magnets 712 can couple to the bracket 1000. The bracket 1000 can couple to portions of a jig of a joint replacement procedure. The bracket 1000 can include magnets configured to attract the magnets 712 of the housing 700.

The rear housing 716 can include one or more bracket alignment grooves 754. The bracket alignment grooves 754 can receive a portion of the bracket 1000. The bracket alignment grooves 754 can be formed in any surface of the sterile cover 700. The bracket alignment grooves 754 can be formed in the rear housing 716. The bracket alignment grooves 754 can be formed in the sides of the housing 704. The bracket alignment grooves 754 can be formed in the front housing 708. The sterile cover 700 can couple with the jig with multiple points of contact. The bracket alignment grooves 754 can facilitate alignment of magnets of the sterile cover 700 and magnets of the bracket 1000. Once the magnets of the sterile cover 700 couple with the and magnets of the bracket 1000, the interconnection can form a rigid body that inhibits movement of the sterile cover 700 relative to the bracket 1000. The sterile cover 700 can include magnets that couple to corresponding magnets of the bracket 1000. The sterile cover 700 can include a groove that couples to corresponding projection of the bracket 1000. For example, one or more projections of the bracket 1000 can be inserted into one or more grooves or channels along the rear housing 716 of the sterile cover 700. The coupling between the bracket 1000 and the sterile cover 700 can reversible.

The sterile cover 700 can couple to the bracket 1000 using any method described herein. In some embodiments, the sterile cover 700 can have one or more shaped grooves that interlock with the projection of the bracket 1000. The projection of the bracket 1000 can be biased to facilitate attachment of the bracket 1000 and the sterile cover 700. The projection can be biased toward a locking position in which the projection is moved toward the groove. The user can apply a force to the projection of the bracket 1000 or the sterile cover 700 to release the sterile cover 700. In some embodiments, the sterile cover 700 can include a projection that couples to corresponding groove of the bracket 1000. The sterile cover 700 can include a lever-like structure which can pivot in order to lock and unlock with the bracket 1000. Other configurations are contemplated.

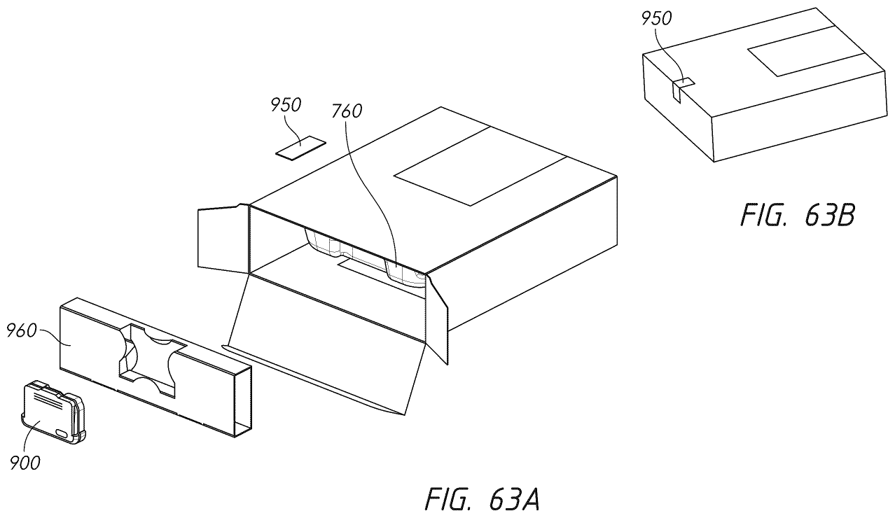

FIGS. 63A and 63B are views of a single use kit. The single use kit can be used for a specific joint procedure. The single use kit can be used for a knee replacement. The single use kit can be used for a hip replacement. The single use kit can include the battery pack 900. The battery pack 900 can be used for a specific joint procedure. The battery pack 900 can be licensed for a knee procedure. The battery pack 900 can be licensed for a hip procedure. The battery pack 900 can be licensed for a single procedure. The single use kit can include the transfer funnel 600 and the sterile cover 700. The single use kit can be shipped with multiple kits. The single use kit can include a tamper seal 950. The single use kit can include a carton insert 960. The carton insert 960 can receive the battery pack 900. The single use kit can include sterile packaging 760. The sterile packaging 760 can include the transfer funnel 600 and the sterile cover 700. The single use kit can be delivered in a closed and assembled state.

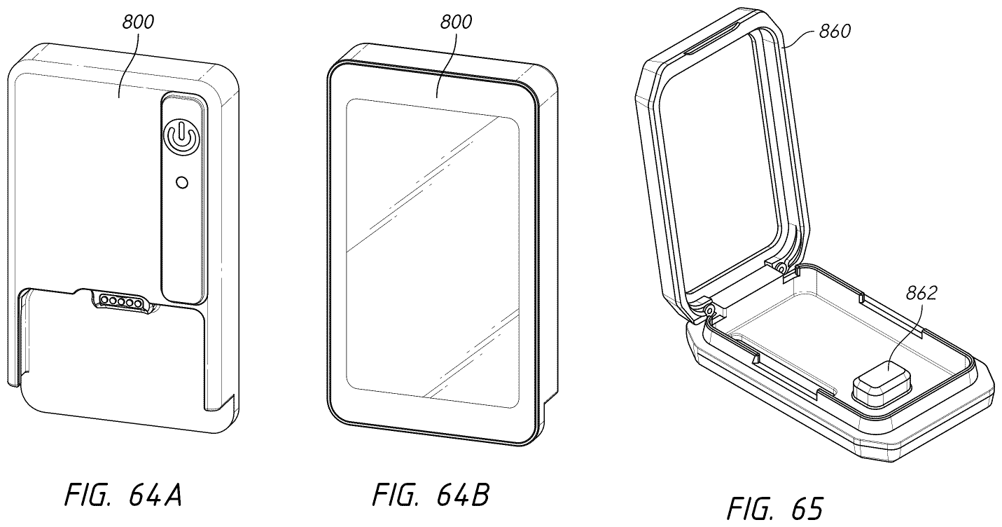

FIGS. 64A and 64B are views of the surgical navigation device 800. The surgical navigation device 800 can be shipped individually. The surgical navigation device 800 can be shipped separately from the single use kit.

FIG. 65 is a view of a storage tray 860. The storage tray 860 can receive the surgical navigation device 800 for storage. The storage tray 860 can include a projection 862 that mates with the surgical navigation device 800. The projection 862 can ensure that the battery pack 900 is removed before storage. The surgical navigation device 800 can be stored between surgical procedures. The surgical navigation device 800 can be stored for reuse.

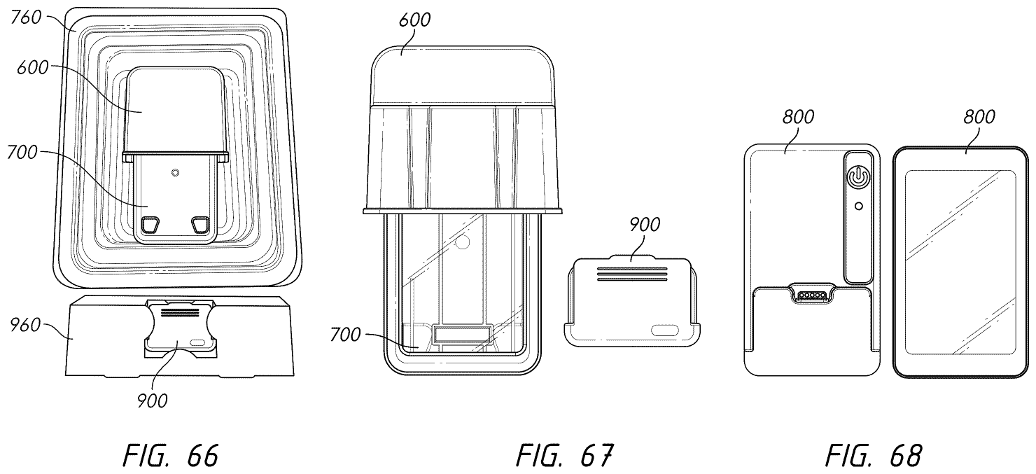

FIG. 66 is a view of a sterile packaging 760 with the transfer funnel 600 and the sterile cover 700. The sterile packaging 760 can be removed from the single use kit. The transfer funnel 600 and the sterile cover 700 can be pre-assembled in the sterile packaging 760. The transfer funnel 600 and the sterile cover 700 can be sterile. The transfer funnel 600 and the sterile cover 700 can arrive pre-assembled for the user. The carton insert 960 can be removed from the single use kit. The carton insert 960 can receive the battery pack 900. The battery pack 900 can be non-sterile.

FIG. 67 is a view of the transfer funnel 600, the sterile cover 700, and the battery pack 900. The transfer funnel 600 and the sterile cover 700 can be removed from the sterile packaging 760. The battery pack 900 can be removed from the carton insert 960. These single use components can be shipped together in the single use kit. These single use components can be configured for use in a single surgical procedure. These single use components can be disposed of after the surgical procedure. The battery pack 900 can be labeled for a specific type of surgery. The battery pack 900 can be labeled for a knee procedure. The battery pack 900 can be licensed for only a knee procedure. The battery pack 900 can be authorized for only a knee procedure.

FIG. 68 are views of the surgical navigation device 800. The surgical navigation device 800 can be shipped separately. The surgical navigation device 800 can be designed for reuse. The hospital or other surgical arena can have the surgical navigation device 800 from a previous surgery. The hospital or other surgical arena can reuse the surgical navigation device 800. The surgical navigation device 800 can be stored without the battery pack 900. The surgical navigation device 800 can be used for any type of surgical procedure. The surgical navigation device 800 for a certain number of procedures.

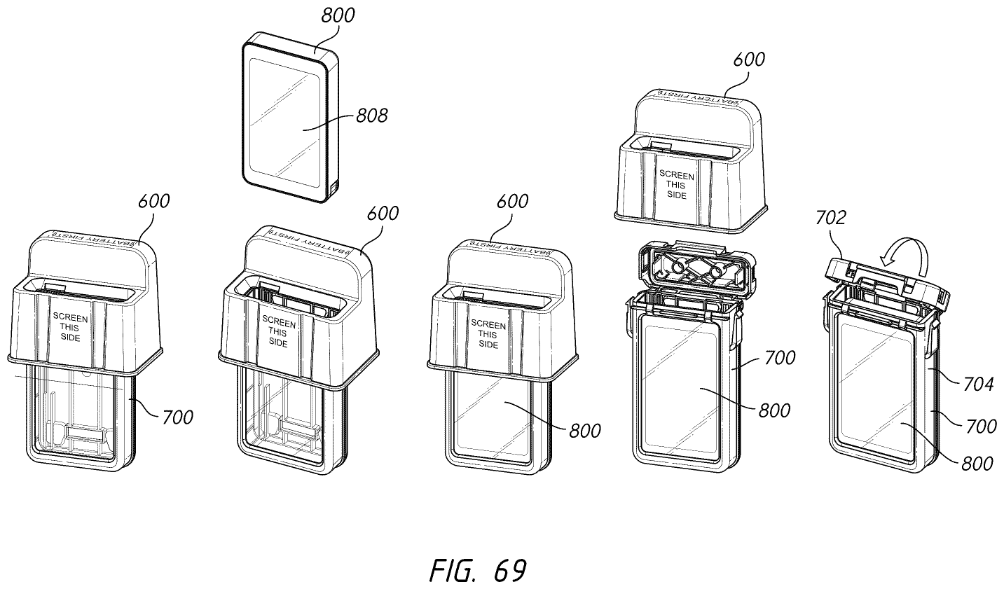

FIG. 69 is a method of use. The transfer funnel 600 and the sterile cover 700 can arrive pre-assembled. The transfer funnel 600 can include instructions for the user regarding aseptic transfer. The transfer funnel 600 can include instructions for orienting the surgical navigation device 800. The transfer funnel 600 can include instructions for orienting the surgical navigation device 800 relative to the transfer funnel. The transfer funnel 600 can include instructions can include instructions on the direction that the display 808 should face. The transfer funnel 600 can include instructions for one or more steps. The transfer funnel 600 can include instructions to install the battery pack 900. The transfer funnel 600 can include instructions to install the battery pack 900 with the surgical navigation device 800 before inserting the surgical navigation device 800. The surgical navigation device 800 can be positioned relative to the transfer funnel 600 and the sterile cover 700. The surgical navigation device 800 can be inserted into the transfer funnel 600 and the sterile cover 700. The transfer funnel 600 can be removed from the sterile cover 700. The lid 702 of the sterile cover 700 can be closed. The surgical navigation device 800 can be contained within the sterile cover 700. The sterile cover 700 can provide a sterile barrier between the surgical navigation device 800 and the surgical field.

Although these inventions have been disclosed in the context of certain preferred embodiments and examples, it will be understood by those skilled in the art that this application extends beyond the specifically disclosed embodiments to other alternative embodiments and/or uses of the invention and obvious modifications and equivalents thereof. In addition, while a number of variations of the inventions have been shown and described in detail, other modifications, which are within the scope of the inventions, will be readily apparent to those of skill in the art based upon this disclosure. It is also contemplated that various combinations or sub-combinations of the specific features and aspects of the embodiments may be made and still fall within the scope of the application. Accordingly, it should be understood that various features and aspects of the disclosed embodiments can be combined with or substituted for one another in order to form varying modes of the disclosed embodiments. Thus, it is intended that the scope of the present invention herein disclosed should not be limited by the particular disclosed embodiments described above but should be determined only by a fair reading of the claims that follow.

Similarly, this method of disclosure, is not to be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

1. A system for a surgical procedure comprising:
a sterile cover comprising a housing and a lid; and
a surgical navigation device comprising at least one inertial sensor and a display,
wherein the surgical navigation device is configured to be inserted into the housing of the sterile cover, wherein the lid is configured to close to provide a sterile barrier separating the surgical navigation device from a sterile field.

2. The system of claim 1, further comprising a transfer funnel.

3. (canceled)

4. The system of claim 2, wherein the transfer funnel comprises an alignment slot configured to prevent inserting the surgical navigation device in an incorrect orientation relative to the sterile cover.

5. The system of claim 2, wherein the transfer funnel comprises instructions to the user related to the orientation of the surgical navigation device.

6. (canceled)

7. The system of claim 1, wherein the housing comprises one or more magnets for coupling to a bracket of a jig.

8. The system of claim 1, wherein the housing comprises a seal between the lid and the housing of the sterile cover.

9. The system of claim 1, wherein the housing comprises a front housing, a rear housing, and a seal between the front housing and the rear housing.

10. (canceled)

11. The system of claim 1, further comprising a battery pack.

12. (canceled)

13. (canceled)

14. (canceled)

15. The system of claim 1, wherein the lid is configured to pivot approximately 90 degrees relative to the housing.

16. (canceled)

17. (canceled)

18. The system of claim 1, wherein the housing comprises projections along three sides of the housing configured to engage grooves along three sides of the lid.

19. (canceled)

20. (canceled)

21. (canceled)

22. (canceled)

23. (canceled)

24. (canceled)

25. (canceled)

26. (canceled)

27. (canceled)

28. A method for a surgical procedure comprising:
providing a sterile cover comprising a housing and a lid;
providing a surgical navigation device comprising at least one inertial sensor and a display;
inserting the surgical navigation device into the housing of the sterile cover; and
closing the lid.

29. (canceled)

30. (canceled)

31. (canceled)

32. (canceled)

33. (canceled)

34. The method of claim 28, further comprising disposing the sterile cover after the surgical procedure and cleaning the surgical navigation device for reuse after the surgical procedure.

35. The method of claim 28, wherein inserting the surgical navigation device into the housing of the sterile cover comprises inserting the surgical navigation device in a single orientation.

36. The method of claim 28, further comprising coupling a battery pack to the surgical navigation device.

37. The method of claim 36, wherein the battery pack comprises data that provides the type of license.

38. The method of claim 36, wherein the battery pack and the surgical navigation device compute a keyed-hash message authentication code to verify the authenticity of the battery pack.

39. A system for a surgical procedure comprising:
a surgical navigation device comprising at least one inertial sensor and a display; and
a battery pack, wherein the battery pack is configured to be coupled to the surgical navigation device.

40. The system of claim 39, wherein the battery pack comprises data that provides the type of license.

41. The system of claim 39, wherein the battery pack and the surgical navigation device compute a keyed-hash message authentication code to verify the authenticity of the battery pack.

42. The system of claim 39, wherein the battery pack comprises a counter, wherein the battery pack is programmed for a single use.

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)
